(19) United States
(12) Patent Application Publication
(10) Pub. No.: US 2020/0051677 A1
(43) Pub. Date: Feb. 13, 2020

(54) METHODS, SYSTEMS, AND COMPUTER-READABLE MEDIA FOR PATIENT ENGAGEMENT AND CARE COORDINATION

(71) Applicant: GAZUNTITE LLC, Washington, DC (US)

(72) Inventor: John Harrison, Washington, DC (US)

(21) Appl. No.: 16/657,990

(22) Filed: Oct. 18, 2019

Related U.S. Application Data
(63) Continuation-in-part of application No. 15/412,642, filed on Jan. 23, 2017, now abandoned.
(60) Provisional application No. 62/281,306, filed on Jan. 21, 2016.

(51) Int. Cl. G16H 15/00; G16H 10/60; G06Q 30/04; G16H 40/67; G06Q 20/10; G06Q 20/14; G06F 3/0481; G06F 3/0484

(52) U.S. Cl. CPC G16H 15/00; G16H 10/60; G06Q 30/04; G16H 40/67; G16H 40/20; G06Q 20/14; G06F 3/0481; G06F 3/04845; G06Q 20/108

(57) ABSTRACT

Systems, methods, and computer readable-media are provided for patient engagement and care coordination. A first graphical user interface is displayed on a display. The first graphical user interface is associated with a first functionality and is of a first size. An input for the selection of a second functionality associated with a second graphical user interface is received. The second graphical user interface is different from the first graphical user interface. In response to receiving the input, the first graphical user interface is resized to a second size. The second graphical user interface is displayed on the display with the resized first graphical user interface. The second graphical user interface is of the second size.

### CROSS REFERENCE TO RELATED APPLICATIONS

The present application is a continuation-in-part of U.S. patent application Ser. No. 15/412,642, filed Jan. 23, 2017, which claims the benefit of and priority to U.S. Provisional Application Serial No. 62/281,306, filed on Jan. 21, 2016, the entire contents of which are incorporated herein by reference.

### TECHNICAL FIELD

The present disclosure generally relates to methods and systems for providing health care and in particular to a patient monitoring platform.

### BACKGROUND

There is a growing trend of health care providers moving towards outpatient care and away from inpatient care. While outpatient care provides monetary benefit to the patients by reducing costs of seeking health care, they also increase the responsibility that a patient must bear in order for the patient to receive the quality of health care necessary to improve his or her health. As a follow up to many medical procedures, patients are required to undergo various steps of rehabilitation and each step of rehabilitation may include multiple additional steps that the patient must complete. Typically the more serious the medical procedure, the more lengthy the rehabilitation process is for the patient. Therefore, it is likely that patients that are most vulnerable to health failure are also the patients that must bear the most responsibilities in order to receive the quality of health care necessary to improve their lives.

Additionally, hospitals lose money every time a patient returns to the hospital within a short period of time because the hospitals are unable to charge the patient or the patient's insurance provider for the services rendered by the hospital. Thus, lack of tools that provide seamless coordination between a patient and care provider leads to inefficient rendering of health care services by the care providers and lower quality of care experienced by the patients.

### SUMMARY

According to an aspect of the present disclosure, systems and methods are provided that address the above mentioned needs. In an aspect of the present disclosure, a method of presenting information related to a patient record to a user is provided. The method includes displaying a first graphical user interface on a display, the first graphical user interface associated with a first functionality, wherein the first graphical user interface is of a first size. An input for the selection of a second functionality associated with a second graphical user interface is received, wherein the second graphical user interface is different from the first graphical user interface. In response to receiving the input, the first graphical user interface is resized to a second size and displaying the second graphical user interface on the display with the resized first graphical user interface, wherein the second graphical user interface is of the second size.

In another aspect of the present disclosure, the resized first graphical user interface occupies only one-half of the display.

In another aspect of the present disclosure, the method further includes displaying the second graphical user interface above the resized first graphical user interface.

In still another aspect of the present disclosure, the method further includes displaying the second graphical user interface below the resized first graphical user interface.

In still another aspect of the present disclosure, the size of the second size is based on the size of the display displaying the first graphical user interface.

According to another aspect of the present disclosure, a method of tracking follow up of health tasks for a patient is provided. The method includes receiving a health task associated with a care plan of a first user. A start time for the health task is identified. A determination is made as to whether the start time for the health task has occurred. In response to determining that the start time for the health task has occurred, a determination is made as to whether the health task is completed. In response to determining that the health task is not completed, a determination is made as to whether the end time for the health task has occurred. In response to determining that the end time for the health task has occurred, a secondary alert mechanism associated with the health task is identified, based on the care plan.

In yet another aspect of the present disclosure, the secondary alert mechanism is an interactive voice response and an alert is transmitted to the first user via a telephone call.

In yet another aspect of the present disclosure, the secondary alert mechanism is an interactive email response and an alert is transmitted to the first user via an email.

In yet another aspect of the present disclosure, the secondary alert mechanism is an interactive text response and an alert is transmitted to the first user using a Short Message Service.

In still another aspect of the present disclosure, prior to the step of identifying the secondary alert, a determination is made as whether a threshold amount of time has elapsed since the end time for the health task occurred. In response to determining that the threshold amount of time has not elapsed, a primary alert mechanism associated with the health task is identified, based on the care plan. An alert to the first user via the primary alert mechanism is transmitted.

In still another aspect of the present disclosure, the method further includes determining whether a plurality of secondary alert mechanisms are associated with the health task. In response to determining that the plurality of secondary alert mechanisms are associated with the health task, the highest ranked secondary alert mechanism from amongst the plurality of secondary alert mechanisms is identified, based on the care plan. An alert to the first user via the highest ranked secondary alert mechanism is transmitted.

In still another aspect of the present disclosure, the method further includes determining whether a threshold amount of time has elapsed since the transmission of the alert to the first user. In response to determining that the threshold amount of time has elapsed, a determination is made as to whether the health task is completed. In response to determining that the health task is not completed, the second highest ranked secondary alert mechanism is identifying, based on the care plan. The alert to the first user via the second highest ranked secondary alert mechanism is transmitted.

In still another aspect of the present disclosure, the method further includes in response to determining that the health task is completed, a determination is made as to whether a measured health attribute value associated with the health task exceeds a threshold value. In response to determining that the measured health attribute value exceeds the threshold value, an alert message to a care provider of the first user is transmitted.

In yet another aspect of the present disclosure, prior to the step of receiving the health task, an input to activate the care plan is received. A determination is made as to whether or not the care plan is successfully activated. In response to determining that the care plan is successfully activated, in a calendar application associated with the first user, entries for all health tasks associated with the care plan are automatically created.

According note aspect of the present disclosure, a method of saving a voice call recording is provided. The method includes recording a voice call between a first user and a second user. A determination is made as to whether the voice call has ended. in response to determining that the voice call has ended, a request to save the voice call recording is displayed to the first user and the second user. An input from the first user indicating to save the voice call recording is received. In response to receiving the input from the first user indicating to save the voice call recording, a notification to the second user indicating that the first user approves saving the voice call recording is transmitted. An input from the second user is received. A determination is made as to whether the input from the second user approves saving the voice call recording. In response to determining that the input from the second user approves saving the voice call recording, saving the voice call recording in a data storage unit.

In yet another aspect of the present disclosure, the method further includes determining whether the first user is a care provider. In response to determining that the first user is a care provider and the second user approves saving the voice call recording, a request to the first user for generating a bill to a health insurance provider is displayed. An input from the first user approving the request for generating the bill is received. A message to the health insurance provider is transmitted, wherein the message includes a generated bill for the services rendered by the first user and a copy of the saved voice call recording.

In yet another aspect of the present disclosure, the method further includes determining whether the first user and the second user are care providers. In response to determining that the first user and the second user are care providers and the second user approves saving the voice call recording, a request to the first and the second user for generating a bill to a health insurance provider is displayed. An input from the first user and the second user approving the request for generating the bill is received. A message to the health insurance provider is transmitted, wherein the message includes a generated bill for the services rendered by the first user and a copy of the saved voice call recording. A message to the health insurance provider is transmitted, wherein the message includes a generated bill for the services rendered by the second user and a copy of the saved voice call recording.

In yet another aspect of the present disclosure, the method further includes, in response to determining that the input from the second user does not approve saving the voice call recording, deleting the voice call recording.

In still another aspect of the present disclosure, the method further includes, associating the saved voice call recording with a profile of the first user and a profile of the second user.

### BRIEF DESCRIPTION OF THE DRAWINGS

The above and other aspects, features, and advantages of the present disclosure will become more apparent in light of the following detailed description when taken in conjunction with the accompanying drawings, in which:

FIG. 1 is a schematic of one illustrative embodiment in accordance with the present disclosure of a patient monitoring platform in communication with one or more external systems;

FIG. 2A-2B are graphical user interfaces displaying graphical items that enable users to log into the patient monitoring platform;

FIG. 2C is a graphical user interface displaying graphical items that present information related to a user, such as a patient;

FIG. 3A-FIG. 3H are graphical user interfaces displaying graphical items that present information related to viewing, interacting with, and uploading health records of a user to the patient monitoring platform;

FIG. 4A-FIG. 4Q are graphical user interfaces displaying graphical items that present information related to searching for a care provider and scheduling an appointment with a care provider;

FIG. 5A-FIG. 5B are graphical user interface displaying graphical items that present information related to managing payments using the patient monitoring platform;

FIG. 6A-FIG. 6B are graphical user interfaces displaying graphical items that present multiple graphical user interfaces simultaneously

FIG. 6C-FIG. 6E is a graphical user interface displaying graphical items that enable saving and associating a file to user's profile of the patient monitoring platform;

FIG. 6F-FIG. 6G is a graphical user interface displaying graphical items that present one or more folders associated with a user's profile of the patient monitoring platform;

FIG. 7A-FIG. 7Z11 are graphical user interfaces displaying graphical items that enable activation of a care plan;

FIG. 8A-FIG. 8B are graphical user interfaces displaying graphical items that present information related to a user, such as a care provider;

FIG. 9A-FIG. 9C are graphical user interfaces displaying graphical items that enable billing for telemedicine session;

FIG. 10A-FIG. 10S are graphical user interfaces displaying graphical items that enable creation of a care plan; and

FIG. 11A-FIG. 11I are graphical user interfaces displaying graphical items that enable a care provider to communicate with patients and diagnose alerts from patients.

### DETAILED DESCRIPTION

Healthcare information technology (IT) systems available today are single purpose and lack interoperability. Specifically, providers cannot virtually collaborate, save collaboration, and attach collaboration to an insurance reimbursement claim. Providers also cannot instantly share records with patients and other providers, transfer patients to other providers with records and refer patients to other systems, or provide follow up health plans and monitor and ensure that a patient follow plans. Moreover, among other things, patients cannot collect, save, and share records by multiple means, cannot follow health plans through tasks and alerts, cannot create health plans in a single application, cannot collaborate virtually with providers and have those collaborations saved to their folders, cannot electronically submit out-of-network claims through a clearinghouse, cannot collect, monitor, and understand their health bills, and cannot manage a dependent's health in the same app as their own health management.

Currently, healthcare IT applications are provider-focused, and even then only have a fraction of the features required to efficiently run a physical office. These healthcare IT applications and systems lack the ability to provide virtual/remote services for a fee. Moreover, all lack a patient application for record creation, saving, gathering, sharing across multiple providers, collaboration tools, health bills management and automatic and remote assessment and plan monitoring. Other healthcare IT solutions also lack a number of required features and functions to extend a doctor's office to offer remote care management. Additionally, the functions and features existing healthcare IT solutions do have are not fully interoperable with one another.

The present disclosure is directed to systems and methods of implementing a patient monitoring platform that allows care providers to effectively provide inpatient and outpatient care to their patients and empowers patients to efficiently manage their health records, research, medical care related administrative tasks, and/or interaction with the care providers. The patient monitoring platform described herein alleviates the issues and challenges described above.

As used herein, the term “care provider” refers to a doctor, physician's assistant, a nurse, or any other care provider, including support personnel, or the like, providing medical care and/or performing a medical procedure. As used herein, the term “vitals data” refers to any information related to health of a patient, including but not limited to, heart rate, body weight, body mass index, etc. Throughout this description, the term “physical attribute” refers to any information related to physical information of a patient, including but not limited to, patient's height, skin color, hair color, eye color, etc. Throughout this description, the term “fitness data” refers to any information related to physical activity or inactivity of a patient, including but not limited to, the amount of time spent exercising, the number of steps taken within a certain time period, the number of miles walked within a certain time period, the number of miles ran within a certain time period, etc. Throughout this description, the term “publish” refers to transmitting data to another computing device or making data available for consumption or retrieval by other computing devices. Throughout this description, the term “medical facility” refers to any entity that provides healthcare related services, such as a hospital, a clinic, etc. Throughout this description, the term “call” refers to voice calls through a telephone network or using Voice over Internet Protocol (VoIP) and communications using videotelephony technologies. Throughout this description, the term “user” refers to a person registered with the patient monitoring platform described herein, including, but not limited to, persons who are patients and/or care providers. Throughout this description, the term “graphical items” includes, but is not limited to, images, graphical labels, data entry mechanisms, such as text boxes, radio buttons, drop down lists, drop down menus, any other graphical control elements, etc., or combinations thereof.

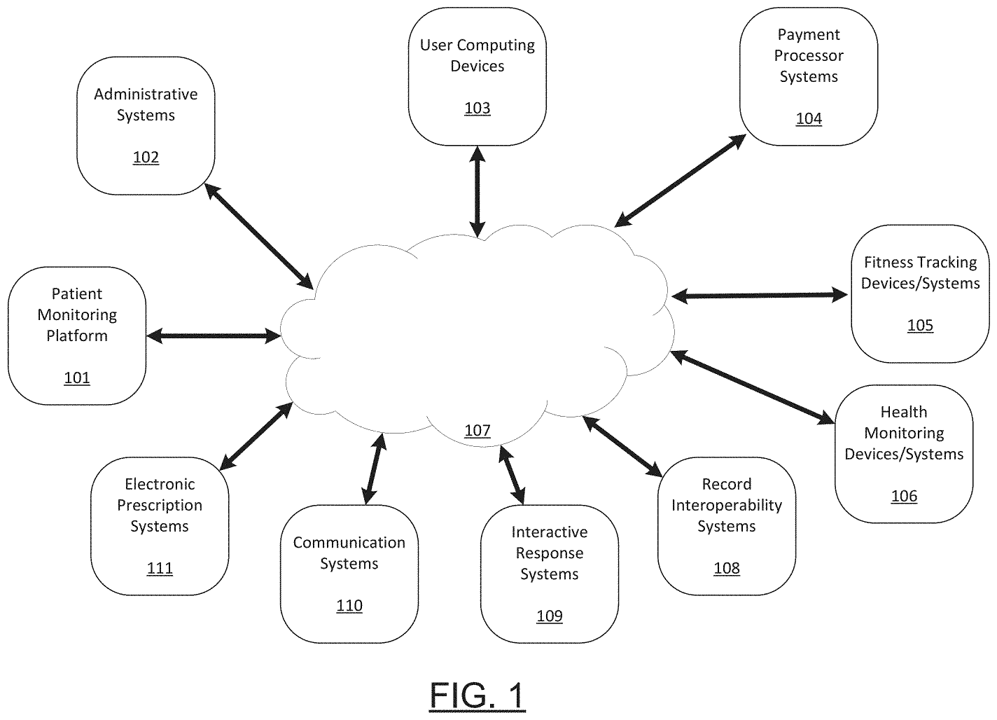

The systems and methods, according to the present disclosure, may be implemented as illustrated in FIG. 1. According to an embodiment, a patient monitoring platform 101 is communicatively coupled to one or more external computing devices and systems via a telecommunications network 107 is shown in FIG. 1. Examples of telecommunications network 107 include, but are not limited to, computer networks, the Internet network, etc. Each of the one or more computing devices and systems shown in FIG. 1 communicate with one another by transmitting and receiving packets via the telecommunications network 107. Examples of the one or more external computing devices and systems shown in FIG. 1 include, but are not limited to, administrative systems 102, user computing devices 103, payment processor systems 104, fitness tracking devices and systems 105, health monitoring devices and systems 106, record interoperability systems 108, interactive response systems 109, communication systems 110, and electronic prescription systems 111.

The administrative systems 102 are systems that are configured to assist medical facilities, such as hospitals, clinics, etc., manage administrative tasks, such as scheduling of medical procedures for patients of care providers associated with the medical facility, maintaining electronic medical records (EMR) of the patients, including creating or updating of the EMR, and the like. In some embodiments, the patient monitoring platform 101 is configured to synchronize schedules of the care providers registered with patient monitoring platform 101 using schedule-related data from respective administrative systems 102 of the care providers. In some embodiments, the patient monitoring platform 101 transmits the schedule-related data of a care provider to the administrative system 102 associated with the care provider in order to update the schedule-related data on the administrative system 102. In some embodiments, the patient monitoring platform 101 initiates the process of scheduling a medical procedure for a patient of a care provider by transmitting to the administrative system 102 of the care provider a request to schedule the medical procedure for the patient. In some embodiments, the patient monitoring platform 101 accesses health portals to retrieve the EMR of a patient in response to receiving a request to upload the EMR of the patient to the health monitoring platform 101.

The user computing devices 103 are devices and systems used by users of the patient monitoring platform 101 to log into and access the patient monitoring platform 101. Examples of the user computing devices 103 include, but are not limited to, desktop computers, laptop computers, tablet computers, smartphones, other mobile computing devices, etc. A user interacts with the patient monitoring platform 101 by installing an application of the patient monitoring platform 101 on to a user computing device 103 of the user. For example a user may download an application of the patient monitoring platform 101 on a mobile computing device, and using the user interfaces provided by the application, the user interacts with the functionalities of the patient monitoring platform 101. The payments processor systems 103 are devices and systems configured to exchange a value, such as money, credit, and the like, from one user of the system to another user of the system. The patient monitoring platform 101 is configured with one or communication protocols necessary to provide transaction-related data or transaction messages to the payment processor systems. Examples of the payment processor systems 103 include, but are not limited to, Stripe, PayPal, ApplePay, etc.

The fitness devices and/or systems 105 are devices and systems configured to observe and capture data related to a user's physical activity. Based the captured physical activity related data, the fitness devices and/or systems 105 are configured to calculate metrics related to the user's fitness and, based on the calculated metrics, generate fitness data and store the fitness data. Examples of the fitness devices and/or systems 105 include, but are not limited to, step counters, smart watches, smart bands, etc. Examples of the metrics calculated by the fitness devices and/or systems 105 include but are not limited to, distance walked, distance run, heartbeat, etc. In some embodiments, users registered with the patient monitoring platform 101 associate their fitness devices and/or systems 105 with their profile on the patient monitoring platform 101, and the patient monitoring platform 101 automatically retrieves fitness data of a user from the user's fitness devices and/or systems 105 associated with the patient monitoring platform 101.

The health monitoring devices and/or systems 106 are devices and systems configured to observe and capture data related to the user's health including one or more of the user's vitals. The health monitoring devices and/or systems 106 generate and store health data of the user based on metrics calculated from health-related data captured by one or more sensors of the health monitoring devices and/or systems 106. Examples of the health monitoring devices and/or systems 106 include, but are not limited to, heart rate monitors, pulse rate monitors, etc. As with the fitness devices and/or systems 105, the patient monitoring platform 101 is configured to automatically retrieve data related to the health of the user from the user's health monitoring devices and/or systems 106 associated with the user's profile on the patient monitoring platform 101.

The record interoperability systems 108 are systems and/or devices configured to provide access to health records of a patient, including electronic medical record (EMR) of the patient. The record interoperability systems 108 are configured to allow users, including external systems, to search for health records, interact with health records, view health records, exchange health records by downloading health records from the record interoperability systems 108 and uploading health records to the record interoperability systems 108. The patient monitoring platform 101 is communicatively coupled with the record interoperability systems 108 in order to allow the users of the patient monitoring platform 101 to access, view, download, and/or upload the health records from and to the record interoperability systems 108. In some embodiments, the patient monitoring platform 101 is configured to transmit and receive data, including health records, using one or more of application programming interfaces (APIs) of the record interoperability systems 108.

In some embodiments, the patient monitoring platform 101 is communicatively coupled to the interactive response systems 109 via network 107, as shown in FIG. 1. The interactive response systems 109 are systems and/or devices configured to communicate with a person via voice, text, and/or email. The interactive response systems 109 are also configured to receive inputs from the persons with whom the interactive response systems 109 are communicating. The inputs from the persons that the interactive response systems 109 are communicating with are inputs in response to the one or more prompts, questions, and/or requests presented by the interactive response systems 109. In some embodiments, the patient monitoring platform 101 is configured to transmit requests to the interactive response systems 109 to initiate communication with a user of the patient monitoring platform 101. For example, in response to not receiving an appropriate input from a user at a particular time, the patient monitoring platform 101 transmits a message requesting a voice communication with the user, the telephone number of the user, and/or one or more prompts and/or questions or a series of prompts and/or questions to the interactive operating systems 109 for a voice communication with the user. The interactive response systems 109 conduct the voice communication with the user by placing a telephone call with the user and present to the users, via voice, the prompts and/or questions received from the patient monitoring platform 101. The interactive response systems 109 transmit to the patient monitoring platform 101, the inputs received from the users in response to the prompts and/or questions presented by the interactive response systems 109. The patient monitoring platform 101 stores the user inputs received from the interactive response systems 109 and associates the inputs with a profile of the user associated with the patient monitoring platform 101. Additional details of user profile are described below. Similarly, the patient monitoring platform 101 is configured to transmit a request for a text communication, such as short messaging system (SMS), and/or email communication, and the patient monitoring platform 101 transmits a number where the user can receive a text message and an email address associated with the user, respectively. The patient monitoring platform 101 is configured to transmit secondary alerts of a care plan associated with a user to the user via the interactive response systems 109. Additional details of the secondary alerts of a care plan are described in the context of figures described below.

The communication systems 110 are systems and/or devices configured to receive and accommodate requests to communicate with persons and/or systems. The patient monitoring platform 101 is configured to transmit requests to communicate with users of the patient monitoring platform 101 to the communication systems 110, in response to receiving inputs from a user requesting to communicate with another user of the patient monitoring platform 101. Based on the type of communication requested by the user, the patient monitoring platform 101 transmits a corresponding communication identifier of the requesting user and the user with whom the requesting user desires to communicate. As used herein, the term “communication identifier” includes a number, such as a phone number or a telephone number, or a user identifier, at which a person may receive a voice call or a text message. For example, if an input from a first user to the patient monitoring platform 101 indicates that the first user wishes to communicate via voice or telephone call with a second user of the patient monitoring platform 101, the patient monitoring platform 101 transmits a request to the communication systems 110 to place a voice or telephone call with the second user from the first user and the phone numbers of the first user and the second user. The communication systems 110 connects the first user to the second user via a telephone call and/or VoIP call, and the first user and the second user communicate with each other via the patient monitoring platform 101 using the same user computing device with which they are accessing the patient monitoring platform 101.

The electronic prescription systems 111 are systems and/or devices configured to allow for transmission and/or authorization of prescriptions to one or more pharmacies associated with a user of the patient monitoring platform 101. The patient monitoring platform 101, in response to receiving inputs to transmit a prescription created using the patient monitoring platform 101, transmits a request to the electronic prescription systems 111 in order to submit the prescription to a pharmacy. The pharmacy may be associated with one of the users, for example, a patient for whom the prescription is created, or a care provider that creates the prescription. Additional details of creating, transmitting, and/or accessing or viewing prescriptions using the patient monitoring platform 101 are described in the context of figures described below.

The patient monitoring platform 101 is an application hosted on an application server computer (not shown separately in FIG. 1) that includes one or more processors (not shown separately in FIG. 1) operably coupled to one or more of a memory (not shown separately in FIG. 1). The memory stores instructions to be executed by the one or more processors, and the techniques described herein are performed by the patient monitoring platform 101 in response to the one or more processors executing the instructions stored in the memory. The memory may be any type of hardware device used to store data. The memory may be volatile memory, such as random access memory (RAM) (e.g., dynamic random access memory (DRAM), static random access memory (SRAM), etc.). The memory may be non-volatile memory, such as read-only memory (ROM) (e.g., programmable read-only memory (EPROM), electrically erasable programmable read-only memory (EEPROM), non-volatile RAM (NVRAM), etc.). The memory may also be magnetic, optical, or electrical media. The one or more processors may be any type of suitable processor that is adapted to perform or execute the techniques or operations or instructions described herein. For example, the processors may be hardware processors programmed to perform the techniques described herein pursuant to the instructions in firmware, memory, or other storage, or a combination thereof. Similarly, the processors may also be one or more application specific integrated circuits (ASICs), field programmable gate arrays (FPGAs) that are persistently programmed to perform the techniques or operations described herein. The processors may also be a central processing unit (CPU), a digital signal processor (DSP), a microprocessor, or any other device that incorporates hard wired logic or program logic or both to perform the operations or techniques described herein

The application server computer is configured to execute procedures, such as programs, routines, scripts, or other computer executable commands necessary for supporting the patient monitoring platform 101 and the techniques described herein. In some embodiments, for load balancing purposes, the application server computer comprises of multiple instances of the patient monitoring platform 101 and performs load balancing of the users of the patient monitoring platform 101 using the multiple instances of the patient monitoring platform 101. In some embodiments, the patient monitoring platform 101 includes or is coupled to a HyperText Transfer Protocol (HTTP) server and is configured to serve HyperText Markup Language (HTML) documents that browser programs at computing devices, such as the user computing devices 105, can receive, render, and display.

Each user of the patient monitoring platform 101 is provided login credentials, such as, a username and a password, and once logged in, the user may interact with the graphical user interface (GUI) (not shown separately in FIG. 1) of the patient monitoring platform 101 to utilize the patient monitoring platform 101. As used herein, the term “user” includes any person registered with the patient monitoring platform 101 and provided with login credentials including, but not limited to, a care provider, a patient, or any other person authorized to use the patient monitoring platform 101. For example, while a person who is a patient and who registered with the patient monitoring platform 101 may access, interact with, and/or use the patient monitoring platform 101, the patient may authorize another person (a non-patient user), such as a family member, a trusted friend, etc. to access, interact with, and/or use the patient monitoring platform 101. Similarly, a person who is a care provider and who is registered with the patient monitoring platform 101 may access, interact with, and/or use the patient monitoring platform 101, the care provider may authorize another person (a non-care provider user), such as a nurse, a physician's assistant, etc., to access, interact with, and/or use the patient monitoring platform 101. As described herein, all interactions with the patient monitoring platform 101 performed by a patient or a care provider may also be performed by non-patient user or a non-care provider user, respectively.

The patient monitoring platform 101 generates a profile for each user of the patient monitoring platform 101 and associates activity of the user performed using the patient monitoring platform 101 with the profile of the user. As used herein, a profile of a user is a unique identifier of the user. For example, the patient monitoring platform 101 associates every file saved by the user with the profile of the user and if the patient monitoring platform 101 is requested to retrieve a particular file saved by the user, the patient monitoring platform 101 may use the user's profile information to retrieve the saved file. Each user of the patient monitoring 101 is provided certain access privileges. Every functionality of the patient monitoring platform 101 is associated with a certain access privilege and access to various functionalities of the patient monitoring platform 101 is based upon the access privileges of the user and the access privilege associated with the functionality. For example, a user may access a particular functionality if the access privileges of the user include the access privilege associated with the functionality. In some embodiments, the patient monitoring platform 101 does not display to a user, functionalities whose associated access privilege is not included within the access privileges of the user.

The patient monitoring platform 101 presents multiple user interfaces including, but not limited to, a graphical user interface (GUI) (not shown separately in FIG. 1) that allow a user to interact with and access the functionalities of the patient monitoring platform 101. The GUI of the patient monitoring platform 101 includes one or more graphical items (not shown separately in FIG. 1) which the user interacts with and provides data and/or instructions to the patient monitoring platform 101. Graphical items in a GUI presented by the patient monitoring platform 101 are associated with one or more functionalities of the patient monitoring platform 101. As used herein, the term “graphical items” includes, but is not limited to, images, graphical labels, data entry mechanisms, such as text boxes, radio buttons, drop down lists, drop down menus, any other graphical control elements, etc., or combinations thereof. Throughout this description, the term “navigational graphical items” refers to graphical items that are common to multiple graphical user interfaces of the patient monitoring platform 101 and are configured receive input to cause the patient monitoring platform 101 to display certain application screens and/or GUIs of the patient monitoring platform 101 in response to the selection of the navigational graphical items. Examples of navigational graphical items include, but are not limited to, menus, menu bar, etc., or combinations thereof.

In some embodiments, the GUI presented to a user and the graphical items within the GUI that are presented to the user is based on the access privileges of the user and the access privilege associated with the functionality of the graphical items. For example, the access privilege of a patient will be different and more limited than the access privilege of a care provider, such as a physician, and at least some of the graphical items presented to the care provider will not be presented to the patient due to the access privileges of the physician. The patient monitoring platform 101 presents one more data items to a user or causes one or more data items to be displayed to a user based on the inputs received by the patient monitoring platform 101. Additional details of the GUI, the graphical items, and the data items displayed or presented to the user are provided herein in the context of figures described below.

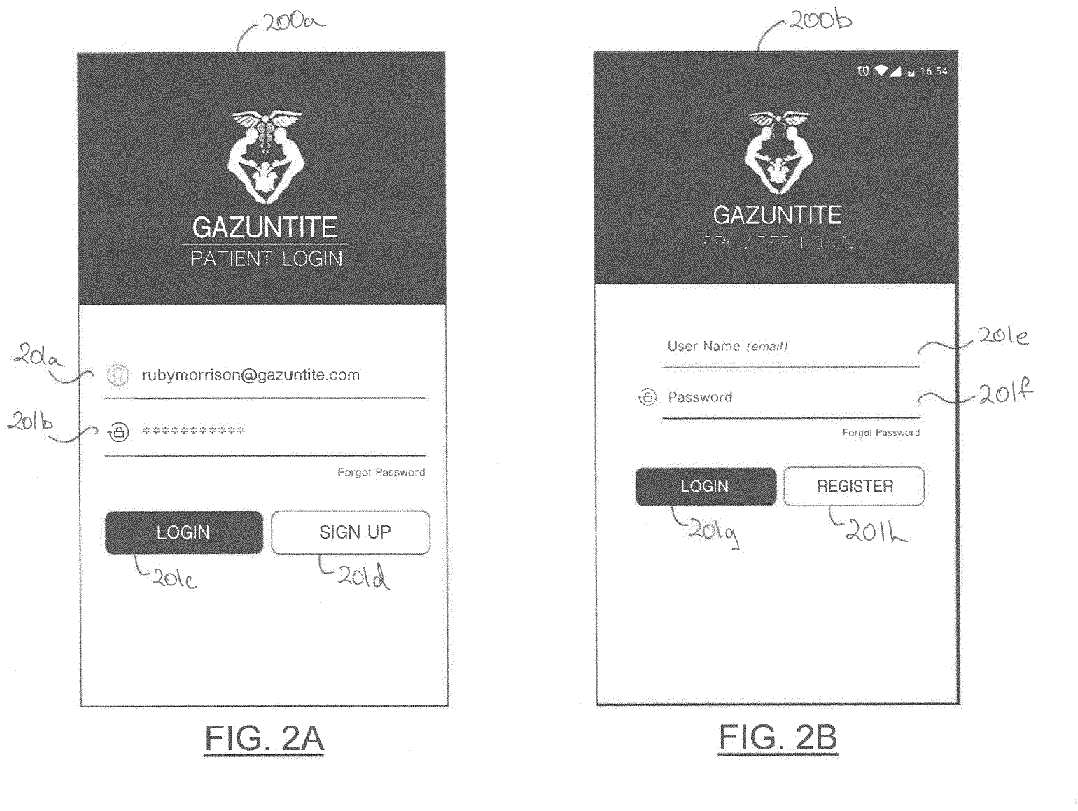

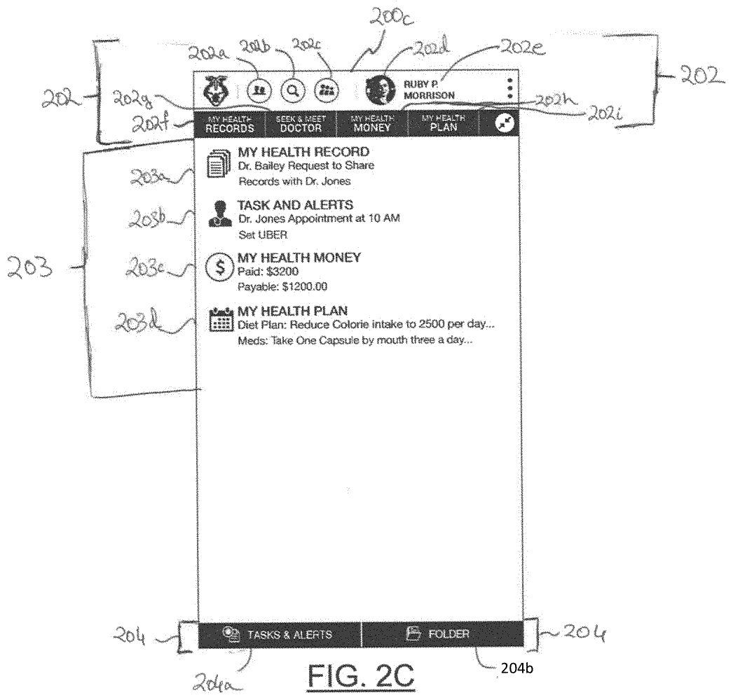

Turning now to FIG. 2A, there is shown a graphical user interface (GUI) 200a of the patient monitoring platform 101 displaying a login screen to a user, such as a patient. The GUI 200a includes graphical items 201a, 201b, 201c, 201d. The graphical items 201a and 201b are configured to receive a user name of the patient and a password associated with the user name. The graphical item 201c is a graphical button configured to receive an input from the patient. An example of the input the graphical item 201c is configured to receive is a touch input that selects the graphical item 201c. Upon selection of the graphical item 201c, the patient monitoring platform 101 determines whether the username and password are accurate and associated with a patient registered with the patient monitoring platform 101. For example, the patient monitoring platform 101 determines whether the username exists within a database of usernames operably coupled to the patient monitoring platform 101 and if the username exists, the patient monitoring platform 101 determines whether the password entered is associated with the username. The graphical item 201d is a graphical button configured to receive an input from a user, and in response to receiving the input, the patient monitoring platform 101 displays graphical items (not shown) configured to receive patient information including biographical, physical, geographical information of the patient, an image of the patient, and/or a username and password to log into the patient monitoring platform 101 in order to sign up or register a new user including, but not limited to, a patient with the patient monitoring platform 101.

In response to a successful verification, the patient monitoring platform 101 displays a different application screen presenting a GUI based on the user logging into the patient monitoring platform 101, such as GUI 200c, shown in FIG. 2C. The GUI 200c is an example user interface for a patient registered with the patient monitoring platform 101 and includes those graphical items that are associated with functionalities whose access privileges are granted to the patient that successfully logged into the patient monitoring platform 101. For example, in the GUI 200c, the patient monitoring platform 101 displays graphical items based on the access privileges of the patient Ruby P. Morrison.

The GUI 200c includes navigational graphical items 202a, 202b, 202c, 202d, 202e, 202f, 202g, 202h, 202i, collectively navigational graphical items 202, and navigational graphical items 204a, 204b, collectively navigational graphical items 204. In response to the selection of a navigational graphical item, the patient monitoring platform 101 displays an application screen associated with selected navigational graphical item. For example, in response to selecting navigational graphical item 202c, the patient monitoring platform 101 presents an application screen displaying those persons who are part of the user's network (not shown separately in FIG. 2C). The patient monitoring platform 101 may display the people within the user's network in multiple ways, including, but not limited to, lists, tabular form, etc. In some embodiments, the patient monitoring platform 101 may also provide suggestions of persons that may be within a certain degree of familiarity with the patient, such as, a friend or relative of the user. In some embodiments, the patient monitoring platform 101 may utilize patient's online activity data, external and/or internal to the patient monitoring platform 101, in providing the suggestions of persons to the user.

In response to the selection of the navigational graphical item 202b, the patient monitoring platform 101 presents an application screen displaying a GUI for interacting with the search functionality of the patient monitoring platform 101. In some embodiments, the patient monitoring platform 101 allows a user to search for various features and/or functionalities of the patient monitoring platform 101 and/or the world wide web using the search functionality of the patient monitoring platform 101. In some embodiments, the patient monitoring platform 101 provides options to select one or more search engines to utilize while the patient monitoring platform 101 searches the user's search terms. In some embodiments, the patient monitoring platform 101 presents the home or search web page of a search engine in response to the selection of the navigational graphical item 202b, and allows the user to interact directly with the web page of the search engine.

In response to the selection of the navigational graphical item 202a, the patient monitoring platform 101 displays one or more users that are registered with the patient monitoring platform 101 and have been associated with the user's profile as contacts of the user. The patient monitoring platform 101 includes a social network that includes all users, patients and care providers, that are registered with the patient monitoring platform 101 and provides users with privacy control options to allow a user to control whether or not the user desires to be shown as part of the network of users registered with the patient monitoring platform 101. The patient monitoring platform 101 may display the people within the network of users in multiple ways, including, but not limited to, lists, tabular form. In some embodiments, the users displayed to the patient are ordered based on the patient's degree of familiarity with the user. In some embodiments, the patient monitoring platform 101 may display an indicator, adjacent to the information displayed of a user, that indicates the patient's degree of familiarity with the user. In some embodiments, the patient monitoring platform 101 displays the registered users within a GUI that includes graphical items that are configured to receive input to add a particular registered user to the patient's network, and, in response to receiving such input, the patient monitoring platform 101 adds the user to the patient's network and stores the association between the user and the patient's network within a data storage unit.

The navigational graphical item 202d is configured to display an image, such as an image of the patient. In response to the selection of the navigational graphical item 202d, the patient monitoring platform 101 presents an application screen displaying a GUI that allows a user to enter or update information related to the user, referred to herein as user profile information. The user profile information includes, but is not limited to, biographical, physical, geographical, etc., information of the user. For example, if the patient Ruby P. Morrison selects the navigational graphical item 202d, then the patient Ruby P. Morrison may update her biographical information, such as date of birth, physical information, such as height and weight of the patient, geographical information, such as the patient's residency location, and the like. The navigational graphical item 202e is a graphical item configured to display text and in FIG. 2C displays the name of the user that logged into the patient monitoring platform 101. In some embodiments, the patient monitoring platform 101 is configured to present the application screen displaying the GUI for entering or updating the user profile information in response to the selection of the navigational graphical item 202e. Similarly, in response to the selection of the navigational graphical items 202f the patient monitoring platform 101 is configured to display application screens and GUIs to view and utilize functionalities related to viewing, uploading, and interacting with health records, labs, diagnostic images, health-related files of the patient. In response to the selection of the navigational graphical items 202g, 202h, 202i, the patient monitoring platform 101 is configured to display applications screens and/or GUIs to view and utilize functionalities related to searching for and interacting with care providers, managing payments, viewing and interacting with care plans of the patient, respectively. Additional details of the above described functionalities are provided herein in context of the figures described below.

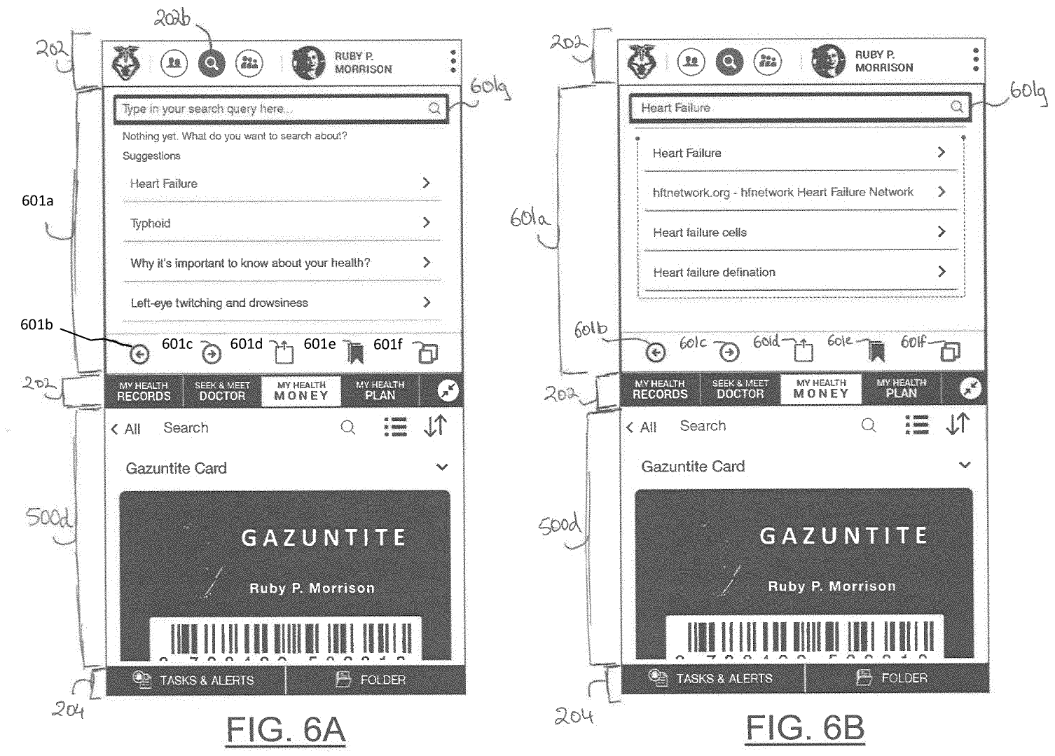

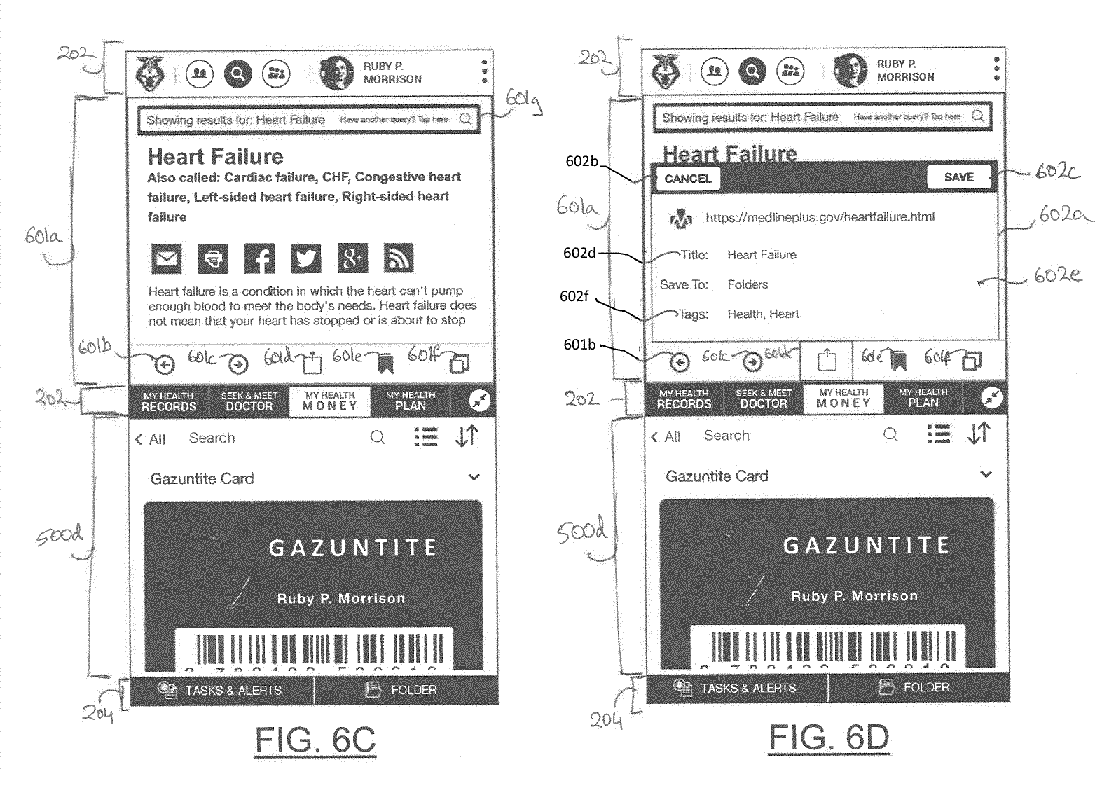

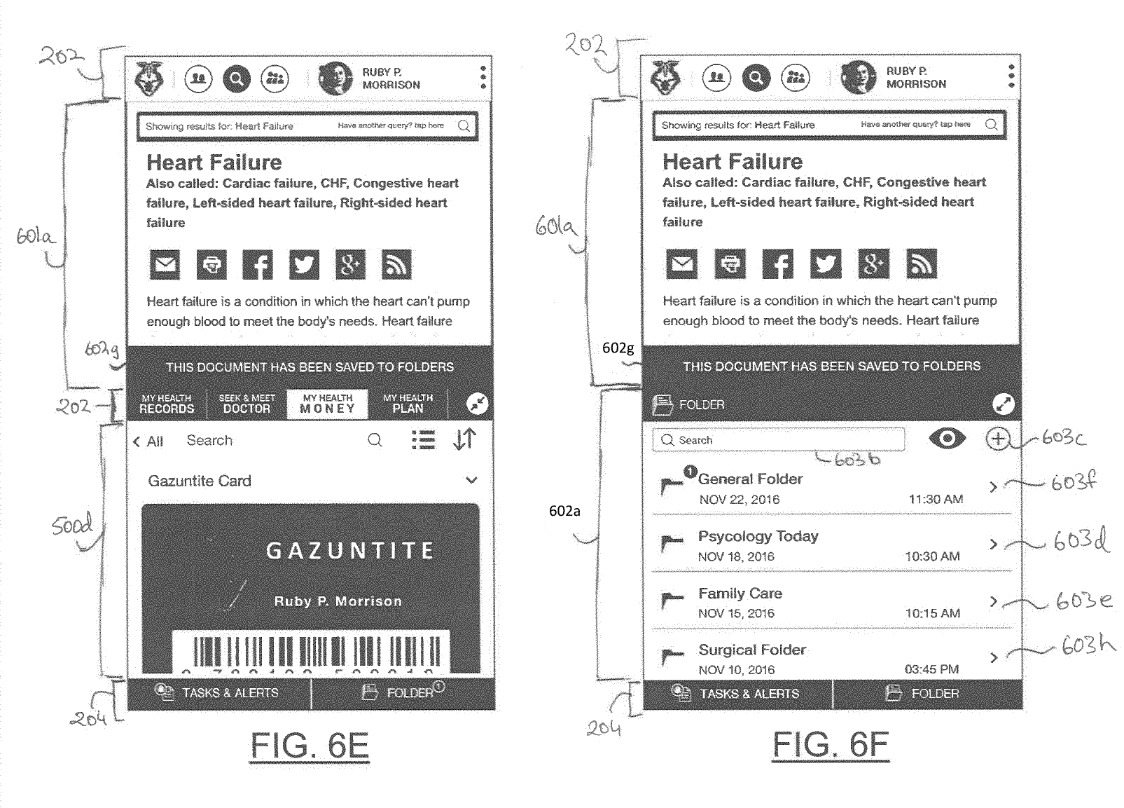

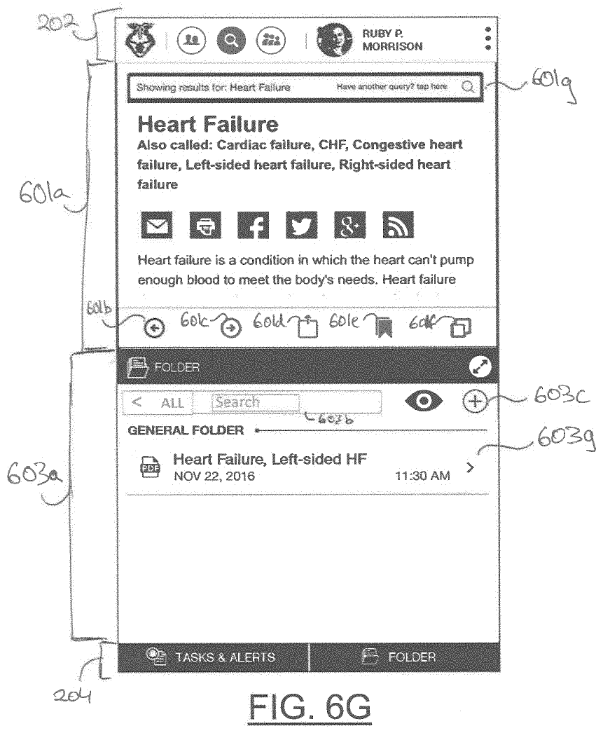

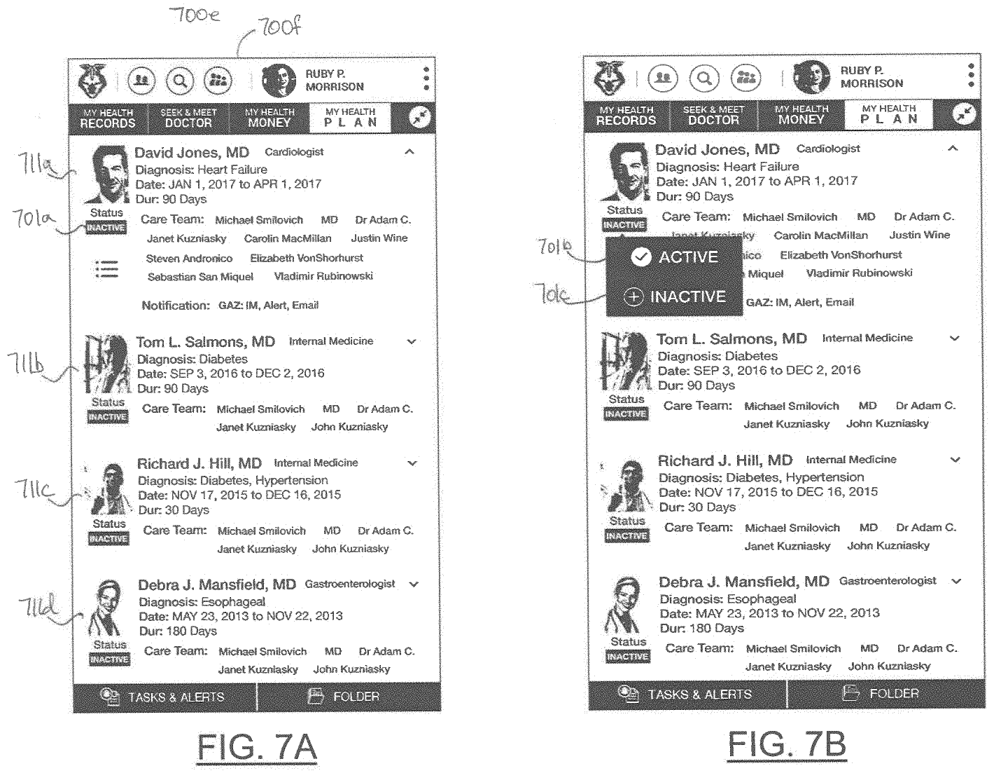

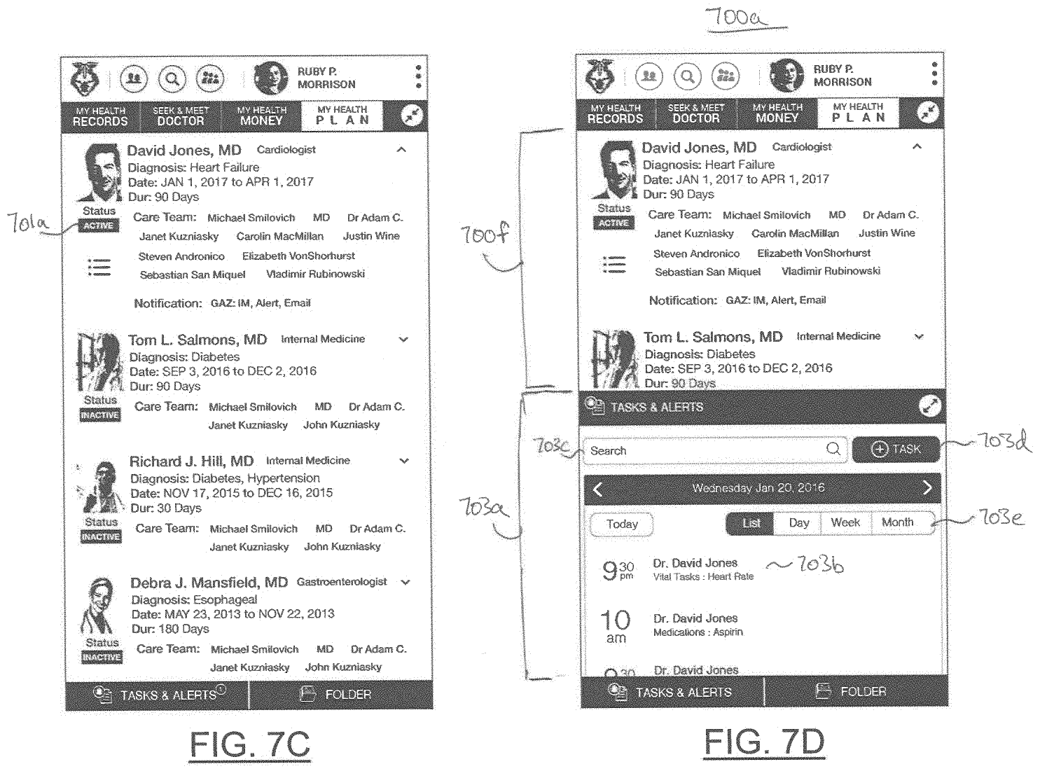

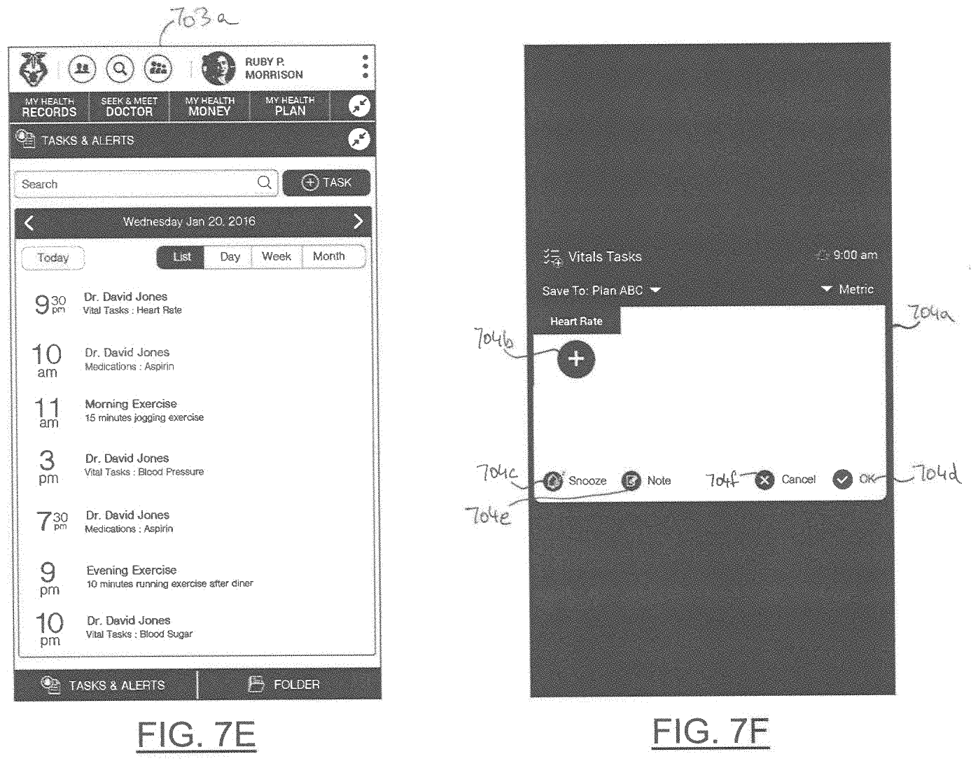

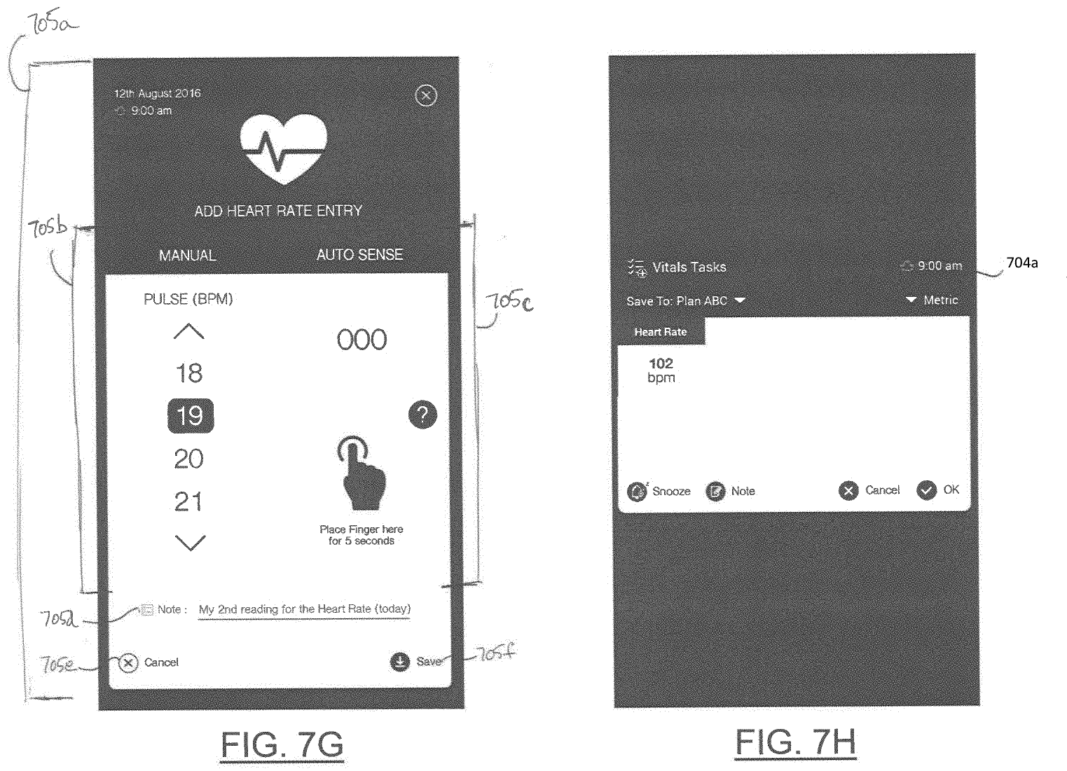

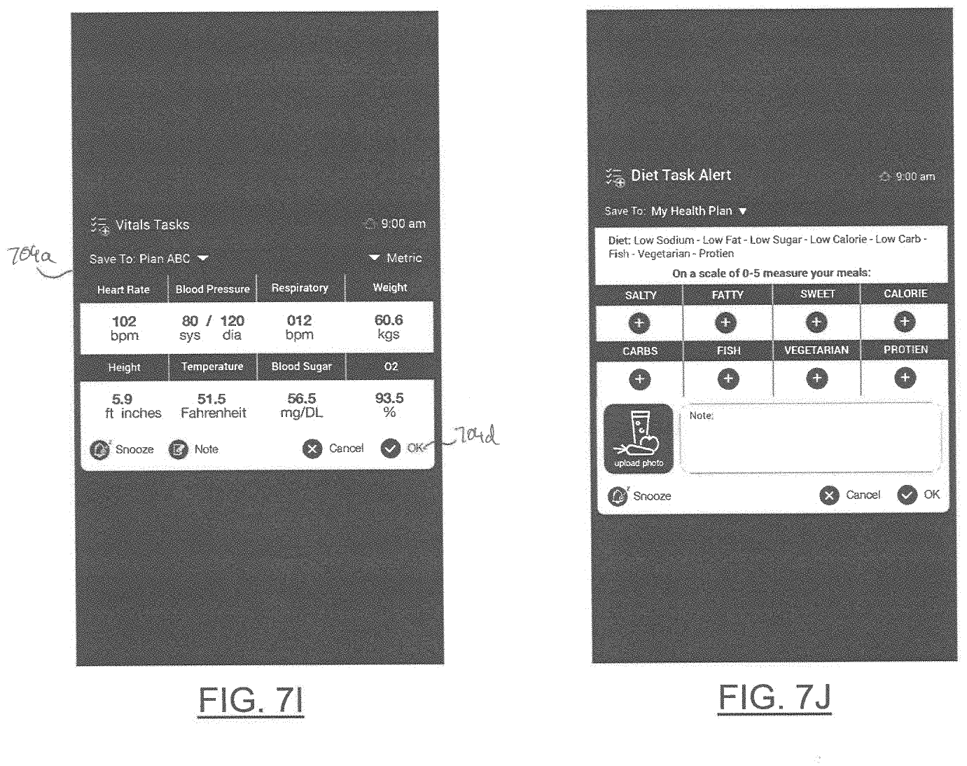

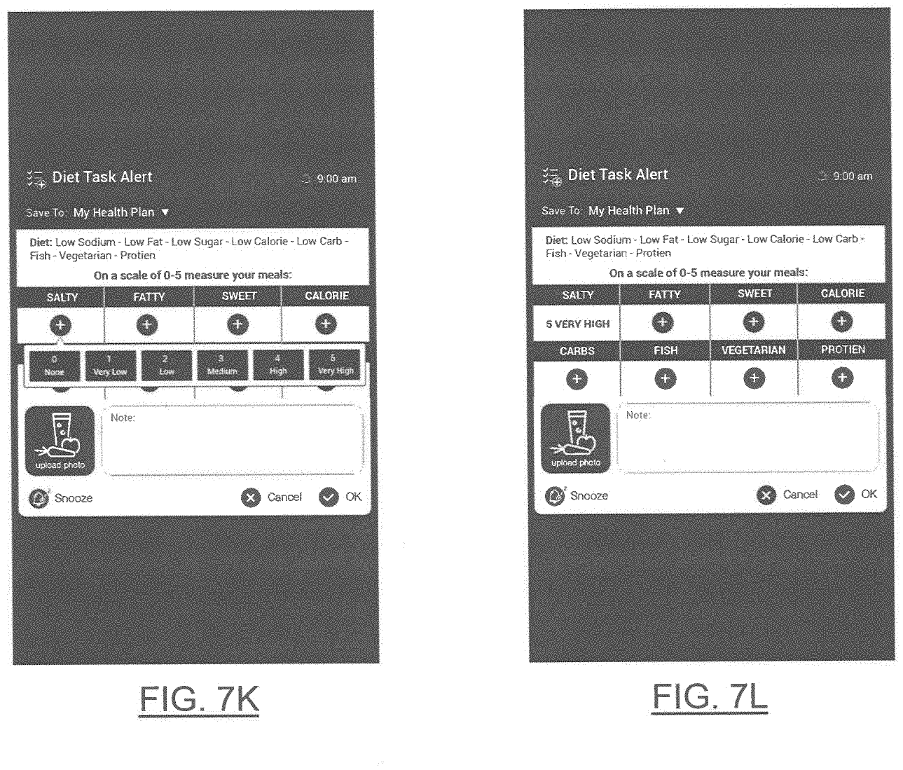

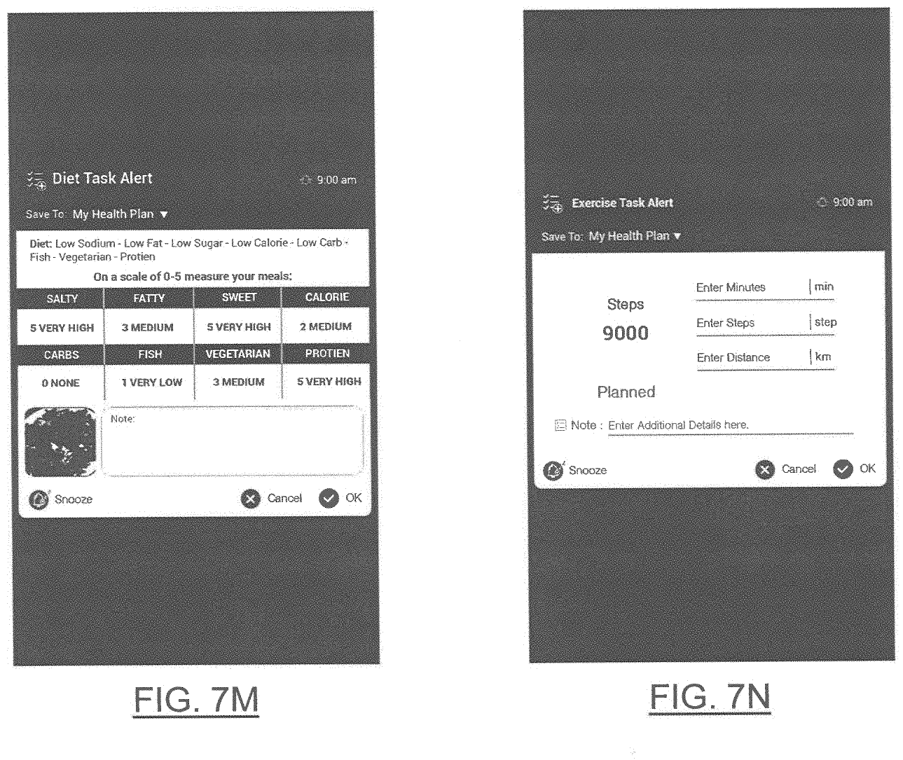

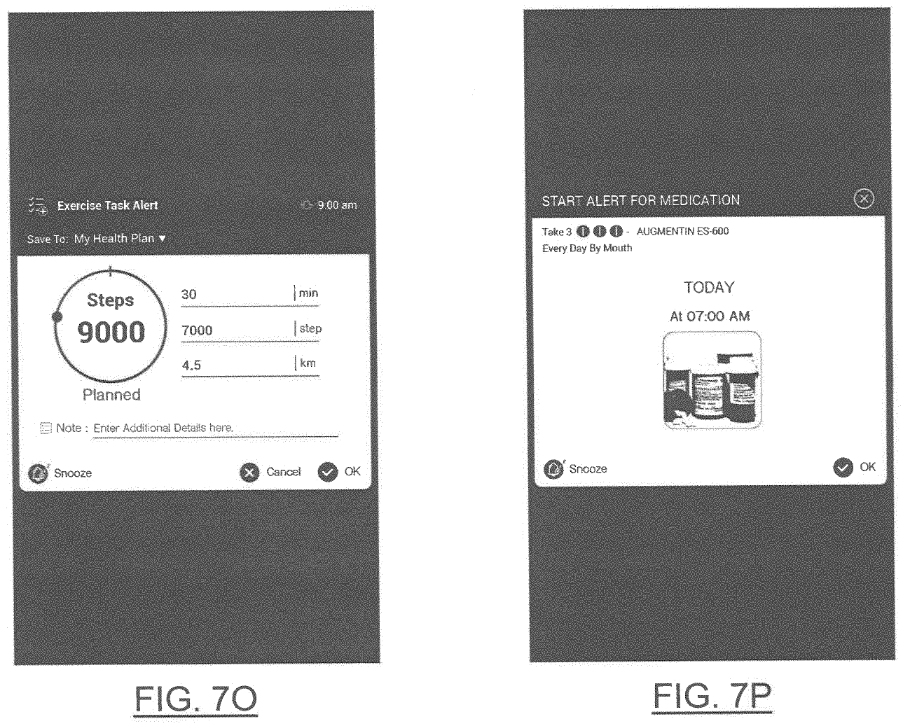

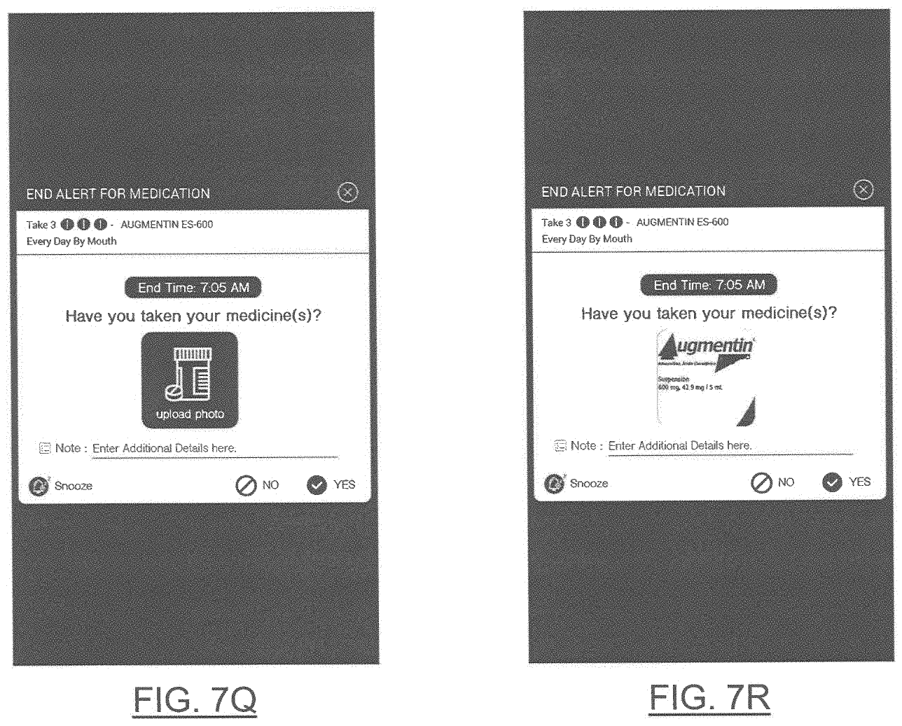

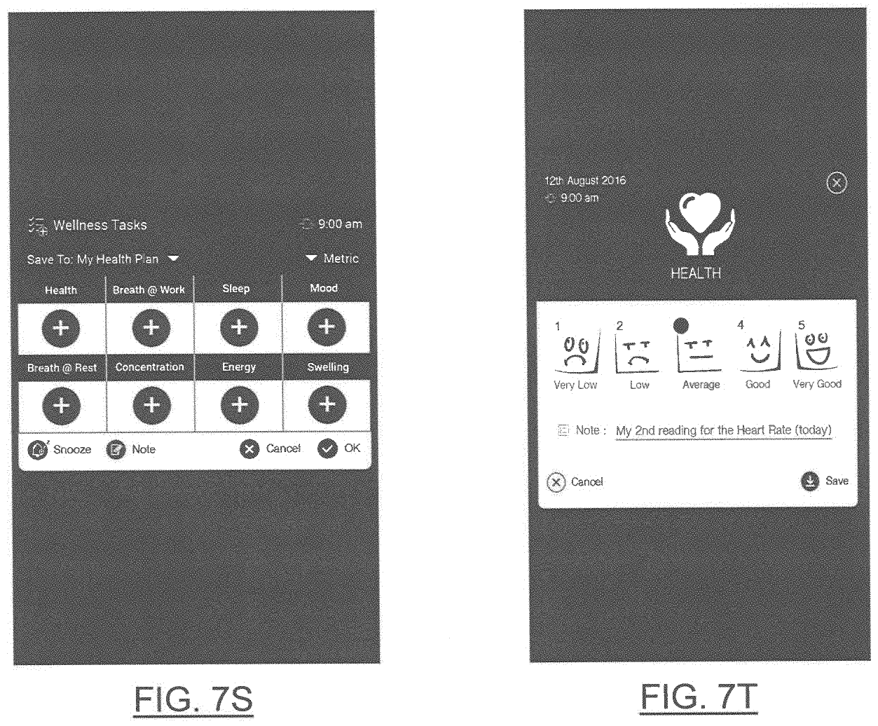

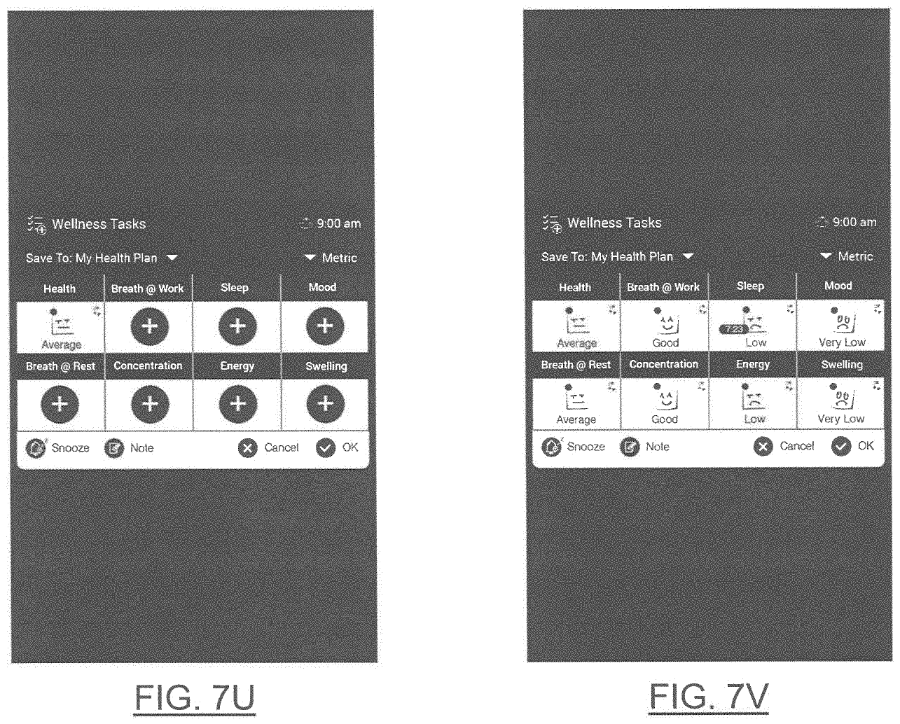

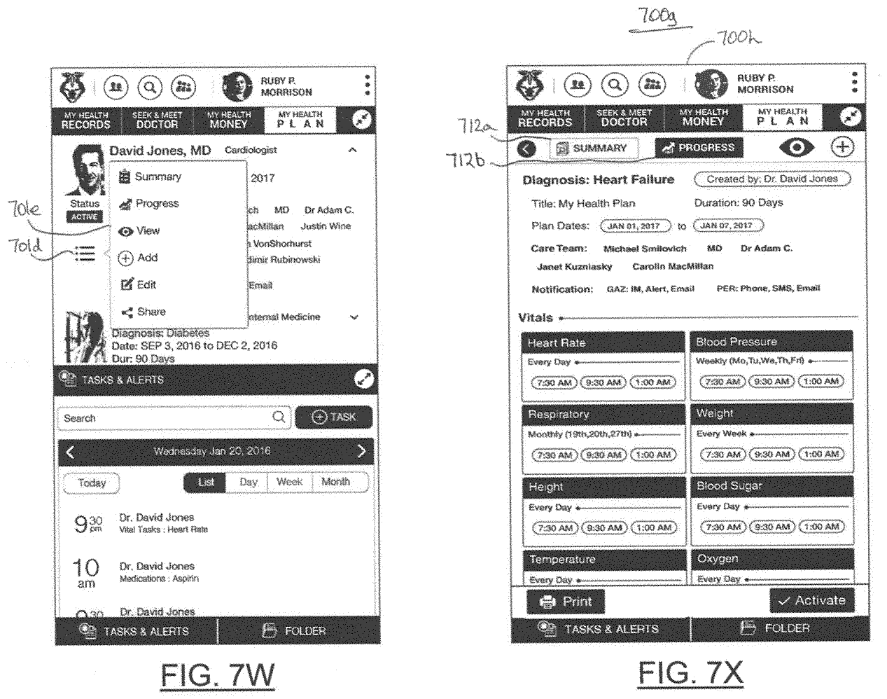

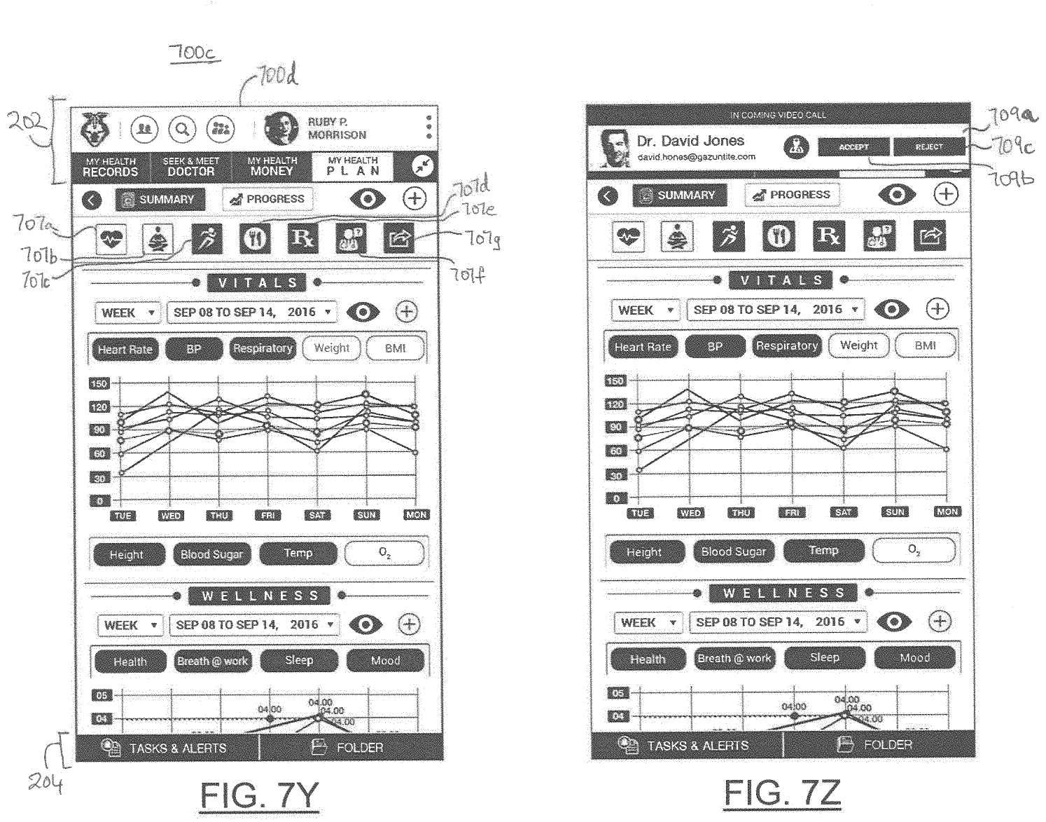

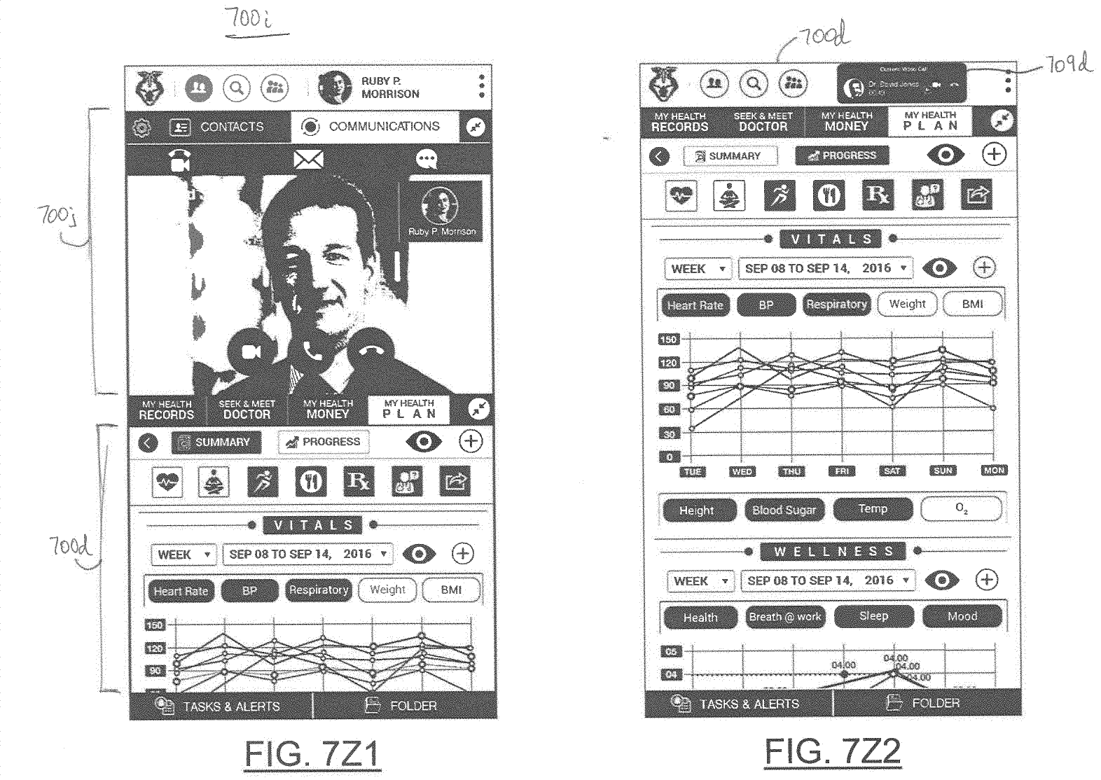

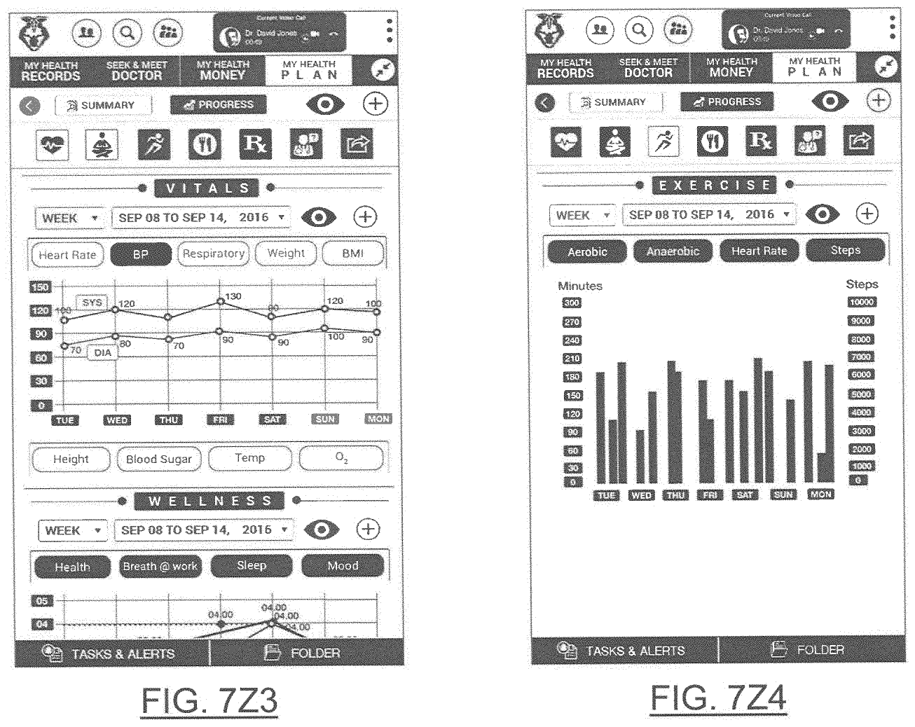

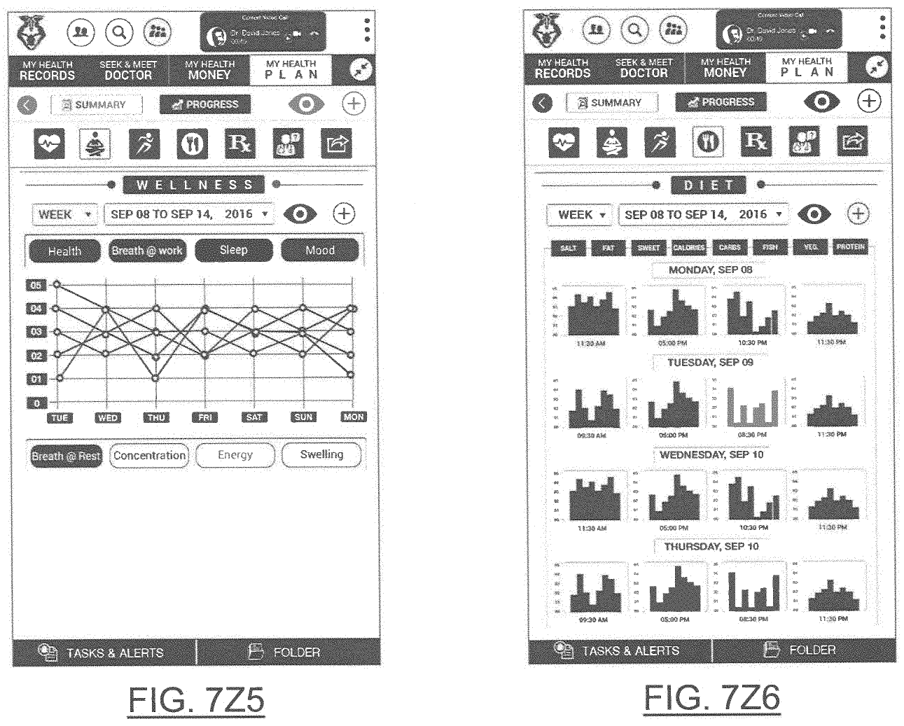

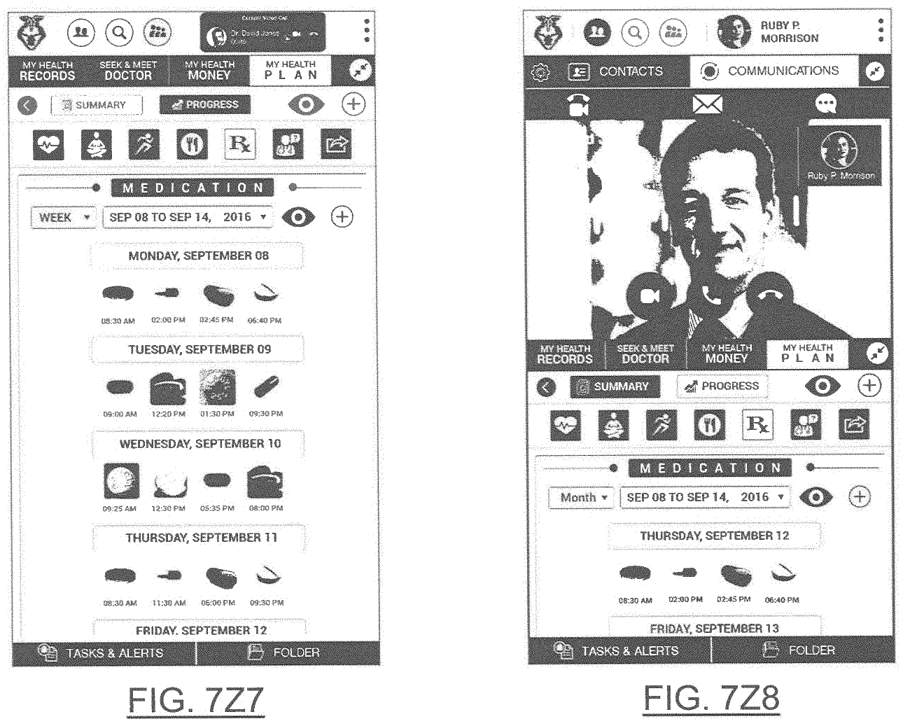

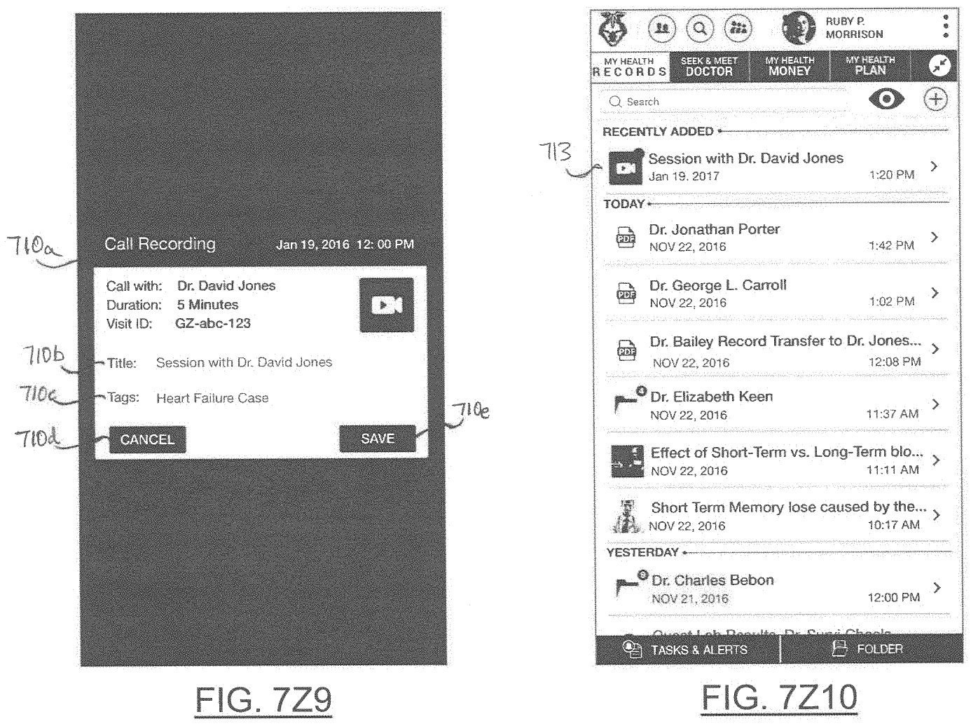

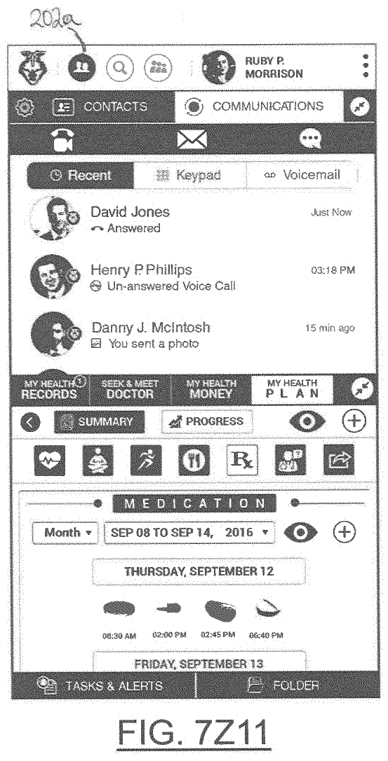

In response to the selection of the navigational graphical item 204a, the patient monitoring platform 101 is further configured to display an application screen with a GUI that displays one or more tasks assigned to the patient. In some embodiments, the patient monitoring platform 101 is operably coupled to a calendar application associated with the user, such as the patient, and the patient monitoring platform 101 displays entries within the calendar application in response to the selection of the navigational graphical item 204a. Additional details of the tasks GUI are provided herein in the context of FIG. 7D. Similarly, navigational graphical item 204b is associated with an application screen and a GUI for displaying a list of folders and, in response to the selection of navigational graphical items 204b, the patient monitoring platform 101 presents the associated application screen and the GUI that displays the list of folders. In some embodiments, the patient monitoring platform 101 creates one or more default folders for every user of the patient monitoring platform 101 and includes the one or more default folders when displaying the list of folders. In some embodiments, the patient monitoring platform 101 automatically saves data saved by a user into one of the default folders if the user does not specify a particular folder to save the data. Additional details of creation of folders, contents of the folders, and displaying the list of folders are provided further herein in context of FIG. 6F.

The GUI 200c further includes notification graphical items 203a, 203b, 203c, 203d, collectively notification graphical items 203. Each of the notification graphical items 203 displays information related to alerts, tasks, reminders, and certain summarized information to be communicated to the user. For example, the notification graphical item 203a displays an alert from a care provider requesting to share a health record of the patient with another care provider and the patient monitoring platform 101 displays additional details regarding the request in response to selection of the notification graphical item 203a, including additional graphical items (not shown) configured to allow the user or patient to accept or reject the request. Similarly, the notification graphical item 203b displays a reminder of an appointment with a care provider, the notification graphical item 203c displays summarized information related to health expenses and/or health expenses related transactions of the patient, and the notification graphical item 203d displays task related to a care plan for the patient. In some embodiments, in response to selection of a notification graphical item 203, the patient monitoring platform 101 displays additional information related to the user. For example, selection of the notification graphical item 203b causes the patient monitoring platform 101 to display the location of the care provider, directions to the location of the care provider, and/or graphical items configured to accept a request for transportation to the location of the care provider from a transportation provider or service, such as a taxi service, etc. In some embodiments, the additional details of the task and/or alert displayed in the graphical item 203b. Similarly, in response to the selection of the notification graphical item 203c, the patient monitoring platform 101 displays additional and/or detailed information related to the health expenses and/or health expenses related transactions of the patient. In response to the selection of the notification graphical item 203d, the patient monitoring platform 101 displays additional and/or detailed information related to the care plan whose information is presented in a summarized form in the notification graphical item 203d.

One or more of the notification graphical items 203 may be associated with the same application screen and GUI as the application screen and GUI associated with one or more of the navigational graphical items 202, 204. For example, the patient monitoring platform 101 displays the application screen and the GUI associated with navigational graphical item 204a in response to the selection of the notification graphical item 203b. Similarly, in response to the selection of the notification graphical items 203a, 203c and 203d, the patient monitoring platform 101 displays the application screen and the GUI associated with navigational graphical items 202f, 202h and 202i, respectively.

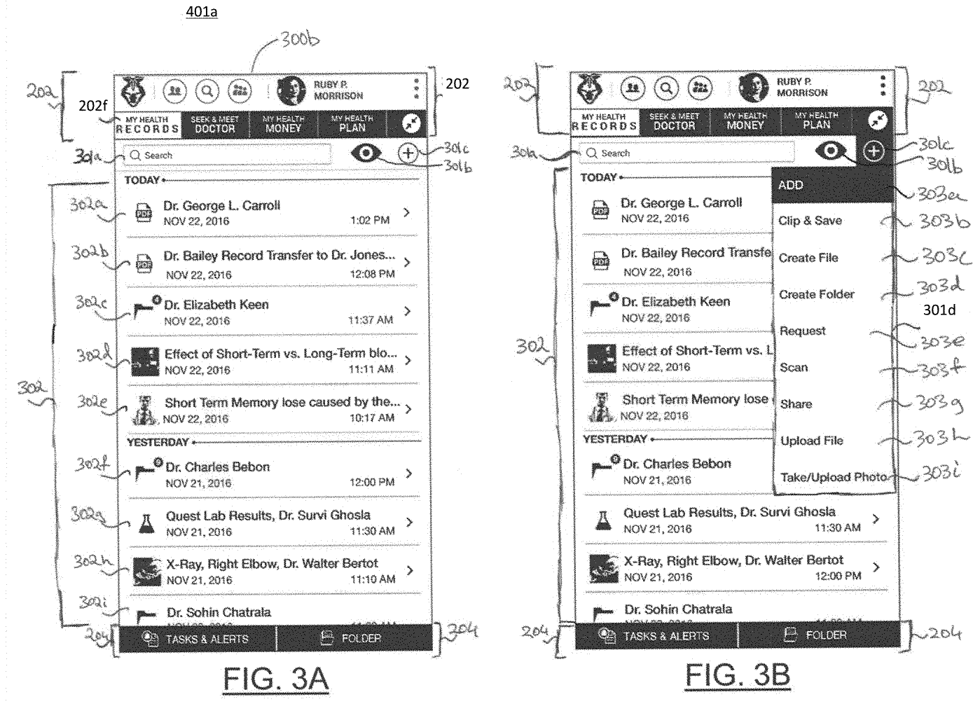

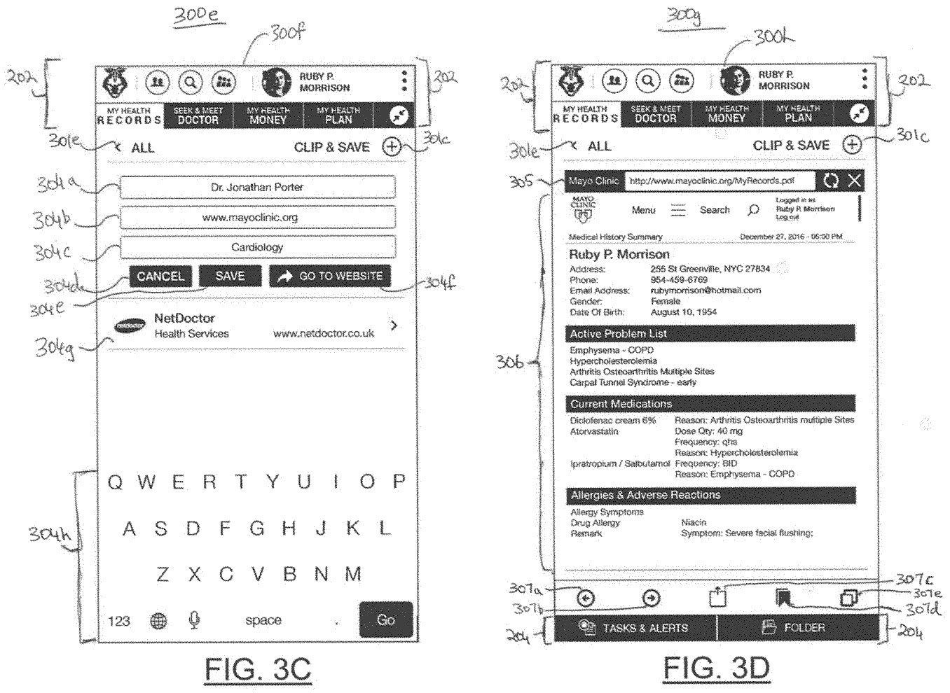

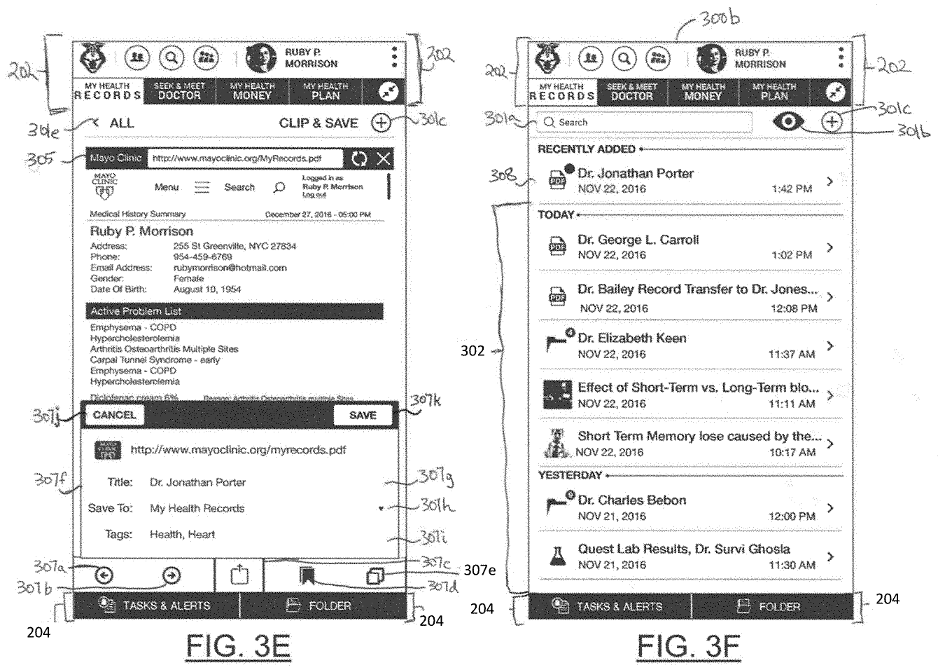

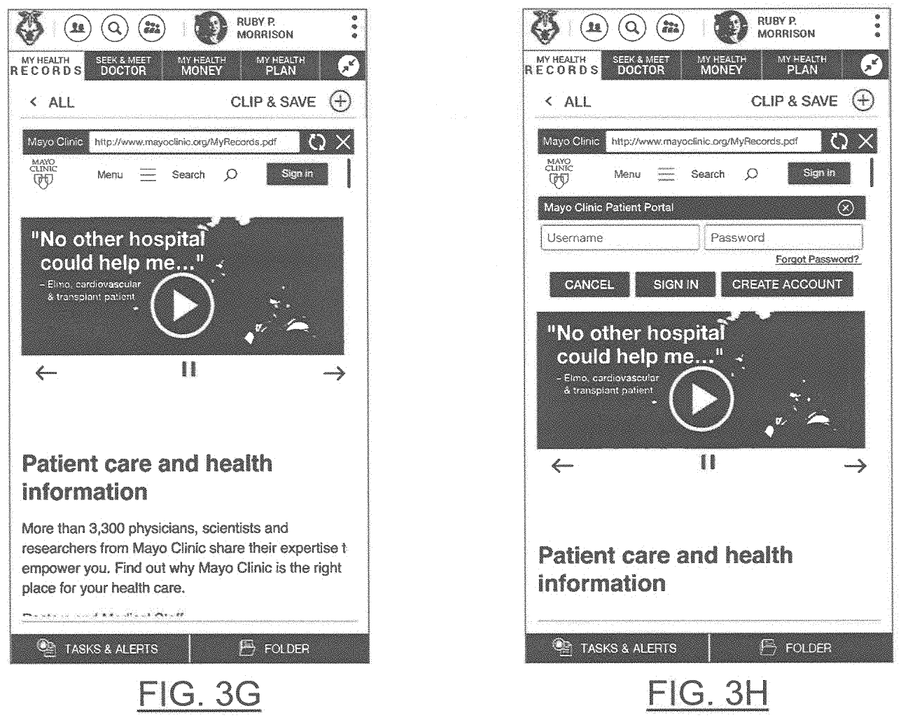

As described above, the patient monitoring platform 101 is configured to present an application screen displaying a GUI for viewing, uploading, and/or interacting with health records, labs, diagnostic images, or health-related files of a patient in response to the selection of navigational graphical item 202f. Application screen 300a and GUI 300b, shown in FIG. 3A, is an example of such an application screen and GUI for viewing, uploading, and/or interacting with health records and health-related data of the patient. The GUI 300b includes navigational graphical items 202 and 204. The GUI 300b further includes graphical items 301a, 301b, 302a, 302b, 302c, 302d, 302e, 302f, 302g, 302h, 302i (collectively graphical items 302), 303a. The graphical item 301a is configured to receive search terms and the patient monitoring platform 101 searches for health records, labs, diagnostic images, and/or health-related files associated with the patient and the search terms received by the graphical item 301a. In response to the selection of the navigational graphical item 301a, the patient monitoring platform 101 allows a user, such as the patient, to input search terms into the graphical item 301a and the patient monitoring platform 101 displays health records, labs, diagnostic images, and health-related files associated with the search terms and the user as graphical items in the GUI 300b, similar to the graphical items 302. The graphical items 302 are associated with data related to health records of the patient, health-related files, such as articles, saved by the patients, folders that include health records or links to health records of the patient, lab results of the patient, etc. In some embodiments, the patient monitoring platform 101, in response to the selection of the graphical item 202f, automatically displays graphical items 302 when displaying the GUI 300b.

The patient monitoring platform 101 displays the health records of the patient in multiple ways including in separate graphical items, such as graphical items 302a, 302b. The patient monitoring platform 101 displays the health records of the patient in detail in response to the selection of the graphical items 302a, 302b. The patient monitoring platform 101 may associate a health record of the patient with the care provider of the patient. For example, as shown by graphical item 302a in FIG. 3A, one of the health records of the patient is associated with a care provider and another health record of the patient is associated with another care provider, as shown by graphical item 302b. In some embodiments, the patient monitoring platform 101 displays, within the graphical items 302a, 302b, summarized information about their respective care providers including images, such as an image related to a diagnosis performed by the care provider (not shown).

The patient monitoring platform 101 may also display folders that include health records of the patient in separate graphical items, such as graphical items 302c, 302f, 302i. In response to the selection of the graphical items 302c, 302f, or 302i, the patient monitoring platform 101 displays all the health records associated with the folders displayed in graphical items 302c, 302f, and 302i, respectively. In some embodiments, the patient monitoring platform 101 displays all files associated with the folders displayed in graphical items 302c, 302f, 302i, in response to the selection of the graphical items 302c, 302f, 302i, respectively. The patient monitoring platform 101 may also display health-related files, such as articles, etc., which are saved by the patient using the patient monitoring platform 101 in separate graphical items, such as graphical items 302d, 302e. The patient monitoring platform 101 displays the health-related file or additional details of the health-related file in response to the selection of the graphical items 302d or 302e. Similarly, the patient monitoring platform 101 displays the labs and diagnostic images in separate graphical items, such as graphical items 302g, 302h, and the patient monitoring platform 101 displays data related to the labs or the diagnostic images, in response to the selection of the graphical items 302g, 302h, respectively.

The graphical item 301b is associated with presenting different views of the health records presented by the patient monitoring platform 101, including presenting graphical items (not shown) to select particular dates or a range of dates and the patient monitoring platform 101 displays health records modified or saved on the selected dates. The graphical item 301c is associated with uploading digital files such as health records, labs, diagnostic images, any other health related file, etc. of the patient to the patient monitoring platform 101. The graphical item 301c is also associated with saving digital files, such as web articles, portable files, any other health-related file, etc., selected by the patient. The functionality of uploading and saving digital files associated with the graphical item 301c will be referred to herein as “clip-and-save.” In response to selection of the navigational graphical item 301c, the patient monitoring platform 101 presents graphical item 301d, as shown in FIG. 3B. In some embodiments, the graphical item 301d is a drop down list, as shown in FIG. 3B.

The graphical item 301d includes graphical items 303a, 303b, 303c, 303d, 303e, 303f, 303g, 303h, 303i. The graphical item 303a is associated with uploading a health record of the patient from a health portal to the patient monitoring platform 101. The patient monitoring platform 101 is configured to initiate the process of uploading a health record to the patient monitoring platform 101 and presents application screen 300e and GUI 300f, as shown in FIG. 3C, in response to the selection of the graphical item 303a. The GUI 300f includes navigational graphical items 202, graphical items 301c, 301e, 304a, 304b, 304c, 304d, 304e, 304f, 304g, 304h. The GUI 300f allows a user to provide information about a care provider of the patient that has a health record of the patient and includes graphical items 304a, 304b, 304c, which are configured to receive text input from the user about biographical information of the care provider (e.g., name of the care provider), information about a portal associated with the care provider through which patient can access his/her health record, professional information of the care provider (e.g., specialty of the care provider), respectively. In some embodiments, in response to the selection of any of the graphical items 304a, 304b, 304c, the patient monitoring platform 101 displays graphical item 304h. The graphical item 304h is a graphical keyboard configured to receive input for the various keys displayed and in response to the selection of a displayed key, the patient monitoring platform 101 enters the corresponding character in the graphical item that was selected. In some embodiments, the patient monitoring platform 101 displays the graphical item 304h without selection of any of the graphical items 304a, 304b, 304c, and displays characters corresponding to the selected displayed keys in whichever graphical item is selected. For example, if graphical item 304b is selected, then the patient monitoring platform 101 displays characters corresponding to the selected displayed keys in the graphical item 304b.

The patient monitoring platform 101 further allows the user to cancel the process of uploading the health record to the patient monitoring platform 101, save the progress of the process to complete at a later time, and continue with the process, through interaction with graphical items 304d, 304e, 304f, respectively. In some embodiments, the patient monitoring platform 101 displays a pop-up confirmation screen requesting a user confirmation for terminating the upload process in response to the selection of the graphical item 304d. The graphical item 301e is configured to receive input requesting displaying of all health records, labs, diagnostic images, and/or health-related files, as shown in FIG. 3A. In response to the selection of the graphical item 301e, the patient monitoring platform 101, displays the GUI shown in FIG. 3A, including new health records, labs, diagnostic images, and/or health-related files and previously displayed health records, labs, diagnostic images, and/or health-related files.

In response to the selection of the graphical item 304e, the patient monitoring platform 101 saves the data entered by the patient (or a non-patient user authorized by the patient), along with the association of each data entry with the respective graphical item. For example, in response to the selection of the graphical item 304e, the patient monitoring platform saves, in a data storage unit operably coupled to the patient monitoring platform 101, the data entered in the graphical item 304a along with an indicator that the data entered in the graphical item 304a is the name of the care provider and the data is to be associated with the graphical item 304a when the saved process of uploading the health record is resumed by the patient at a later time. The patient monitoring platform 101 further saves a link to the saved process and displays the link (not shown) to the saved process in a graphical item (not shown) and includes the graphical item with the GUI 300f. The patient monitoring platform 101 further associates the saved data and the link with the profile of the patient and/or the user that entered the data in the graphical items 304a, 304b, 304c. The patient monitoring platform 101 saves information associated with a health record that a user, such as a patient, has previously uploaded to the patient monitoring platform 101. The saved information associated with the health record includes, but is not limited to, the care provider biographical information, health portal information, the specialty of the care provider, etc. The patient monitoring platform 101 creates and displays a link to the health portal associated with a health record previously uploaded to the patient monitoring platform 101 in a graphical item, such as the graphical item 304g. In response to the selection of the graphical item 304g, the patient monitoring platform 101 displays a webpage of the health portal associated with the health record.

After entering the information of the care provider and the health portal, the patient (or a non-patient user authorized by the patient) may view his/her health record directly within the patient monitoring platform 101 by selecting the graphical item 304f In response to the selection of the graphical item 304f, the patient monitoring platform 101 presents application screen 300g and GUI 300h as shown in FIG. 3D. The GUI 300h includes navigational graphical items 202, 204, and graphical items 305, 306, 307a, 307b, 307c, 307d, 307e. The patient monitoring platform 101 is configured with and/or includes a web browser and the patient monitoring platform 101 is configured to execute and display the web browser in a GUI that is configured to display websites or web pages, such as the GUI 300h. The GUI 300h includes the web browser and the graphical items 305, 306, 307a, 307b, 307c, 307d, 307e are part of the web browser. The graphical item 305 includes a text box, configured to receive and display text, such as text related to the health portal's information, including a web address or a Uniform Resource Locator (URL) of the health portal. In some embodiments, the patient monitoring platform 101 displays the health portal information entered in the graphical item 304b in the text box of the graphical item 305. The GUI 300h further displays a web page or a web page of a website, such as the website of the health portal entered in the graphical item 304b, in the graphical item 306. In FIG. 3D, the graphical item 306 displays the web page that presents the health record of the patient. In some embodiments, a login page or a login prompt to the website associated with the health portal is presented in response to the selection of the graphical item 304f. An example of a GUI displaying the login page or the login prompt to the website associated with the health portal is shown in FIG. 3G and FIG. 3H, and the webpage displayed in the graphical item 306 (shown in FIG. 3D) is presented in response to authentication of the login credentials entered by the user.

Referring back to FIG. 3D, the graphical item 307a is associated with navigating to a previous web page from the currently displayed web page and the patient monitoring platform 101 displays the web page displayed before the currently displayed web page in response to the selection of the graphical item 307a. Similarly the graphical item 307b is associated with navigating to the next web page viewed after the current web page and patient monitoring platform 101, in response to the selection of the graphical item 307b, displays the web page displayed after the current web page was displayed. The graphical item 307d is associated with bookmarking the currently displayed web page and in response to the selection of the graphical item 307d, the patient monitoring platform 101 stores the URL of the currently displayed webpage and displays a graphical item associated with the URL in a list of graphical items that are associated with URLs of other bookmarked web pages. The graphical item 307e is associated with opening a new tab (not shown) within the web browser included in the GUI 300h, and the patient monitoring platform 101 displays the new tab within the web browser in response to the selection of the graphical item 307e.

The graphical item 307c is associated with the clip-and-save functionality of the patient monitoring platform 101, and in response to selection of the graphical item 307c, the patient monitoring platform 101 initiates the process of saving the health record displayed in the graphical item 306 to the patient monitoring platform 101, and displays the graphical item 307f, as shown in FIG. 3E. The graphical item 307f is a pop-up graphical item and includes one or more text boxes or drop down boxes, such as graphical items 307g, 307h, 307i, 307j, 307k, to allow the user, such as a patient, to enter information to associate with the health record and to save the health record displayed in the graphical item 306. As shown in FIG. 3E, the graphical item 307g includes a text box for entering a title of the health record, the graphical item 307h includes a drop down box to select a destination folder to save the health record, and the graphical item 307i includes a text box to allow the patient (or a non-patient user authorized by the patient) to enter one or more tags to associate with the health record. The patient monitoring platform 101 is configured to identify and retrieve a health record from a data storage unit storing the health record, if the patient, using the search functionality of the patient monitoring platform 101, enters the tag terms associated with the health record to search for the health record. The patient monitoring platform 101 closes the pop-up graphical item 307f in response to the selection of the graphical item 307j and saves the health record in a data storage unit operably coupled to the patient monitoring platform 101 and saves a graphical link to the health record within the destination folder specified in the graphical item 307h in response to the selection of the graphical item 307k. After saving the health record, the patient monitoring platform 101 also displays a graphical link to the health record in the GUI displaying all health records of the patient, such as the graphical item 308 included in the GUI 300b, as shown in FIG. 3F.

Referring back to FIG. 3B, the graphical item 303b is associated with the clip-and-save functionality, described above, and in response to the selection of the graphical item 303b, the patient monitoring platform 101 displays application screens and GUIs that include graphical items that allow users to search for information and save the searched information. In response to selecting a graphical item configured to receive input for saving the information, the patient monitoring platform 101 saves the information and associates the saved information with the profile of the user. In some embodiments, the patient monitoring platform 101 displays a link to the saved information in a graphical icon, such as a thumbnail image of the saved information. The graphical item 303c is associated with creating a file and the patient monitoring platform 101 displays application screens and GUIs that include graphical items that allow users to create and save files, such as text files, etc., in response to the selection of the graphical item 303c. The graphical item 303d is associated with creating a folder and the patient monitoring platform 101 displays application screens and GUIs that include graphical items that allow users to create folders and save folders in response to the selection of the graphical item 303d.

The graphical item 303f is associated with creating, uploading, and/or saving portable digital files to the patient monitoring platform 101 and associating the saved files to the profile of the user, such as the patient. The patient monitoring platform 101 initiates the process of capturing images in response to the selection of the graphical item 303f and presents graphical items to capture an image of a physical file. The patient monitoring platform 101 transforms the captured image into a file in a portable format, such as Portable Document Format (PDF), and saves the file in the portable format into a storage unit operably coupled to the patient monitoring platform 101 and associates the file in a portable format with the profile of the user. The graphical item 303g is associated with sharing one or more files of the patient with another user of the patient monitoring platform 101, such as a care provider. In response to selection of the graphical item 303g, the patient monitoring platform 101 displays one or more graphical items (not shown) that are configured to receive input from the user, such as the patient. The graphical items allow the user to specify the files to share, a communication medium via which to share the files, and confirm the sharing of the files.

The graphical item 303h is associated with importing data from other computing devices and systems. In response to the selection of the navigational graphical item 303h, the patient monitoring platform 101 displays a GUI (not shown) that includes one or more graphical items that are configured to receive input related to the data to import including but, not limited to, source location from which to import the data and a destination folder into which to store the data. The patient monitoring platform 101 stores the imported data in a data storage unit operably coupled to the patient monitoring platform 101 and associates the imported data with the user's profile associated with the patient monitoring platform 101. In some embodiments, the patient monitoring platform 101 is configured to determine whether data imported from an external computing device and/or system is in a format compatible with the patient monitoring platform 101 and adjust the format of the data to be compatible with the patient monitoring platform 101. The graphical item 303i is associated with capturing images and uploading captured images. In response to the selection of the graphical item 303i, the patient monitoring platform 101 displays one or more captured images or stream of images, such as a video, and one or more graphical items (not shown) configured to receive input to initiate the process of image capturing. In response to the selection of the graphical items configured to receive input to initiate the process of image capturing, the patient monitoring platform 101 initiates the image capturing process of the computing device hosting the patient monitoring platform 101 and provides a user interface (not shown) to the patient to capture images and videos. An example of a computing device hosting the patient monitoring platform 101 is a mobile computing device equipped with an image capturing device and configured with software relevant for capturing images, and provides a GUI to capture images or video. The patient monitoring platform 101 saves the captured images in data storage unit operably coupled to the patient monitoring platform 101 and associates the captured images with the profile of the user.

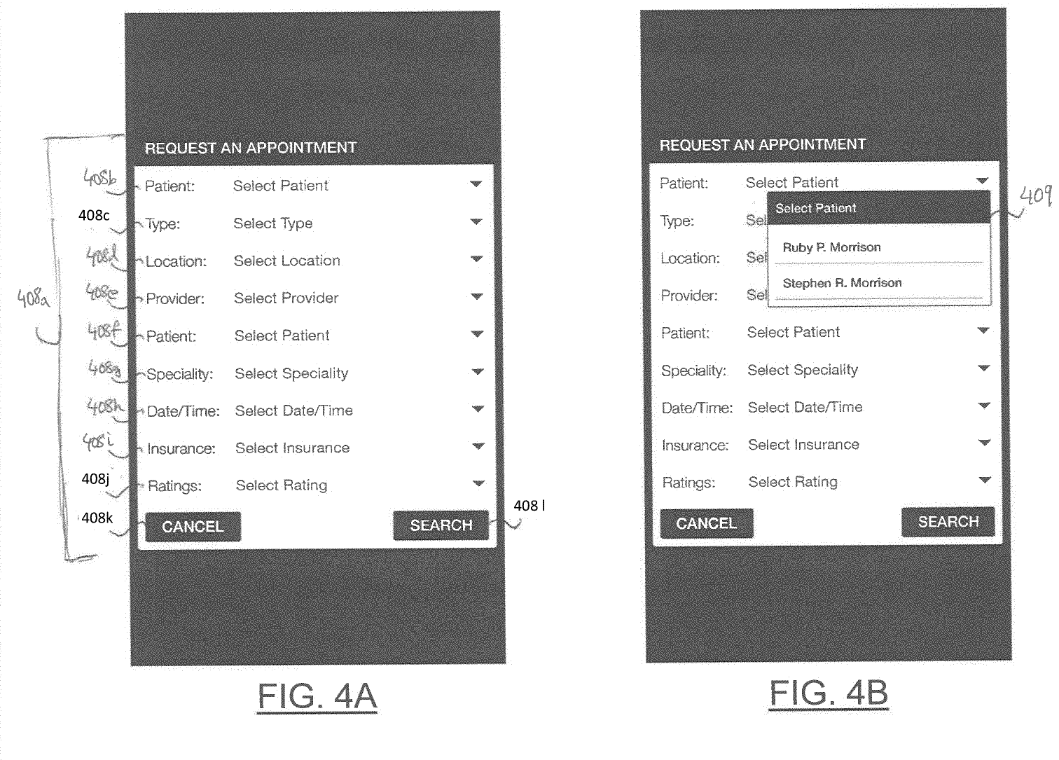

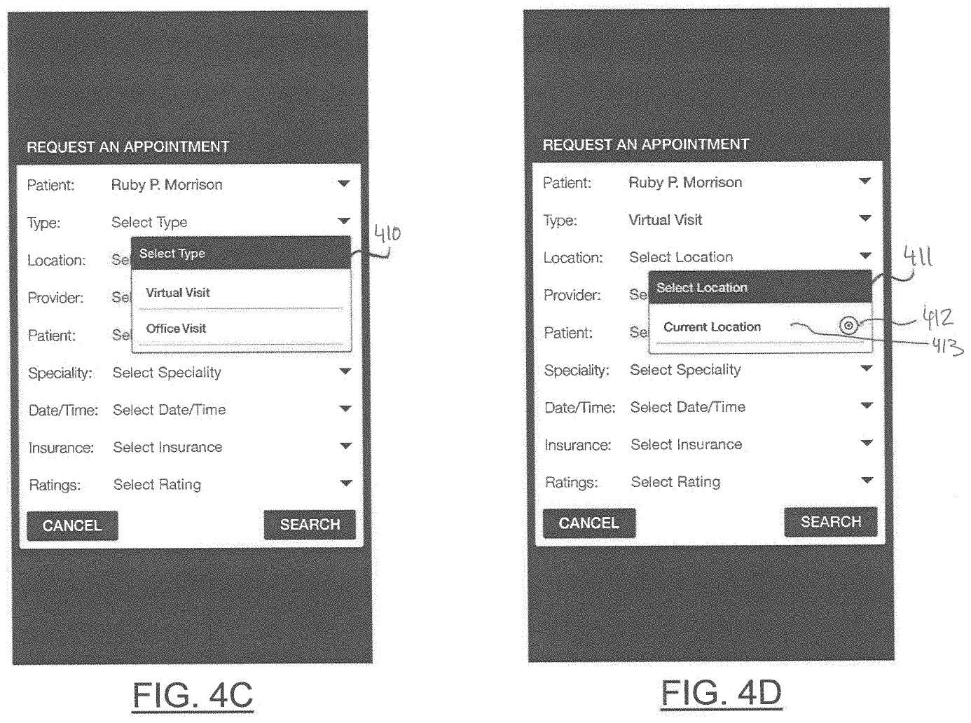

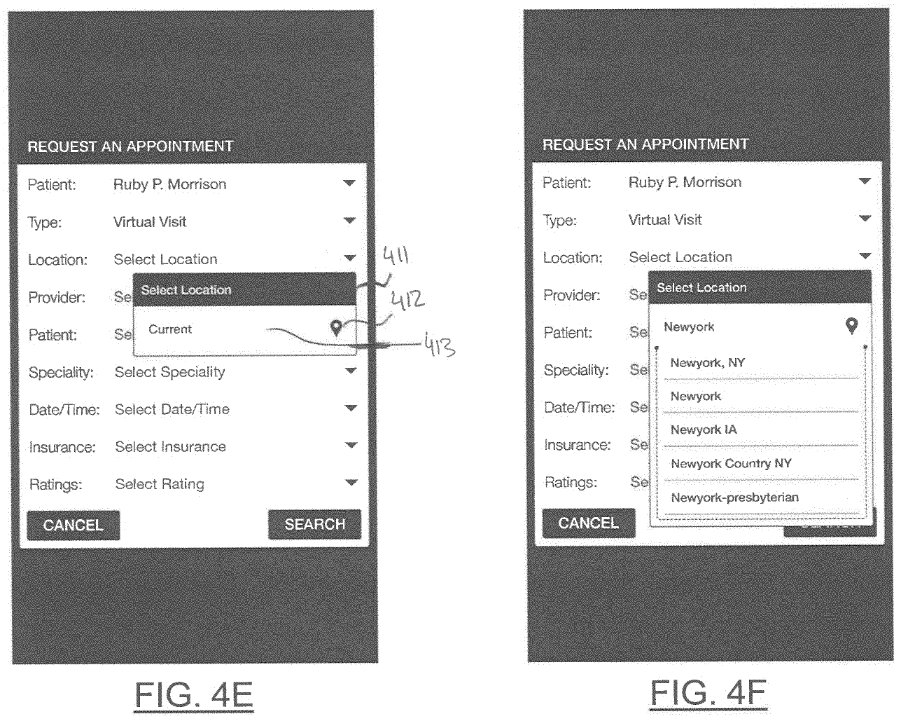

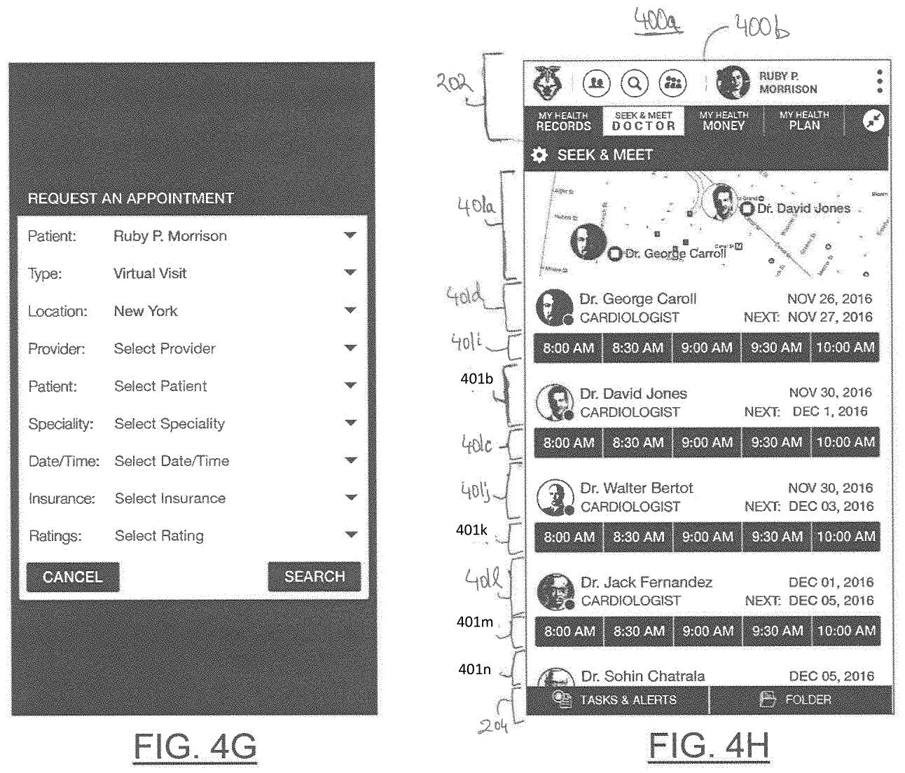

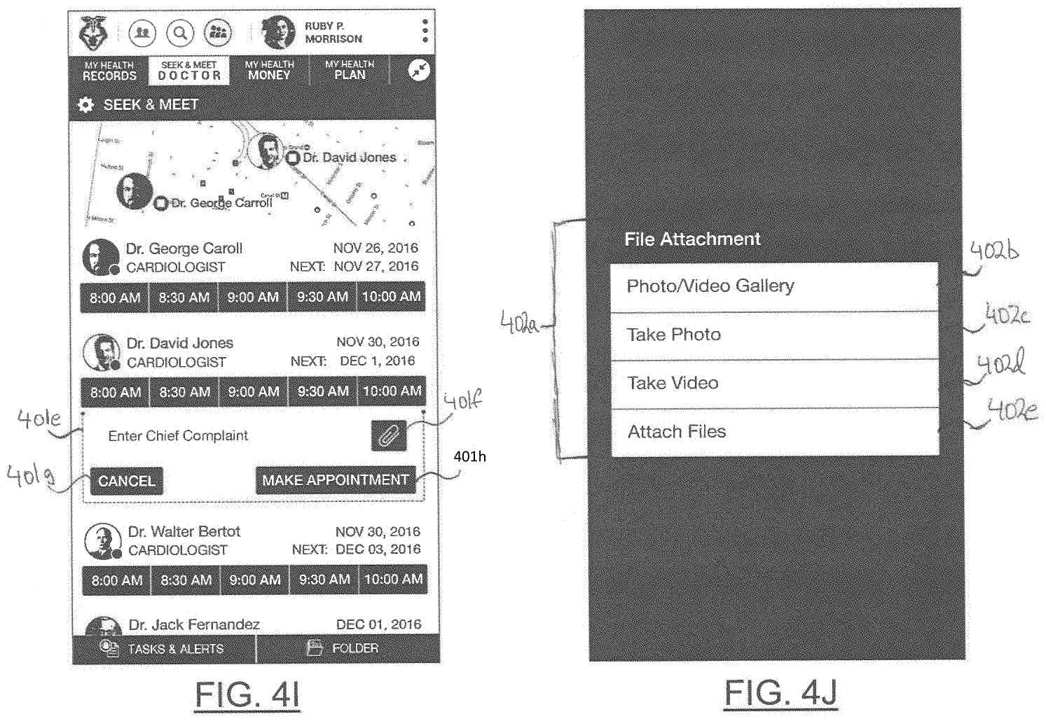

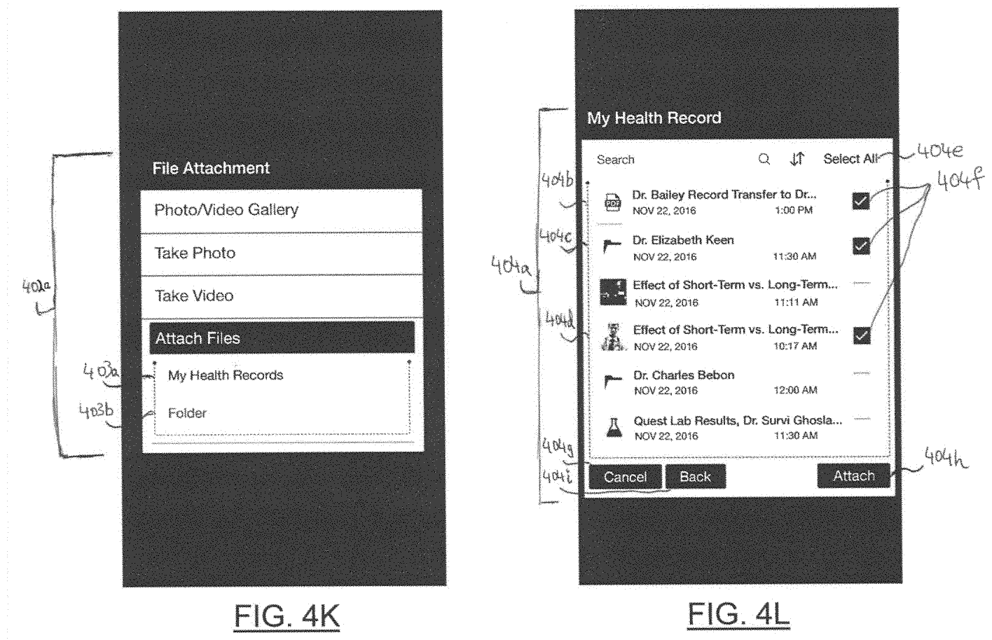

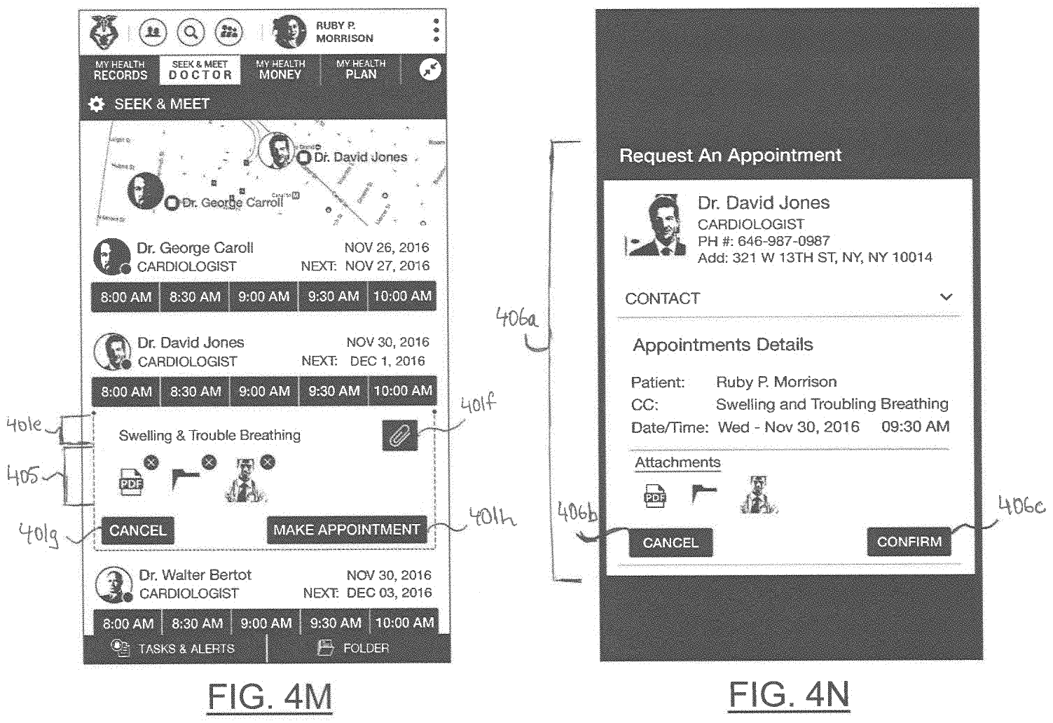

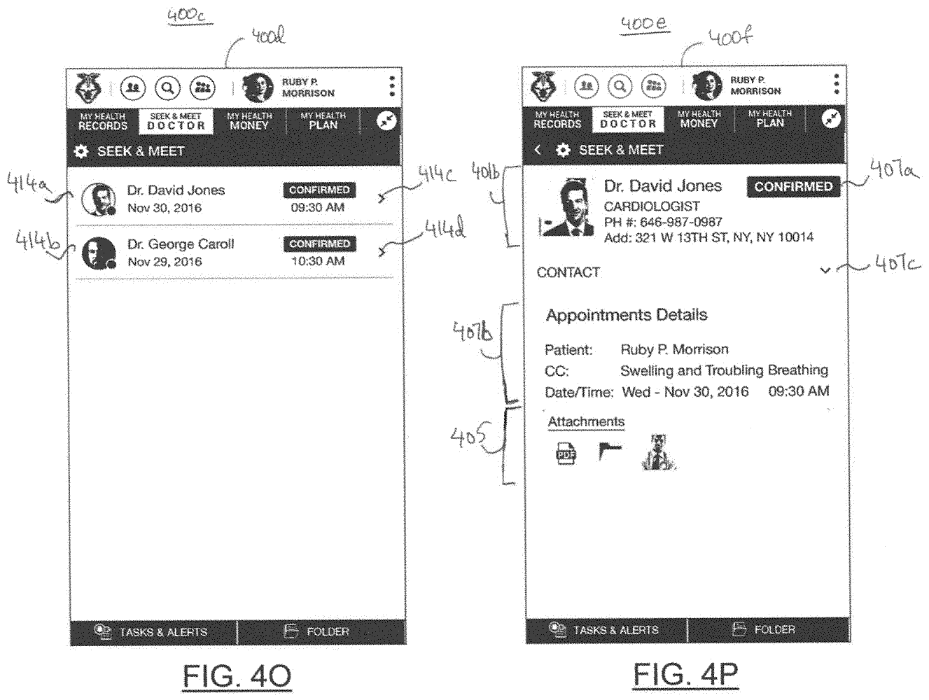

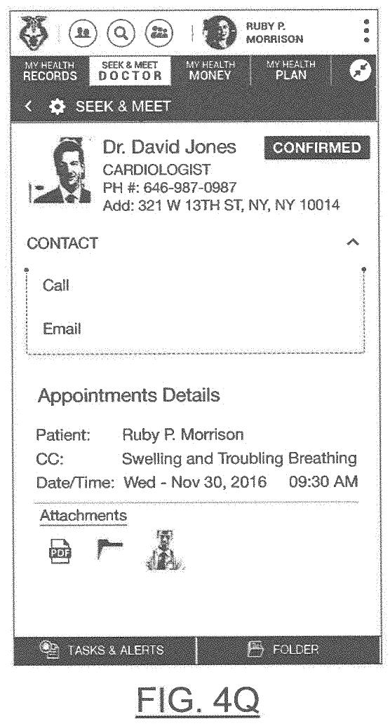

As noted above, the patient monitoring platform 101 is configured to present an application screen to allow a user to search for and interact with care providers (a functionality referred to herein as “seek-and-meet”) in response to the selection of the navigational graphical item 202g. Turning now to FIG. 4A, there is shown a graphical item 408a for searching for a care provider and scheduling an appointment with the care provider. The patient monitoring platform 101 displays the graphical item 408a, a pop-up graphical item, in response to the selection of the navigational graphical item 202g. The graphical item 408 includes graphical 408b, 408c, 408d, 408e, 408f, 408g, 408h, 408i, 408j, 408k, 408l, to search for care providers registered with the patient monitoring platform 101. The graphical item 408b is configured to receive an input to select a patient for whom an appointment with a care provider is sought. A user of the patient monitoring platform 101 may schedule appointments for him/herself and other users of the patient monitoring platform 101 that are part of the user's network. In response to selection of the graphical item 408b, the patient monitoring platform 101 displays a graphical item, such as graphical item 409, that presents other users within the network of the user, as shown in FIG. 4B. Upon selection of one of the users within the graphical item 409, the patient monitoring platform displays the selected user in the graphical item 408b, as shown in FIG. 4C.

The graphical item 408c is configured to receive an input for a type of visit with the care provider. The patient monitoring platform 101 is configured to allow users to receive medical services remotely by allowing users to interact with care providers via a communication medium, such as a video call, a voice call, a telephone call. In response to the selection of the graphical item 408d, the patient monitoring platform 101 displays a graphical item, such as graphical item 410, that displays types of visit with a care provider, an example of which is shown in FIG. 4C. Virtual visit indicates that the user wishes to receive medical services remotely and office visit indicates that the user wishes to receive medical services in-person. The patient monitoring platform 101 displays the selected visit in the graphical item 408c, as shown in FIG. 4D, in response to the selection of one of the types of visits.

The patient monitoring platform 101 displays graphical item 411, as shown in FIG. 4D, in response to the selection of the graphical item 408d, which is configured to receive an input for a location of the user. The patient monitoring platform 101 is configured to determine current location of the user in response to the selection of the graphical item 412, included within graphical item 411, as shown in FIG. 4D. In some embodiments, the patient monitoring platform 101 is configured to utilize location data gathered by the user's mobile computing device that is being used to access the patient monitoring platform 101, such as a smartphone, to determine the current location of the user. In some embodiments, the patient monitoring platform 101 is communicatively coupled to a global positioning system (GPS) and/or a system of a cell tower company configured to provide location data, and utilizes data received from these systems to determine current location. The patient monitoring platform 101 displays text in graphical item 413 (shown in FIG. 4D), included in graphical item 411, to communicate with the user that selection of the graphical item 412 initiates process of determining current location. In response to the selection of the graphical item 412, the patient monitoring platform 101 displays text that communicates with the user that current location has been selected for the location of the user for whom the appointment is being sought, an example of which is shown in FIG. 4E. The patient monitoring platform 101 also allows a user to enter location, as shown in FIG. 4F. The patient monitoring platform 101.

The graphical items 408e, 408g, 408h, 408i, 408j, are configured to receive inputs for a particular care provider that the patient seeks to schedule an appointment with, a specialty of care providers that the patient is interested in, a date and time for the appointment, a health insurance provider of the patient, and a minimum rating of the care providers, respectively. The patient monitoring platform 101 allows users to search for care providers without providing inputs for each of the graphical items 408e, 408g, 408h, 408i, 408j, as shown in FIG. 4G. In response to selection of the graphical item 408k, the patient monitoring platform 101 closes the pop-up graphical item 408a, and in response to the selection of the graphical item 408l, the patient monitoring platform 101 performs the search for care providers based on the user's inputs received by graphical 408a and presents an application screen 400a displaying a GUI 400b, as shown in FIG. 4H, to present the search results.

Turning now to FIG. 4H, there is shown the application screen 400a and the GUI 400b for selecting a care provider from among the care providers displayed and scheduling an appointment with the care provider. In some embodiments, the patient monitoring platform 101 orders the search results in a particular order, such as, by geographical distance from the location of the patient. The GUI 400b includes graphical items 401a, 401b, 401c, 401d. The graphical item 401a displays an interactive map displaying the locations of the care providers from the search results. For example, the search results for care providers includes Dr. David Jones and Dr. George Carroll and the graphical item 401a is displaying the locations of their respective offices. The graphical item 401b displays additional information about one of the care providers within the search result set. The additional information that may be displayed in the graphical items 401b, 401c include, but are not limited to, an image, name, professional attributes, such as an area of specialty, phone number, time periods available for an appointment, and address of the care provider. In FIG. 4H, the graphical items 401b, 401c display information related to a care provider within the search results, Dr. David Jones. Similarly, graphical items 401d, 401i displays information related to another of the care providers within the search result, Dr. George Carroll; graphical items 401j, 401k, display information related to Dr. Walter Bertot; graphical items 401l, 401m, display information related to Dr. Jack Fernandez; graphical items 401h display information related to Dr. Sohin Chatrala.

For each care provider registered with the patient monitoring platform 101, the patient monitoring platform 101 stores and updates dates and times that the care provider is available to meet with patients, referred to herein as “availability data.” In some embodiments, the patient monitoring platform 101 updates the availability data of a care provider in response to receiving an input from the care provider to update the availability data. In some embodiments, the patient monitoring platform 101 is operably coupled to scheduling systems of the care providers and the patient monitoring platform 101 automatically retrieves data related to the data availability of the care provider. In some embodiments, the patient monitoring platform 101 automatically updates the availability data of a care provider in response to receiving an input indicating that the care provider's availability data has been updated within the scheduling system at the care provider's medical facility.

For each care provider within the search result set, the patient monitoring platform 101 further displays, within a graphical item, one or more dates and timings for each date that the care provider is available. The patient monitoring platform 101 may display such graphical item adjacent to the graphical item displaying the additional information about the care provider. Examples of such graphical items are the graphical items 401c, 401i, 401k, 401m. The graphical item 401c is displayed adjacent to the graphical item 401b, associated with Dr. David Jones, and the graphical item 401c displays one or more dates that the care provider Dr. David Jones is available. For each date that the care provider is available for, the graphical item 401c displays one or more times at which the care provider is available to meet the patient. Each time displayed by the graphical item 401c is selectable and in response to the selection of a time displayed in the graphical item 401c, the patient monitoring platform 101 displays graphical items 401e, 401f, 401g, 401h, as shown in FIG. 4I.

The graphical item 401e is a text box configured to receive text from a user, and the patient monitoring platform 101, via the graphical item 401e, allows the patient or a non-patient user authorized by the patient to describe physiological conditions experienced by the patient, such as discomfort experienced by the patient, and/or any other information related to the health of the patient, such as information related to any self-diagnosis performed by the patient, prior to making an appointment with or visiting the care provider. The patient monitoring platform 101, via the graphical item 401f, allows the patient or a non-patient user authorized by the patient to upload and eventually transmit to the care provider any data believed to be helpful to the care provider in diagnosing the patient including, but not limited to, health records, web pages saved by the patient, lab results, diagnostic images, other types of images, etc. In response to the selection of the graphical item 401f, the patient monitoring platform 101 displays graphical item 402a, as shown in FIG. 4J.

The graphical item 402a, further includes graphical items 402b, 402c, 402d, 402e. In some embodiments, the graphical item 402a is a pop-window or a pop-up graphical item, as shown in FIG. 4J. Each of the graphical items 402b, 402c, 402d, 402e is selectable. In response to the selection of the graphical item 402b, the patient monitoring platform 101 displays one or more captured images or stream of images, such as a video, for the patient transmit to the care provider. In response to the selection of the graphical item 402c or 402d, the patient monitoring platform 101 initiates the image capturing process of the computing device hosting the patient monitoring platform 101 and provides a user interface (not shown) to the patient to capture images and videos. An example of a computing device hosting the patient monitoring platform 101 is a mobile computing device equipped with an image capturing device and configured with software relevant for capturing images, and provides a GUI to capture images or video, such as a smartphone.

The patient monitoring platform 101, in response to the selection of the graphical item 402e, presents graphical items 403a, 403b, as shown in FIG. 4K, to allow users to select one or more digital files of the patient, such as health records, prescriptions of the patient, labs, images, etc. The graphical item 403a is associated with health records, labs, diagnostic images, and/or other health-related files of the patient and in response to the selection of the graphical item 403a, the patient monitoring platform 101 displays user interfaces to select one or more health records, such as graphical item 404a (shown in FIG. 4L). The graphical item 403b is associated with folders created and associated with the profile of the patient. In some embodiments, the patient monitoring platform 101 displays multiple graphical items and associates each of the graphical items with a type of digital file.

The graphical item 404a displays each of the health records, labs, diagnostic images, and/or other health-related files of the patient as separate graphical items, such as graphical items 404b, 404c, 404d, included in graphical item 404a. The graphical item 404e allows the patient or a non-patient user authorized by the patient to communicate with the patient monitoring platform 101 that he/she wishes to select all health records of the patient with one user interaction, and the patient monitoring platform 101, in response to the selection of the graphical item 404e, identifies all health records, labs, diagnostic images, and/or other health-related files associated with the patient and includes copies of all the health records, labs, diagnostic images, and/or other health-related files within the message that is to be transmitted as part of the appointment request to the care provider, when the graphical item 404h is selected. The graphical item 404f allows the patient to select individual health records. In some embodiments, the patient monitoring platform 101 allows the patient to select the particular health records by selecting the graphical items 404b, 404c, 404d, and the patient monitoring platform 101 identifies the selected health records and includes the selected health records within the message requesting an appointment with the care provider, when the graphical item 404h is selected. In some embodiments, the patient monitoring platform 101, in response to the selection of certain graphical items, displays additional graphical items indicating the successful selection of the graphical items, such as the check mark displayed in the graphical items 404f.

The patient may wish to view the graphical item 402a shown in FIG. 4K and selects the graphical item 404i, and the patient monitoring platform 101, in response to the selection of the graphical item 404i, displays graphical item 402a in place of the graphical item 404a. Similarly, if the patient wishes to cancel the process of the including digital files in the message requesting an appointment with the care provider and selects the graphical item 404g, as shown in FIG. 4L, and in response, the patient monitoring platform 101 closes the graphical item 404a and displays the GUI shown in FIG. 4I.

In response to the selection of the graphical item 404h, the patient monitoring platform 101, displays all of the selected digital files in a graphical item, such as graphical item 405, shown in FIG. 4M, to present a visual indication of all of the selected files. The patient may then finalize the message requesting appointment with the care provider and send the message by selecting the graphical item 401h. In response to the selection of the graphical item 401h, the patient monitoring platform 101 displays a visual summary of the message, including the selected files, as shown by the graphical item 406a in FIG. 4N. The patient monitoring platform 101 allows the patient to cancel the message by selecting the cancel button of the graphical item 406b and the patient monitoring platform 101 closes the graphical item 406a in response to the selection of the graphical item 406b. If satisfied with the message, the patient monitoring platform 101 allows the patient to transmit the message to the care provider by selecting the graphical item 406c. In response to the selection of the graphical item 406c, the patient monitoring platform 101 transmits the message to the care provider and presents application screen 400c displaying GUI 400d, as shown in FIG. 4O. The GUI includes graphical items 414a, 414b, 414c, 414d. The graphical items 414a, 414b, display appointments scheduled with care providers, along with information related to the appointments, such as time of the appointment, date of the appointment, the care providers, etc. The graphical items 414c, 414d, included in graphical items 414a and 414b, respectively, and associated with the appointments displayed in graphical items 414a and 414b, respectively, are configured to receive input to display more detailed information of the appointment. In response to the selection of the graphical items 414c or 414d, the patient monitoring platform 101 presents the detailed information of the appointment associated with selected graphical item. For example, the patient monitoring platform 101 presents application screen 400e displaying GUI 400f, which presents the detailed information of the appointment associated with the graphical item 414c, as shown in FIG. 4P.

As shown in FIG. 4P, the GUI 400f presents to the user, such as the patient, a detailed visual view of the request for appointment sent to the care provider and includes graphical items 401b, 405, 407a, 407b, 407c. The graphical item 407a displays a current status of the request for appointment with the care provider. The patient monitoring platform 101, in addition to transmitting the request message to the care provider, also generates a notification and/or alert indicating the request for the appointment and presents the notification and/or alert in a notification graphical item (not shown separately in FIG. 4P) to the care provider. If the care provider accepts the request for the appointment, the patient monitoring platform 101 updates the status of the appointment request to indicate the acceptance of the request by the care provider and updates the graphical item 407a to display the change in the status. For example, when the care provider accepts the request for the appointment, the patient monitoring platform updates the graphical item 407a to display “CONFIRMED,” as shown in FIG. 4P. Additionally, the patient monitoring platform 101 inserts an entry in the calendar application of the patient that is operably coupled to the patient monitoring platform 101. In some embodiments, the patient monitoring platform 101 generates a notification or alert indicating the acceptance of the appointment request and displays the notification or alert in a graphical item (not shown) to the patient. The graphical item 407b displays the appointment details and the graphical item 407c is configured to receive input to display communication modes to contact and communicate with the care provider. In response to the selection of the graphical item 407c, the patient monitoring platform 101 displays one or more graphical items displaying the communication modes to communicate with the care provider. An example of such a graphical item is shown in FIG. 4Q.

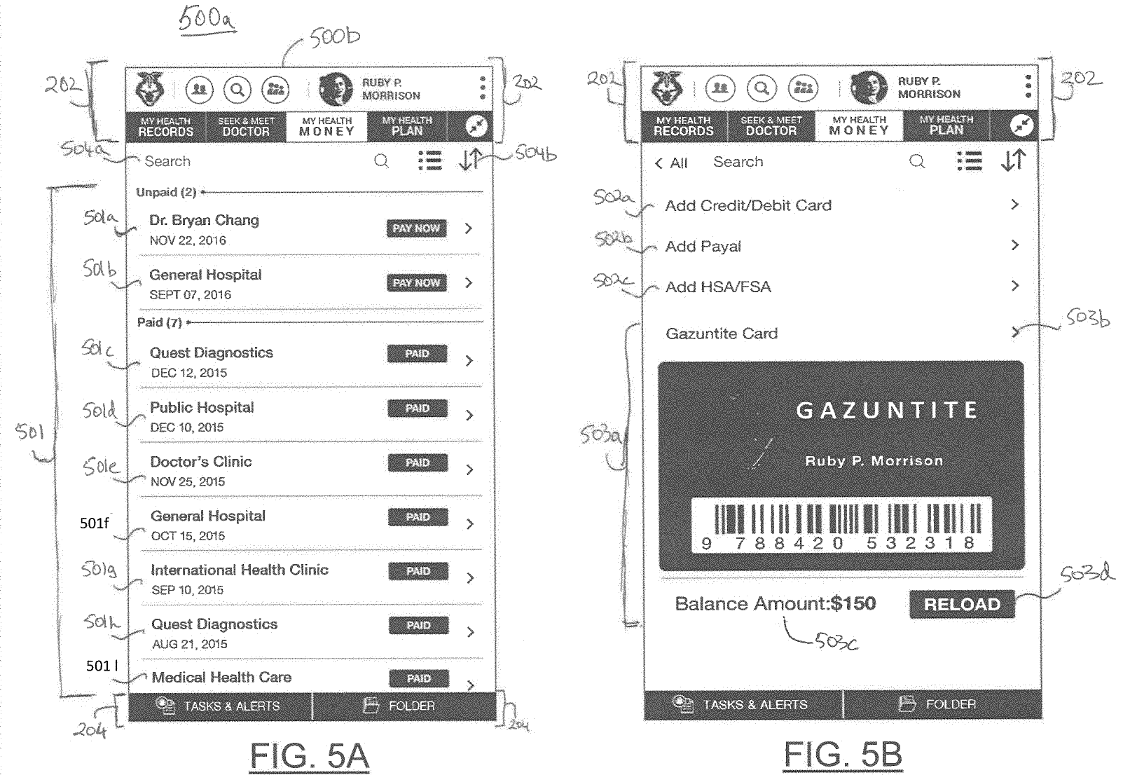

As described above, patients may manage payments using the patient monitoring platform 101, including payments to care providers, pharmacies, etc. The patient monitoring platform 101 is operably coupled to one or more payment processing systems and the patient monitoring platform 101 completes the financial transactions initiated using the patient monitoring platform 101 by transmitting the financial transaction to the payments processing systems. In response to the selection of the graphical item 202h, the patient monitoring platform 101 present application screen 500a displaying GUI 500b, as shown in FIG. 5A. Turning now to FIG. 5A, the GUI 500b includes navigational graphical items 202, 204, graphical items 501a, 501b, 501c, 501d, 501e, 501f, 501g, 501h, 501i, 504a, 504b. The GUI 500b displays a summary of all bills, pending and paid, of a user, such as the patient, in separate graphical items 501a, 501b, 501c, 501d, 501e, 501f, 501g, 501h, 501i, collectively graphical items 501. Each of the graphical items 501 is associated with the bill displayed by the graphical item. Each of the graphical items 501 displays information related to the bill, including, but not limited to, the care provider that generated the bill, date of the bill, a status indicator for the bill, etc. Each of the graphical items 501 is selectable and, in response to the selection of a graphical item 501, the patient monitoring platform 101 displays additional details for the bill associated with the selected graphical item. In some embodiments, the GUI 500b also groups the bills based on the status of the bills, as shown in FIG. 5A. In response to the selection of graphical item associated with a bill that has not yet been paid, the patient monitoring platform 101 displays an application screen and GUI for managing payments, including payment of an unpaid bill, using the patient monitoring platform 101. Application screen 500c and GUI 500d, shown in FIG. 5B, are examples of an application screen and a GUI for managing payments, including payments of unpaid bills, using the patient monitoring platform 101. In some embodiments, as part of the management of payments feature, the patient monitoring platform 101 allows the patient to view all financial accounts associated with the patient's profile on the patient monitoring platform 101 in one or more graphical items (not shown) for each financial account associated with the patient's profile, and, in response to the selection of the one or more graphical items, the patient monitoring platform 101 retrieves data related to the financial accounts associated with patient's profile from a data storage unit operably coupled to the patient monitoring platform 101 and presents the data in one or more additional graphical items (not shown). As used herein, the term “financial accounts” includes, but is not limited to, credit card accounts, debit card accounts, online payment accounts, mobile wallets, health savings accounts (HSA), flexible spending account (FSA). In some embodiments, the patient monitoring platform 101 stores data related to every financial transaction of the patient, and, the patient monitoring platform 101 retrieves and presents data related to financial transactions that the patient performed using the patient monitoring platform 101 in one or more graphical items (not shown) in response to the selection of a graphical item (not shown) included in the GUI 500b, which is configured to receive an input for displaying such transaction. In some embodiments, the patient monitoring platform 101 presents one or more graphical items to allow the patient to search for specific transactions, such as graphical item 504a, and/or sort, such as graphical item 504b, the presented financial transactions using date of the transaction occurred, amount involved the transaction, type of the transaction, etc.

Additionally, a patient may associate the patient's health insurance provider with the patient's profile on the patient monitoring platform 101 and the patient monitoring platform 101 is configured to save transactions between the patient and the health insurance provider. In some embodiments, the patient monitoring platform 101 presents the transactions between the patient and the health insurance provider when displaying the financial transactions. The patient monitoring platform 101 is configured to identify, from among the transactions between the health insurance provider and the patient, transactions that are reimbursements to the patient and displays those transactions in the GUI 500b in response to the selection of a graphical item configured to receive input for displaying such transactions. In some embodiments, the patient monitoring platform 101 is operably coupled to a transaction notification system of the health insurance provider and is configured to identify, from among the electronic transactions between the health insurance provider and the patient, transactions that are reimbursements from the health insurance provider to the patient and automatically update the reimbursement data presented in response to the selection of the graphical item configured to receive input for displaying reimbursements.

Turning now to FIG. 5B, the GUI 500d includes navigation graphical items 202, 204. The GUI 500d also includes graphical items 502a, 502b, 502c, 503a, 503b, 503c, 503d. The graphical items 502a, 502b, 502c allow the patient to add corresponding financial account. As shown in FIG. 5B, the graphical item 502a is associated with adding and associating with the patient's profile a credit card or debit card account, and in response to the selection of the graphical item 502a, the patient monitoring platform 101 initiates the process of adding a credit card and/or debit card account and displays one or more graphical items (not shown) to receive the necessary information to associate the patient's profile with the credit card or debit card account. Similarly, the graphical items 502b and 502c are associated with adding and associating with the patient's profile an online payment account and an HSA and/or FSA, respectively. The patient monitoring platform 101, in response to the selection of graphical item 502b or 502c, initiates the process of adding an online payment account and displays one or more graphical items (not shown) to receive all information necessary to associate the patient's profile with the online payment account or adding an HSA and/or FSA and displays one or more graphical items (not shown) to receive all information necessary to associate the patient's profile with the HSA and/or FSA, respectively.

The patient monitoring platform 101 allows the patient to select one financial account as a default financial account and the patient monitoring platform 101 is configured to use the default financial account prior to using any other financial account associated with the patient's profile. The patient monitoring platform 101 displays the default financial account and allows the patient to change the default financial account using one or more graphical items, such as the graphical items 503a, 503b. The graphical item 503a displays the default financial account of the patient and the patient may change the default financial account by selecting the graphical item 503b, which causes the patient monitoring platform 102 to display all the financial accounts associated with the patient's profile, and the default financial account is changed in response to the patient choosing a different financial account. The patient monitoring platform 101 is configured to display a balance of a financial account if available using a graphical item such as the graphical item 503c. For example, if the financial account is a debit card account or HSA or FSA, then the patient monitoring platform 101 displays the amount available in the respective accounts. Similarly, if the financial account is credit card account, the patient monitoring platform 101 may display the credit limit available for the patient on that credit card account. The patient monitoring platform 101 is configured to reload or transfer money into financial accounts that are configured to receive money. For example, in response to selection of the graphical item 503d, the patient monitoring platform 101 allows users to transfer money into the financial account displayed in the graphical item 503b. In some embodiments, in response to the selection of the graphical item 503d, the patient monitoring platform 101 displays one or more graphical items (not shown) to select a financial account into which the user desires to transfer money. In some embodiments, the patient monitoring platform 101 ranks or orders the financial accounts based on user inputs indicating a specific rank or order and the patient monitoring platform 101 traverses through the financial accounts based on the rank or order until a payment transaction is successfully completed.

In some embodiments, the patient monitoring platform 101 is configured to retrieve at least a portion of the information necessary to associate a financial account with a patient's profile using image capture and image analysis. As described above, the patient monitoring platform 101 is operably coupled to an image capturing device of the computing device being used to access the patient monitoring platform 101 and in response to the selection of a graphical item configured to receive an input to capture an image for retrieving financial account information, the patient monitoring platform 101 initiates the process of capturing images using the operably coupled image capturing device. The patient monitoring platform 101 is configured to recognize textual and numerical data in a captured image and extract the textual and numerical data from the captured image in response to a capture of the image. The patient monitoring platform 101 presents the user with a GUI (not shown) for controlling the image capturing device to include capturing images. Once an image is captured and if the image capture process is initiated in response to the selection of the graphical item configured to receive an input to capture an image for retrieving financial account information, the patient monitoring platform 101 identifies the numerical and textual information necessary to add a financial account from the captured image and displays a prompt or an alert to the user indicating the additional necessary information required, if any, to complete the addition and association of the financial account with the patient's profile.

After the meeting with the patient is completed, the care provider, using the patient monitoring platform 101, may initiate a billing process to generate a bill for the patient and transmit the bill to the patient (not shown separately in FIG. 5B). The patient monitoring platform 101 displays the transmitted bill as a notification and/or alert to the patient (not shown). The transmitted bill will be displayed in a graphical item in the GUI 500b, similar to the graphical items 501. In some embodiments, in response to the selection of the notification or alert, the patient monitoring platform 101 displays a prompt (not shown) to the patient asking whether the patient approves payment of the bill and in response to receiving a confirmation from the patient, the patient monitoring platform 101 initiates the generation of and transmits the payment transaction including the necessary information of the default financial account to be used in paying the bill.

The patient monitoring platform 101 is configured to allow a user to interact with multiple sub-applications or functionalities of the patient monitoring platform 101 simultaneously by presenting the GUIs for the multiple sub-applications or functionalities simultaneously on the display of the computing device being used to access an instance of the patient monitoring platform 101, such as the user computing device onto which an instance of the patient monitoring platform 101 is downloaded. The presentation of multiple sub-applications or functionalities simultaneously on the display will be referred to herein as “split screen.” The patient monitoring platform 101 splits screen in response to receiving an input indicating that a second sub-application or functionality has been selected while a first sub-application or functionality was being presented to the user. In some embodiments, the patient monitoring platform 101 splits screen based on a set of rules that indicate whether a sub-application or functionality may be displayed in a split screen or whether they may be displayed only in full screen and the patient monitoring platform 101 does not provide a split screen if the set of rules indicate that either the first or the second sub-application or functionality may be displayed only in full screen. In some embodiments, the patient monitoring platform 101, while displaying multiple GUIs in split screen, displays certain graphical items that are common to multiple GUIs, such as the navigational graphical items, outside of the GUI that includes it, for example, as shown in FIGS. 6A-6G, 7D, 7W, 7Z1, 7Z8, 7Z11, 9B, 11A, 11B, 11H, 11I.

In some embodiments, the set of rules indicate a location on the screen of the computing device being used to access the patient monitoring platform 101, near which to present the later selected sub-application or functionality and the patient monitoring platform 101 presents the later selected sub-application or functionality based on the rule indicating the location. For example, if the set of the rules indicate that the later selected sub-application or functionality is to be presented near the top of the screen, then the patient monitoring platform 101 displays the later selected sub-application or functionality near the top of the screen. An example of such a split screen by the patient monitoring platform 101 is shown in FIG. 6A described below.

In some embodiments, the patient monitoring platform 101 may be associated with configuration data that indicate a maximum number of sub-applications or figures that may be simultaneously presented and the patient monitoring platform 101 does not provide a split screen after the maximum number of sub-applications or functionalities are presented. For example, if configuration data associated with the patient monitoring platform 101 specifies that the maximum number of sub-applications or functionalities that may be presented is two, then the patient monitoring platform 101 does not split screen in response to the selection of the latest sub-application or functionality.

In some embodiments, the set of rules specify the sub-application that should be replaced if the patient monitoring platform 101 reaches the maximum number of sub-applications or functionalities that may be presented simultaneously. For example, if the maximum number of sub-applications or functionalities that may be simultaneously presented is 2 and the rules specify that the first sub-application or functionality should be replaced with the latest selected sub-application or functionality, then the patient monitoring platform 101 replaces the first sub-application or functionality with the latest selected sub-application or functionality if the patient monitoring platform 101 is already presenting two sub-applications or functionalities. An example of such a split screen by the patient monitoring platform 101 is shown in FIG. 6F described below. In some embodiments, the set of rules specify the location on the display to present the latest selected sub-application or functionality if the patient monitoring platform 101 reaches the maximum number of sub-applications or functionalities that may be presented simultaneously. For example, if the set of rules specify that the bottom portion of the display should be used in presenting the latest selecting sub-application and the maximum number of sub-applications or functionalities that may be displayed simultaneously is two, then the patient monitoring platform 101 replaces the sub-application or functionality at the bottom half of the screen if the patient monitoring platform 101 is already displaying two sub-applications or functionalities in split screen.

An example of split screen by the patient monitoring platform 101 is shown by application screen 600a and GUI 600b of FIG. 6A. The GUI 600b includes navigation graphical items 202, 204; GUI 500d, which includes navigation graphical item 503a; GUI 601a. As described above in FIG. 5B, the patient was presented with the manage payments functionality of the patient monitoring platform 101 and, as shown in FIG. 6A, the patient then selects the navigation graphical item 202b. As described above, the navigational graphical item 202b is associated with the search functionality of the patient monitoring platform 101 and in response to the selection of the navigational graphical item 202b, the patient monitoring platform 101 initiates the search functionality and executes and displays applications and GUIs associated with the search functionality. Examples of applications associated with the search functionality of the patient monitoring platform 101 include, but are not limited to, web browser. In some embodiments, the search functionality of the patient monitoring platform 101 is associated with a third-party search engine.

In response to the selection of the navigational graphical item 202b, the patient monitoring platform 101 initiates the process of split screen and displays the GUI 601a of a web browser of the patient monitoring platform 101 simultaneously with the GUI 500d of FIG. 5B, on the same display as the GUI 500d is being displayed. The GUI 601a includes graphical items 601b, 601c, 601d, 601e, 601f, 601g. The graphical item 601b is associated with navigating to a previously displayed webpage from the currently displayed webpage and the patient monitoring platform 101 displays the webpage displayed before the currently displayed webpage in response to the selection of the graphical item 601b. Similarly the graphical item 601c is associated with navigating to the web page viewed after the currently displayed web page and the patient monitoring platform 101, in response to the selection of the graphical item 601c, displays the web page displayed after the current web page was displayed. The graphical item 601g is configured to receive search terms for information the user wishes to search for and the patient monitoring platform 101 displays results related to the search terms in GUI 601a. In some embodiments, the patient monitoring platform 101 displays search suggestions, as shown in FIG. 6A, based on a user's activity with the patient monitoring platform 101 including the user's prior searches using the search functionality of the patient monitoring platform 101. In some embodiments, the patient monitoring platform 101 displays a search page of a search engine company and the user, such as the patient, may enter search terms in the appropriate text boxes of the search page. In some embodiments, the user may directly view a particular website by entering the URL of a website in the graphical item 601g.

In response to search terms being entered in the graphical item 601g, the patient monitoring platform 101 displays autocomplete suggestions for the search terms and/or website suggestions associated with the search terms. An example of the patient monitoring platform 101 presenting such suggestions is shown in FIG. 6B. In response to the patient monitoring platform 101 receiving an input to execute the search, the patient monitoring platform 101 displays the result set in the GUI 601a, including the websites associated with the search terms. In response to the selection of one of the search results, the patient monitoring platform 101 displays the associated webpage or website in the 601a, as shown in FIG. 6C. The graphical item 601e is associated with bookmarking the currently displayed web page and in response to the selection of the graphical item 601e, the patient monitoring platform 101 stores the URL of the currently displayed webpage and displays a graphical item associated with the URL in a list of graphical items that are associated with URLs of other bookmarked web pages. The graphical item 601f is associated with opening a new tab (not shown) within the web browser included in the GUI 601a, and the patient monitoring platform 101 displays the new tab within the web browser in response to the selection of the graphical item 601f.

The graphical item 601d is associated with the clip-and-save functionality of the patient monitoring platform 101 and in response to selection of the graphical item 601d, the patient monitoring platform 101 displays a graphical item that allows a user to save the webpage, such as graphical item 602a, as shown in FIG. 6D. The graphical item 602a includes graphical items 602b, 602c, 602d, 602e, 602f. The graphical items 602d and 602f are configured to receive text input from the user that specifies a title for the webpage and tags to associate with webpage, respectively. The graphical item 602e is configured to receive an input about a destination folder where access to the webpage will be displayed in a graphical item, such as a graphical icon.

The graphical item 602e is a drop down box that is configured to list all the folders associated with the patient's profile on the patient monitoring platform 101 and a destination folder is selected by the patient monitoring platform 101 based on the user selection of the one of the destination folders in the drop down list. The patient monitoring platform 101 closes the graphical item 602a in response to the selection of the graphical item 602b. And, in response to the selection of the graphical item 602c, the patient monitoring platform 101 saves web page in a data storage unit operably coupled to the patient monitoring platform 101 and displays a notification that the webpage has been saved in a graphical item, such as the graphical item 602g, as shown in FIG. 6E. In some embodiments the web page is saved in offline formats, including PDF. In some embodiments, the patient monitoring platform 101 displays the graphical item 602g and a GUI of the folders associated with the patient's profile including the selected destination folder and/or a copy of the URL in the selected destination folder. An example of such a GUI is GUI 603a, shown in FIG. 6F. As described above, FIG. 6F is an example where the configuration data of the patient monitoring platform 101 specify that the maximum number of sub-applications or functionalities that may be simultaneously presented or displayed is 2 and the rules specify that the first sub-application or functionality should be replaced with the latest selected sub-application or functionality. As shown in FIG. 6F, the patient monitoring platform 101 replaces the GUI of the first sub-application or functionality, the GUI 500d, with the GUI of the latest selected sub-application or functionality, the GUI 603a, by displaying the GUI 603a in place of the GUI 500d.

The GUI 603a is also associated with the navigation graphical item 204b and the patient monitoring platform 101 displays the GUI 603a in response to the selection of the navigation graphical item 204b. The GUI 603a includes graphical items 603b, 603c, 603d, 603e, 603f, 603h. The graphical item 603b is configured to receive text to allow a user to instruct the patient monitoring platform 101 to search for a digital file using search terms, such as file name, tags associated with the digital file, etc., provided in the graphical item 603b. The graphical item 603c is configured to receive input to initiate the process of the creating a folder and associating the folder to the user's profile by the patient monitoring platform 101, and the patient monitor platform 101 initiates the process of creating a folder and associates the folder to the user's profile, in response to selection of the graphical item 603c. The patient monitoring platform 101 displays all folders associated with the patient's profile in the GUI 603a. The graphical items 603d, 603e, 603f are associated with separate folders and the patient monitoring platform 101, in response to the selection of a graphical item among the graphical items 603d, 603e, 603f, 603h, presents one or more graphical items associated with the files associated with the corresponding folder, as shown in FIG. 6G. In FIG. 6G, the patient monitoring platform 101 displays the graphical item 603g in response to the selection of the graphical item 603f. The graphical item 603g is associated with the webpage saved in by the user in FIG. 6D and is configured to receive input to display the associated document. The patient monitoring platform 101 displays the document associated with the graphical item 603g, the webpage saved by the user in FIG. 6D, in response to the selection of the graphical item 603g. In some embodiments, the patient monitoring platform 101 displays the webpage as a standalone HyperText Markup Language (HTML) page. In some embodiments, the patient monitoring platform 101 executes and displays the web browser associated with the patient monitoring platform 101 and further displays the webpage in the web browser.

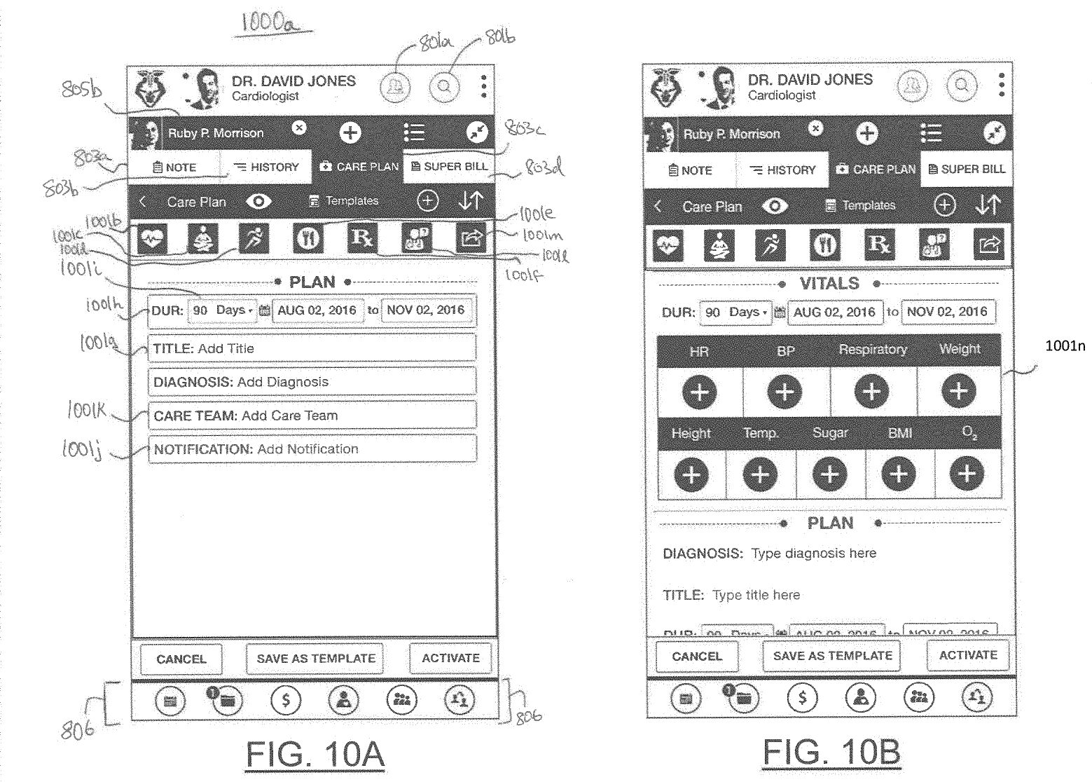

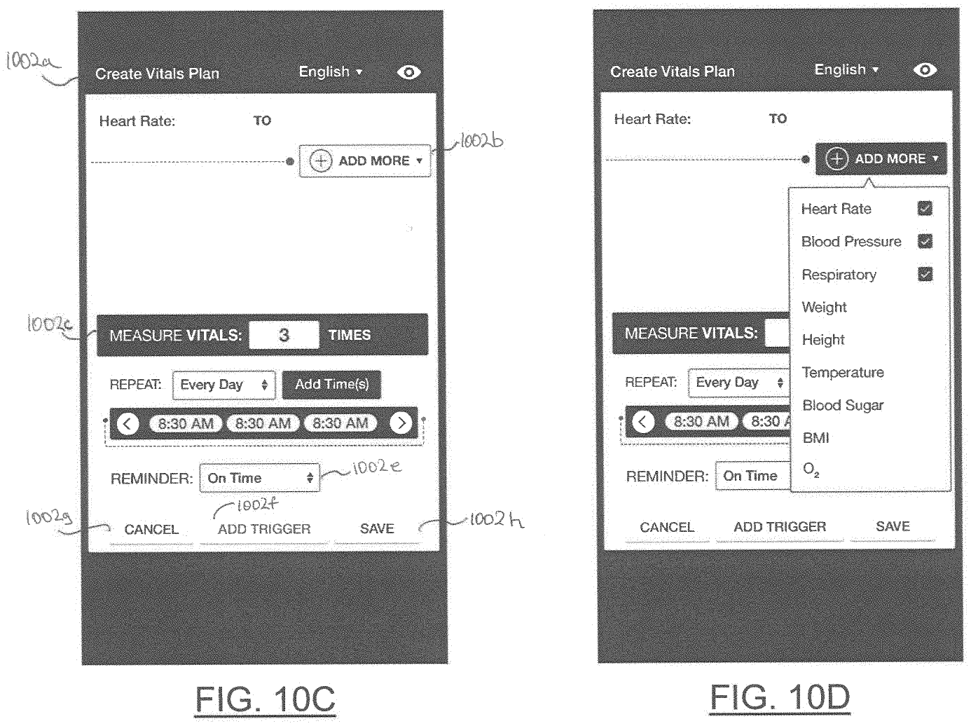

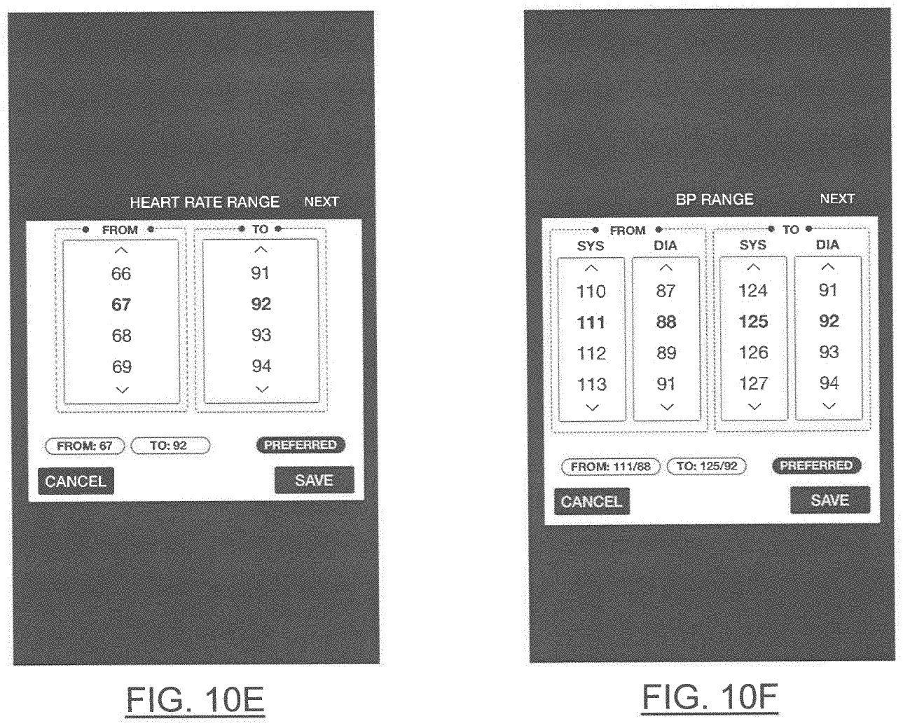

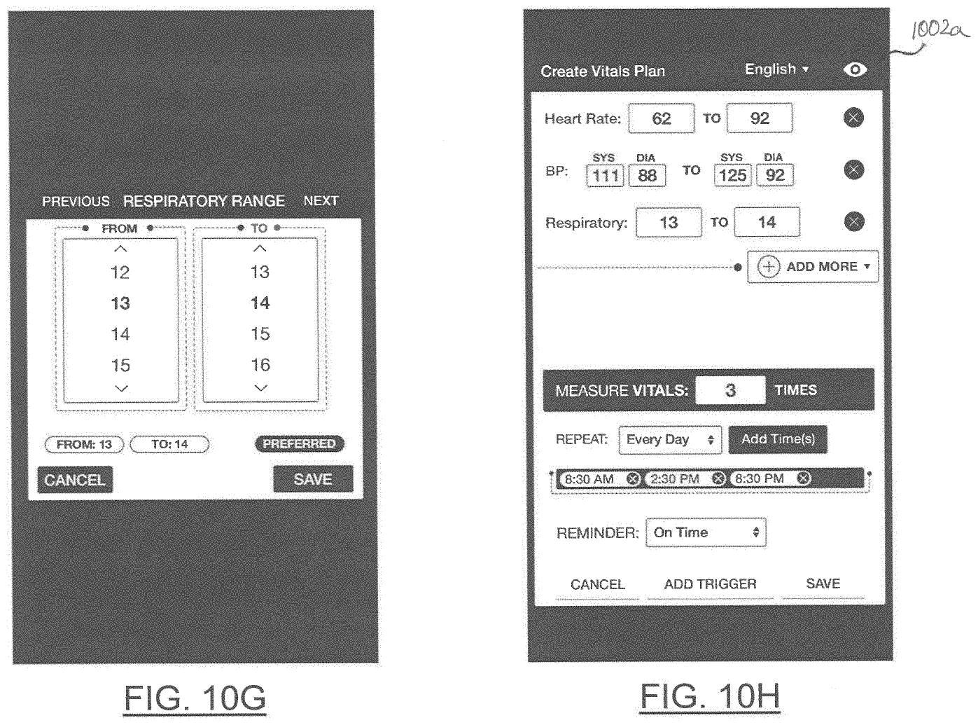

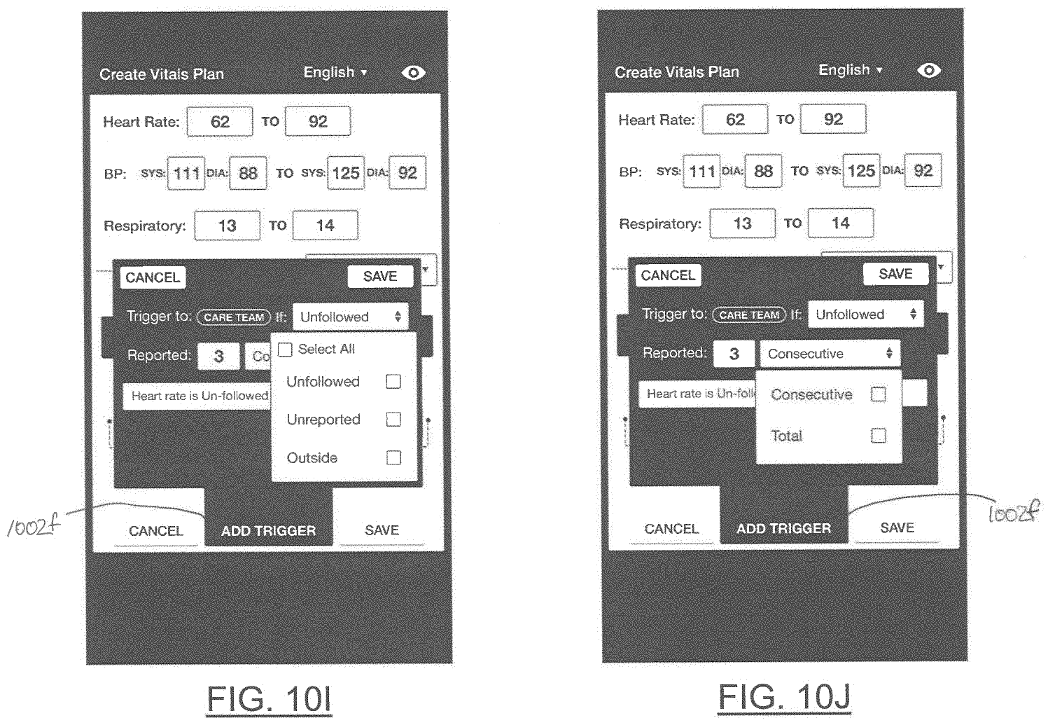

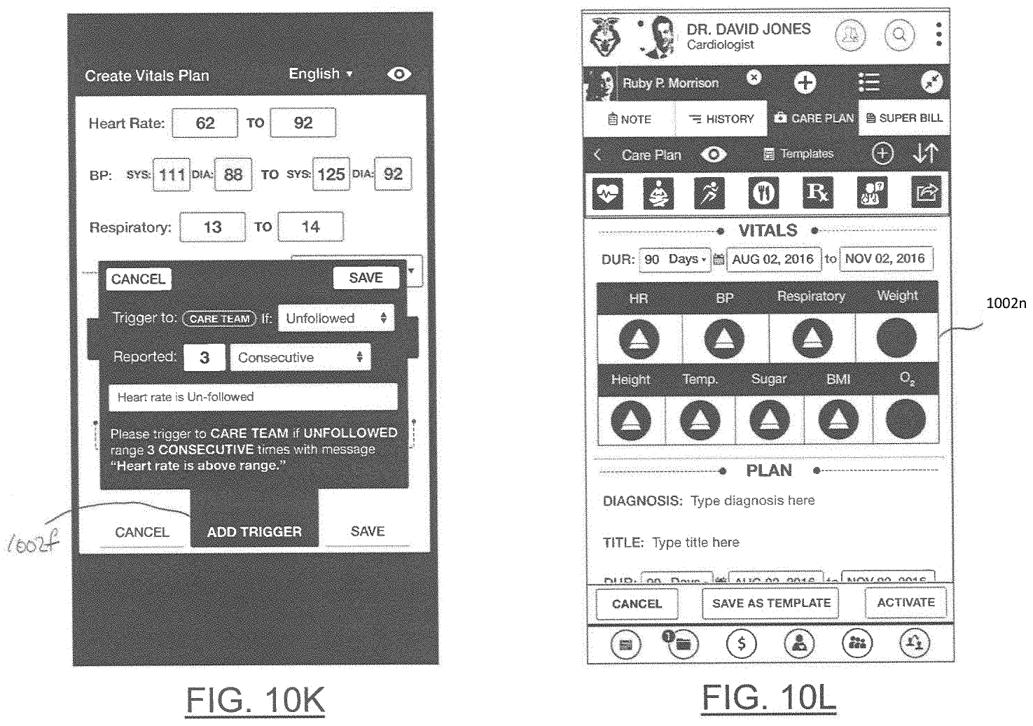

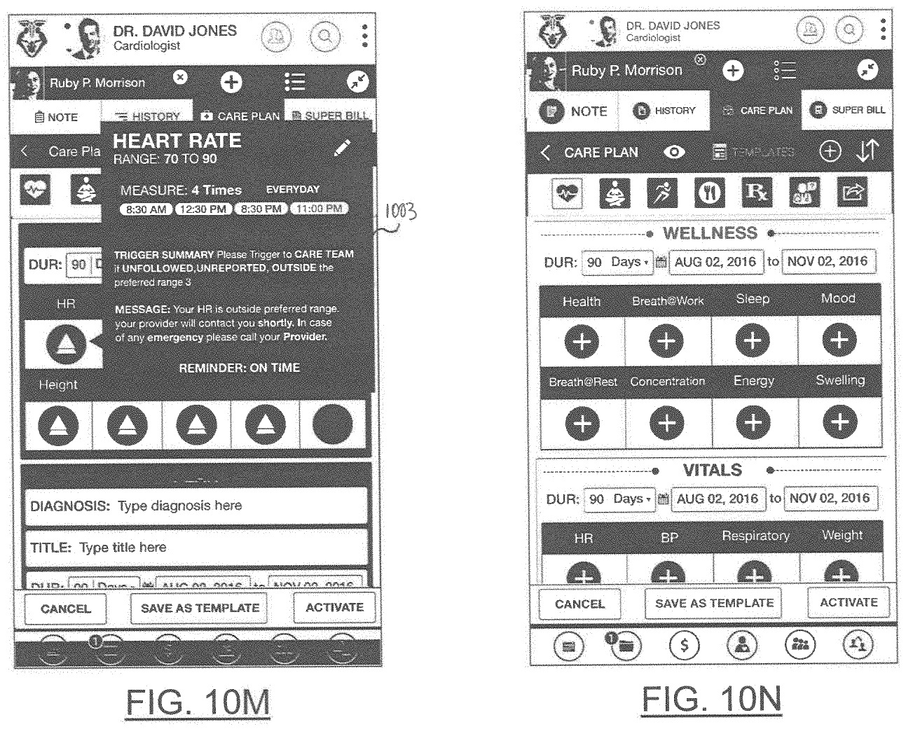

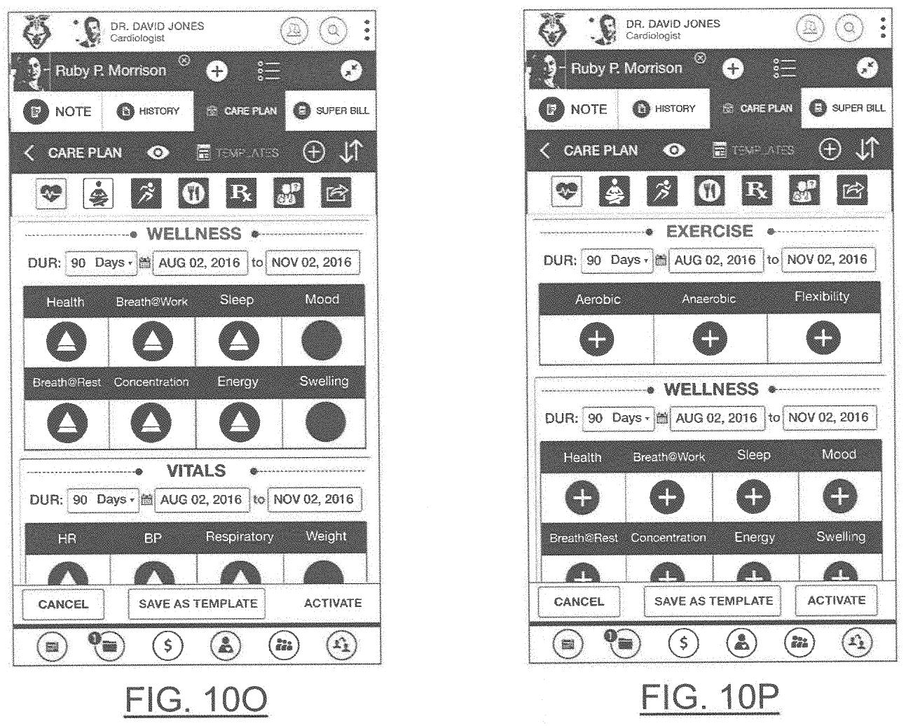

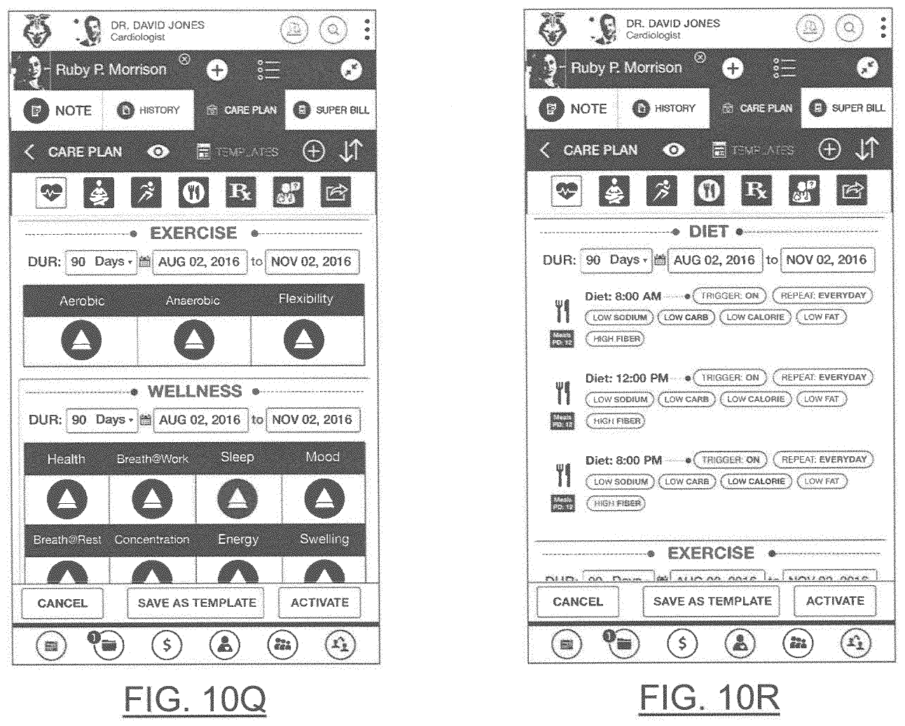

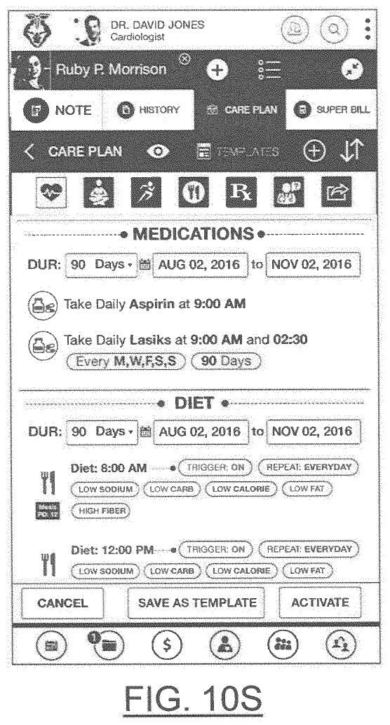

During or after the meeting with the patient, the care provider, using the patient monitoring platform 101, initiates the process of generating a care plan for the patient to follow based on the care provider's diagnosis of the patient. Additional details of the generation of the care plan are provided herein in the context of FIG. 10A and FIG. 10B. The care provider, using the patient monitoring platform 101, transmits the care plan to the patient via the patient monitoring platform 101, which displays an alert or a notification to the patient. In some embodiments, the alert or notification is displayed in the GUI 200c, described above in the context of FIG. 2C. The patient monitoring platform displays care plans received by a user, such as the patient, from care providers in response to the selection of the graphical item 202i. An example of a GUI displaying care plans received by a user is shown in FIG. 7A.

Turning now to FIG. 7A, there is shown an application screen 700e displaying GUI 700f. The patient monitoring platform 101 displays the GUI 700f in response to the selection of the graphical item 202i. The GUI 700f includes multiple graphical items, including graphical items 711a, 711b, 711c, 711d, and each graphical item is associated with different care plans for the patient from different care providers. Each of the graphical items 711a, 711b, 711c, 711d, includes additional graphical items that allow a user to interact with and the patient monitoring platform 101 displays additional information associated with the care plan as shown with graphical item 711a in FIG. 7A. The patient monitoring platform 101 displays a status for each care plan, such as “INACTIVE,” “ACTIVE,” etc., in a graphical item, such as graphical item 701a, and in response to the selection of the graphical item 701a, the patient monitoring platform 101 prompts the patient to select whether the patient desires to activate or deactivate the care plan. The graphical item 701a displays such a prompt and includes the graphical items 701b, 701c, as shown in FIG. 7B. In response to the selection of the graphical item 701c, the patient monitoring platform 101 transmits a message to the care provider indicating that care plan has been deactivated by the patient if the care plan was previously activated. In some embodiments, in response to the selection of the graphical item 701c, the patient monitoring platform 101 deletes the received care plan only after a threshold amount of time has elapsed since the patient received the care plan. In some embodiments, in response to the selection of the graphical item 701c, the patient monitoring platform 101 displays a graphical item (not shown) specifying that the care plan has been rejected.

In response to the selection of the graphical item 701b, the patient monitoring platform 101 activates the care plan and changes the status in the graphical item 701a to indicate the current status of the care plan, as shown in FIG. 7C. Once the care plan is activated, the patient monitoring platform 101 automatically populates the calendar application associated with the patient's profile with tasks and reminders that the patient must complete to sufficiently follow the activated care plan. An example of a calendar application of the patient automatically populated with the tasks and reminders of the care plan is displayed in GUI 703a, presented in the application screen 700b, as shown in FIG. 7D. The application screen 700b also presents navigational graphical items 202, 204, GUI 700f, in split screen. The GUI 703a is also associated with the navigation graphical item 204a and the patient monitoring platform 101 displays the GUI 703a in response to the selection of the navigational graphical items 204a.

The GUI 703a displays the calendar application associated with the patient profile including the tasks and reminders auto-populated from the care plan activated by the patient. The GUI 703a includes graphical items 703b, 703c, 703d, 703e. The graphical item 703b is associated with a task from care plan and displays the time at which the task should be performed by the patient and a description of the task to perform. In some embodiments, the patient monitoring platform 101 displays additional information about the task in response to the selection of the graphical item 703b. The patient monitoring platform 101 allows the patient to search for a particular task or reminder by entering search terms in the graphical item 703c and the patient monitoring platform 101 searches for the task or reminder in response to receiving the search terms. The patient monitoring platform 101 initiates the process of adding a task to the calendar application associated with the patient profile in response to the selection of the graphical item 703d, which is associated with the process of adding a task. The graphical item 703e is associated with different views of the calendar application and the patient monitoring platform 101 displays a view of the calendar application based on selected portion of the graphical item 703e. An enlarged view of the GUI 703a is shown in FIG. 7E. In the enlarged view, GUI 703a includes navigational graphical items 202, 204.

Each task from the care plan is associated with a beginning trigger time and the patient monitoring platform 101 displays an alert once the beginning trigger time of the application is reached. An example of the alert is graphical item 704a, shown in FIG. 7F. The graphical item 704a is an alert associated with the task displayed by the graphical item 703b (shown in FIG. 7D). The task displayed by the graphical item 703b is to provide vital data of the patient to the patient monitoring platform 101. The graphical item 704a includes graphical item 704b, 704c, 704d, 704e, 704f. The patient monitoring platform 101 suspends the alert for a threshold amount of time and closes the graphical item 704a, in response to the selection of the graphical item 704c. After the threshold amount of time has elapsed, the patient monitoring platform resumes the suspended alert and displays the graphical item 704a again. The patient monitoring platform 101 displays a graphical item configured to receive text in response to the selection of the graphical item 704e to allow a user to provide inputs regarding the task he/she is performing. The patient monitoring platform 101 closes the graphical item 704a in response to the selection of the graphical item 704f.

The graphical item 704b is associated with one of the vitals of the patient, for example, the heart rate of the patient. In response to the selection of the graphical item 704b, the patient monitoring platform 101 initiates the process of receiving data related to the heart rate of the patient. In some embodiments, the patient monitoring platform 101 displays graphical item 705a, as shown in FIG. 7G, in response to the selection of the graphical item 704b. The graphical item 705a includes graphical items 705b and 705c. The graphical item 705b is configured to display a wide range of numerical values and the patient may scroll through the range of numerical values by selecting the up or down arrows displayed in the graphical item 705b. The patient may provide his/her heart rate to the patient monitoring platform 101 manually by selecting one of numerical values. In some embodiments, the patient monitoring platform 101 displays a graphical item (not shown) requesting a confirmation to transmit the selected numerical value to the patient monitoring platform 101 as the heart rate of the patient. In some embodiments, where the patient monitoring platform 101 is being accessed from a computing device equipped with a heart rate sensor, the patient monitoring platform receives the heart rate of the patient in response to the patient placing his/her finger on the graphical item 706, included in the graphical item 705b. The graphical item 705a includes graphical item 705d to allow users to input additional information regarding the vitals data being input to the patient monitoring platform 101, graphical item 705e to cancel the process of entering the vitals data, and graphical item 705f to save the entered vitals data, An example of a user successfully entering his/her vitals data, such as heart rate data, into the patient monitoring platform 101 is shown in FIG. 7H. Similarly, the patient monitoring platform 101 presents one or more graphical items to receive data related to other vitals of the patient. An example of successfully providing all vitals required by the activated care plan is displayed by the graphical item 704a, as shown in FIG. 7I. In some embodiments, the patient monitoring platform 101 transmits the vitals data to the care provider in response to the selection of the graphical item 704d.

The patient monitoring platform 101 provides a care provider options to select secondary alerts for each task specified in the care plan. Examples of secondary alerts include, but are not limited to, interactive email response, interactive voice response, interactive text response to the patient. As described herein, interactive voice response is a telephone call to the patient that requests the patient to press or dial or provide any input indicating whether or not the patient has completed the task. As described herein, interactive email response is an email sent to the patient that presents the patient with two options, one to confirm that the patient has completed the task and the second to confirm that the patient has still not completed the task, and requests the users respond back with one of the two options. As described herein, interactive text response, similar the interactive email response, presents the patient with two options, one to confirm that the patient has completed the task and another to indicate that the patient has not completed the task.

The patient monitoring platform 101 is configured to use one of the secondary alerts for a task, if a task is not completed by the end trigger time associated with the task. For example, if the patient was required to provide data related to the patient successfully taking a dosage of medication to the patient monitoring platform 101 by 10:00 am and the end trigger time for the task is 10:15 am and, if the secondary alert is an interactive voice response, the patient monitoring platform may initiate a telephone call with the patent if the time is past 10:15 am, and based on the input from the patient, the patient monitoring platform 101 may update the status of task to complete or leave it as incomplete. In some embodiments, the care provider selects multiple secondary alerts for a single task, and in response, the patient monitoring platform 101 requests the care provider to order or rank the secondary alerts and the patient monitoring platform 101 uses the secondary alerts in order or rank of the selected secondary alerts. In some embodiments, the patient monitoring platform 101 cycles through the secondary alerts until the task is marked complete.

The care plan provided by a care provider may also include tasks for providing data related to diet of the user, such as the patient. Example GUIs and graphical items displayed by the patient monitoring platform 101 to allow users to provide the patient monitoring platform 101 with diet related data are shown in FIG. 7J, FIG. 7K, FIG. 7L, FIG. 7M. In some embodiments, the patient monitoring platform 101 presents graphical items to initiate image capturing in order to receive dietary data related of the patient. The care plan may also include tasks for providing fitness data to the patient monitoring platform 101, and example GUIs and graphical items displayed by the patient monitoring platform 101 to allow users to provide fitness data to the patient monitoring platform 101 are shown in FIG. 7N, FIG. 7O. In some embodiments, the patient monitoring platform 101 may automatically retrieve data related to certain tasks, such as entering exercise or fitness data, from the health monitoring and/or fitness tracking devices of the user that are operably coupled to the patient monitoring platform 101. The care plan may also include tasks to take medication at certain times and the patient monitoring platform 101 displays alerts for taking the medication at those times, as shown in FIG. 7P. In some embodiments, the patient monitoring platform 101 also displays an alert at a beginning time and an alert at an end time of a range of time within which the user is to consume the medication, as shown in FIG. 7P, FIG. 7Q, FIG. 7R.

The care plan may also include tasks related to providing the patient monitoring platform 101 data related to the patient's subjective views on how he feels, referred to herein as “wellness data.” The patient monitoring platform 101 displays graphical items that allow users to enter wellness data in multiple categories as shown in FIG. 7S. The patient monitoring platform 101 displays graphical items shown in FIG. 7T to facilitate an efficient input mechanism for users to provide data on how he/she feels. The graphical items shown in FIG. 7T lessen the burden on the users in identifying on how they feel while allowing them to provide their identified feelings in a format that can be efficiently processed by the patient monitoring platform 101. As shown in FIG. 7U, 7V, the patient monitoring platform 101 displays wellness data that the user successfully entered for the categories that were associated with the tasks related to providing wellness data.

The patient monitoring platform 101 displays graphical items to allow a user, such as the patient, to view details of the activated care plan. Graphical item 701d, shown in FIG. 7W is an example of such a graphical item. In response to the selection of the graphical item 701d, the patient monitoring platform 101 displays graphical item 701e. The graphical item 701e includes additional graphical items, displaying multiple options for viewing details of the care plan and, in response to the selection of one of the graphical items displaying an option, the patient monitoring platform 101 displays a GUI presenting the details of the care plan corresponding to the selected option or the option displayed by the selected graphical item. For example, in response to selection of the graphical item displaying the option Summary, the patient monitoring platform 101 displays a summary of the care plan, including summary of the tasks of the care plan in a GUI, such as GUI 700h, shown in FIG. 7X. In some embodiments, the patient monitoring platform 101 displays the GUI 700h in response to the selection of the graphical item 202i.

Turning now to FIG. 7X, there is shown application screen 700g displaying the GUI 700h. The GUI 700h includes graphical items that allow a user to select a different view of the care plan showing different details of the care plan. The GUI 700h also includes graphical items that allow a user to view progress that the user has made through a care plan. Examples of such graphical items are graphical items 712a, 712b. In response to the selection of the graphical item 712b, the patient monitoring platform 101 displays application screen 700c displaying the GUI 700d, as shown in FIG. 7Y. Turning now to FIG. 7Y, the GUI 700d includes navigational graphical items 202, 204, graphical items 707a, 707b, 707c, 707d, 707e, 707f, 707g, collectively graphical items 707; and graphical items 712a, 712b. In some embodiments, in response to the selection of the navigational graphical item 202i, the patient monitoring platform 101 displays GUI 700d. The patient monitoring platform 101 displays information, including contact information, of the care provider associated with the care plan in response to the selection of the graphical item 707f. In some embodiments, in response to the selection of the graphical item 707f, the patient monitoring platform 101 contacts the care provider 101 using the communication information of the care provider associated with the care plan. The graphical item 707g is configured to receive input for exporting data associated with the care plan from the patient monitoring platform 101 and in response to the selection of the graphical item 707g, the patient monitoring platform 101 exports the data associated with the care plan. In some embodiments, the patient monitoring platform 101 displays graphical items (not shown) to allow a user to select a particular subset of data associated with the care plan to export to the user. In some embodiments, the patient monitoring platform 101 is configured to display information related to the all of the care plans associated with the patient. In some embodiments, if a care plan was rejected or canceled by the patient, the patient monitoring platform 101 may display a graphical item indicating the status of the care plan.

Each of the graphical items 707 is associated with the progress of the user, such as the patient, in a particular section of care plan and selection of one of the graphical items 707 causes the patient monitoring platform 101 to display data corresponding to the section of the care plan over a period of time. For example, the graphical item 707a is associated with the progress of the vitals of the patient and in response to the selection of the graphical item 707a, the patient monitoring platform 101 displays data related to the vitals of the patient over a period of item, as shown in FIG. 7Y. In some embodiments, as shown in FIG. 7Y, the patient monitoring platform 101 displays one or more graphical items (not shown) displaying data over a period of time for each of the vitals of the patient. Similarly, the graphical item 707b is associated with progress of the wellness data of the patient, the graphical item 707c is associated with the progress of the exercise data of the patient, the graphical item 707d is associated with the progress of the diet data of the patient and the graphical item 707e is associated with the progress of the medication data of the patient. In response to the selection of the Selection of graphical items 707b, 707c, 707d, 707e, the patient monitoring platform 101 displays corresponding data.

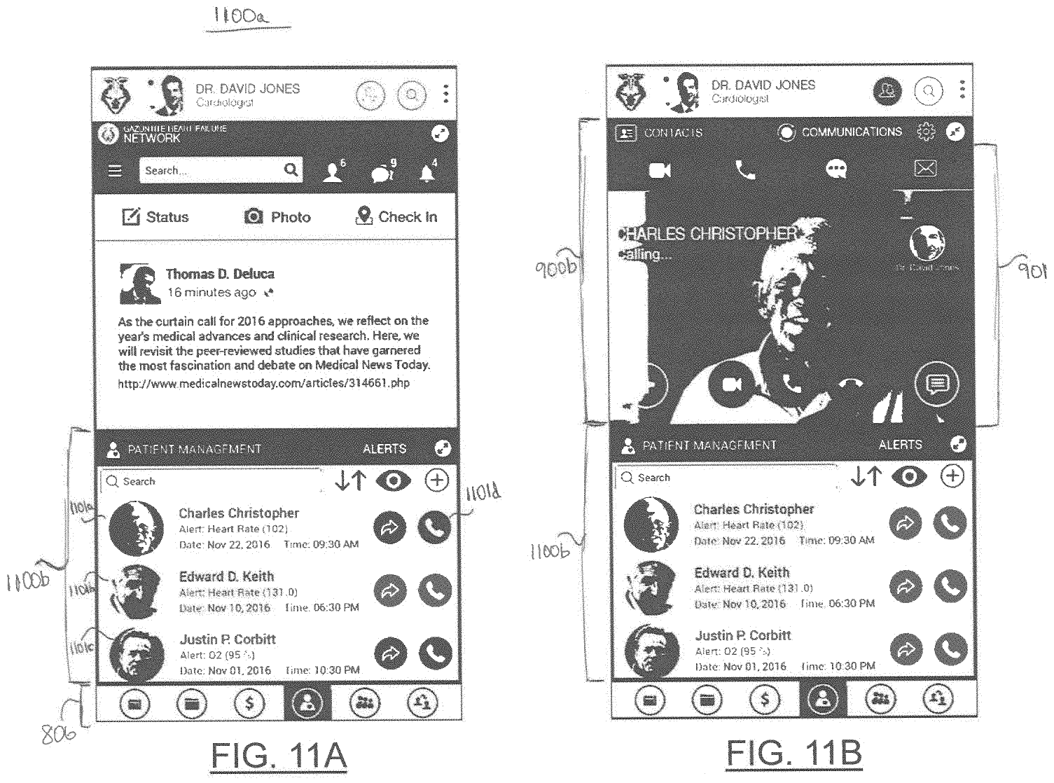

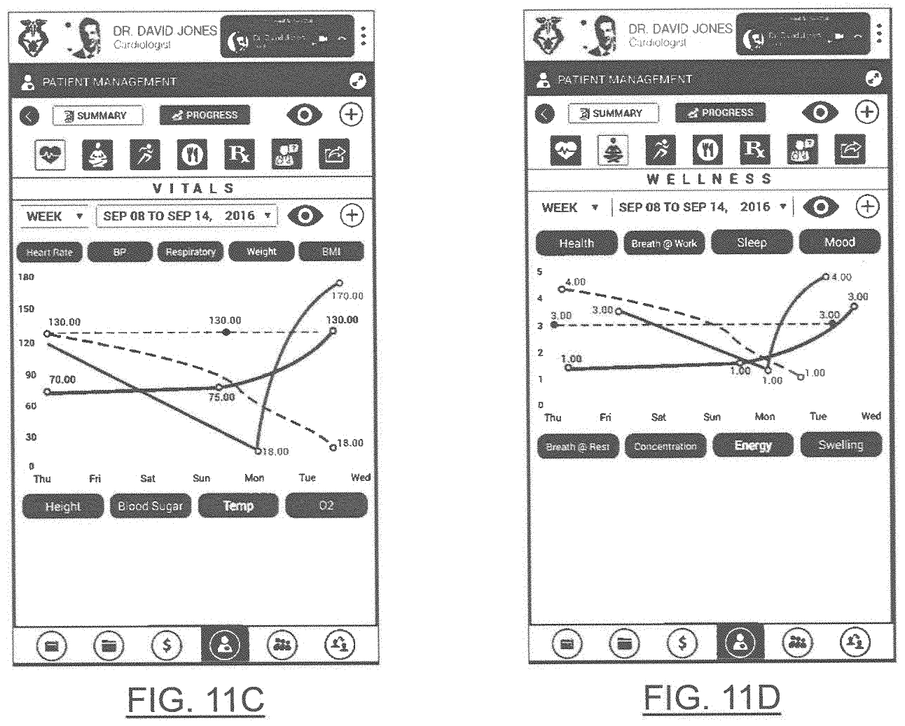

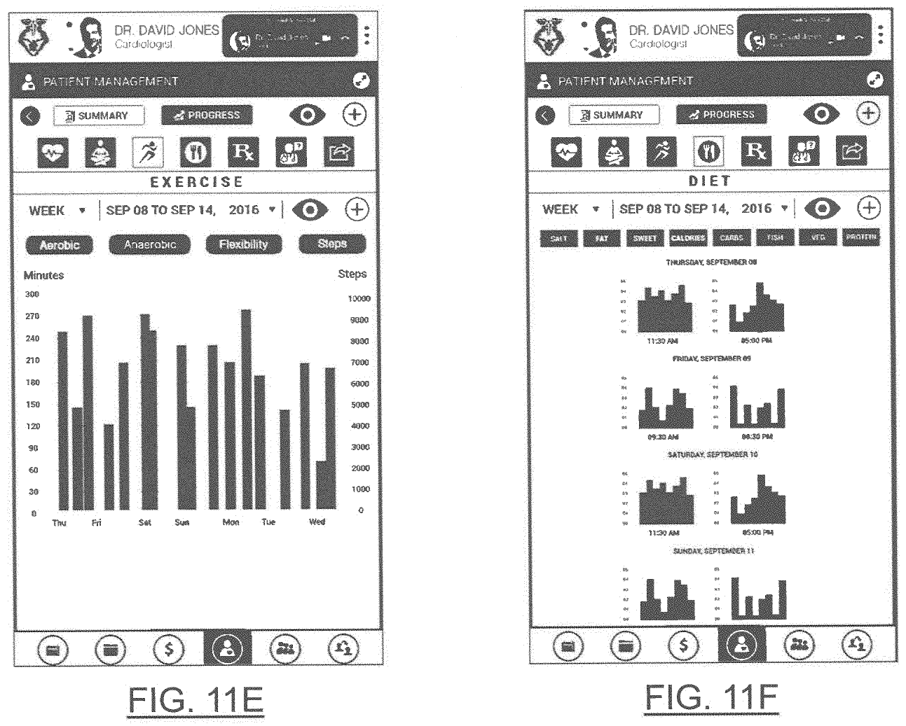

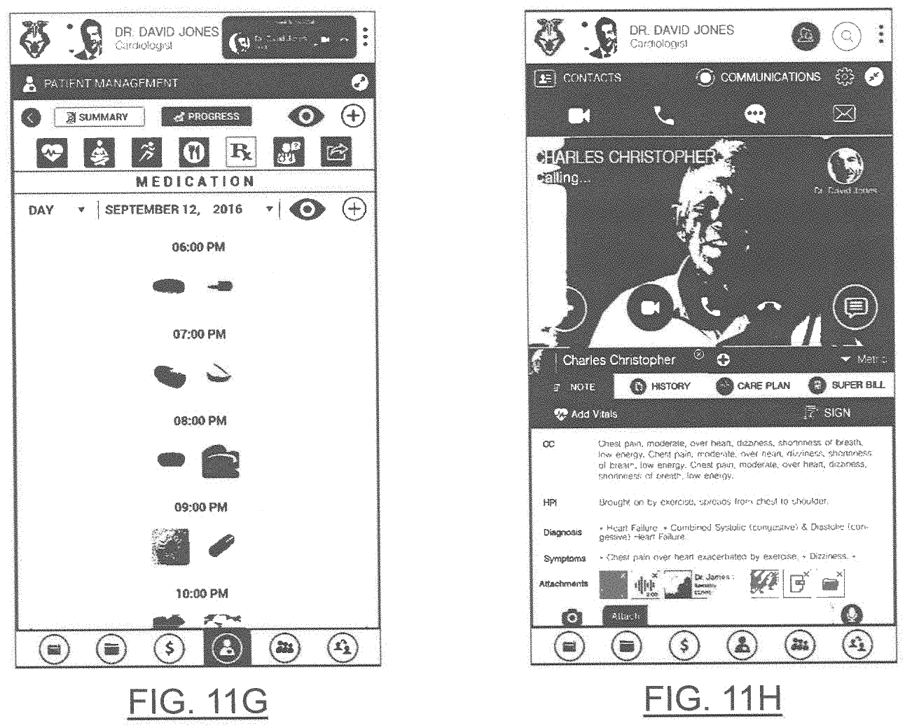

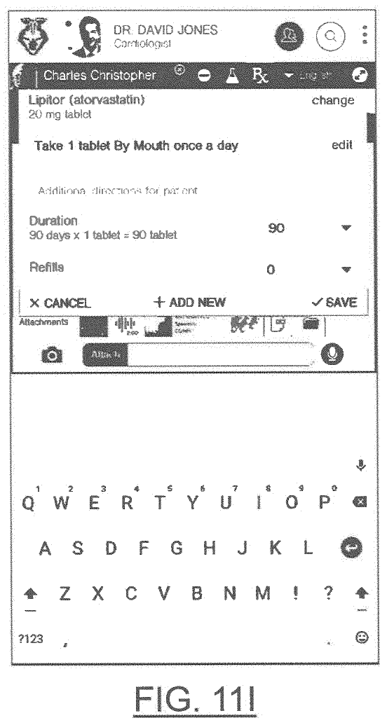

The patient monitoring platform 101 is configured with a set of rules to determine whether or not certain data related to the care plan and provided to the patient monitoring platform 101 is outside the threshold parameters for that type of data. For example, the patient monitoring platform 101 determines whether the heart rate data of the patient received by the patient monitoring platform 101 is outside the threshold parameters by comparing the heart rate of data received by the patient monitoring platform 101 and the set of rules that specify heart rates that are high end and low end of a healthy heart rate. If the patient monitoring platform 101 determines that the heart rate of the patient is not a healthy heart rate, then the patient monitoring platform 101 generates and transmits an alert to the care provider warning the care the provider that the patient's heart rate is not at healthy level. An example of an alert regarding a patient, displayed to a care provider is shown in FIG. 11A. The patient monitoring platform 101 provides the care provider with multiple options to respond to the warning including, but not limited to, emailing the patient, sending a text message to the patient, calling a patient.

The patient monitoring platform 101 is configured to make and receive telephone calls and in response to receiving a call, the patient monitoring platform 101 displays a graphical item indicating that the patient is receiving a call. The graphical item 709a, shown in FIG. Z, is an example of such a graphical item. The graphical item 709a displays to the users graphical items 709b, 709c in order to provide the user options to accept or reject the incoming call. In response to the selection of the graphical item 709c, the patient monitoring platform 101 terminates the incoming call. The patient monitoring platform 101 accepts the incoming call in response to the selection of the graphical item 709b. If the incoming call is a video call, then in response to the selection of the graphical item 709b, the patient monitoring platform 101 initiates a video call with the caller and displays the video of the caller in a GUI, such as GUI 700j, shown in FIG. 7Z1. Turning now to FIG. 7Z1, there is shown an application screen 700i displaying the GUI 700j and the GUI 700d in split screen. The GUI 700j displays the video of the caller, the video of the user, and graphical items configured to receive input for other modes of communication, including email, instant message, etc. The GUI 700j displays graphical items configured to receive input to switch from the video call to an audio call and displays graphical items configured to receive input to end the call. In response to the selection of these graphical items, the patient monitoring platform 101 performs corresponding actions.

The patient monitoring platform 101 allows the user to navigate through different GUIs, applications, sub-applications, and/or functionalities of the patient monitoring platform 101 while the user is communicating with the caller, the care provider, via video call or audio call, as shown in FIG. 7Z2. In FIG. 7Z2, the user resumes GUI 700d into full screen from the split screen and the user is able to view progress data associated with the care plan while communicating with the care provider. The patient monitoring platform 101 displays GUI 700d in full screen in response to a selection of any portion of the display of the computing device displaying GUI 700d. The patient monitoring platform 101 displays graphical item 709d in response to the selection of any portion of the display of the computing device displaying GUI 700d. The graphical item 709d is configured to receive input to display the GUI 700j and in response to selection of the graphical item 709d, the patient monitoring platform 101 displays the GUI 700j. As shown in FIGS. 7Z2, 7Z3, 7Z4, 7Z5, 7Z6, 7Z7, 7Z8, the user, such as the patient, may view different data, such as data related to vitals, exercise, wellness, diet, and/or medication of the user, associated with the care plan, In some embodiments, the patient monitoring platform 101 displays the GUI 700j in split screen with the GUI that is displayed in full screen, as shown in FIG. 7Z8.

Based on the telephone conversation, the care provider may modify the care plan transmitted to the patient and the patient monitoring platform 101 automatically synchronizes the changes made by the care provider to the care plan associated with the patient's profile. Additionally, in response to the modification of the patient's care plan, the patient monitoring platform 101 automatically updates the entries made to the calendar application, if necessary. The patient monitoring platform 101 is configured to automatically record a voice or video conversation either made or received using the patient monitoring platform 101 and one or more metrics of the conversation, such as start time, end time, date, duration, etc. After a video or voice call that is initiated is terminated, the patient monitoring platform 101 displays a graphical item that requests the users involved in the telephone conversation to save the recording of their conversation. An example of such a graphical item displayed to the user who is non-care provider, such as a patient, is graphical item 710a, shown in FIG. 7Z9. The patient monitoring platform 101 presents a graphical item similar to the graphical item 710a to the care provider, involved in the video or voice call. The graphical item 710a includes graphical items 710b, 710c, 710d, 710e, and displays one or metrics about the call, such as the name of the other user in the conversation, the duration of the call, etc. The graphical items 710b, 710c are configured to receive text from the user related to the title of the recording and the search tags with which to associate the recording.

While the patient monitoring platform 101 automatically records the video or voice conversation, the patient monitoring platform 101 only allows a user to save the recording if both users, the patient and the care provider, agree that the recording should be saved. Therefore, in response to the selection of the graphical item 710d by a first user, such as the patient, the patient monitoring platform 101 closes the graphical item 710a and transmits a notification to the second user, such as the care provider, indicating that the first user does not wish to save the call and prevents the second user from saving the file. If a first user, such as the patient, selects the graphical item 710e, then the patient monitoring platform 101 transmits a notification to the second user, such as the care provider, indicating that the first user desires to save the video or voice conversation recording and presents a graphical item (not shown) that allows the second user to grant the permission to save the recording or instructs the patient monitoring platform 101 to prevent the saving of the recording.

If both users involved in the conversation agree to saving the recording, then the patient monitoring platform 101 saves the recording in a data storage unit operably coupled to the patient monitoring platform 101 and displays a graphical item (not shown) in a GUI (not shown), where the graphical item is associated with process of playing the saved recording and the patient monitoring platform 101 plays the saved recording in response to the selection of the graphical item. For care providers, the patient monitoring platform 101 displays a graphical item that requests the care provider whether he/she wishes to generate and transmit a bill to the health insurance provider in a message that includes the saved recording as evidence of the service provided to the patient remotely by the care provider. The patient monitoring platform 101 associates the recorded video or voice conversation as part of the health-related files of the user and displays a graphical item, such as graphical item 713 (shown in FIG. 7Z10), in the GUI that the patient monitoring platform 101 displays in response to the selection of the navigational graphical item 202f Turning now to FIG. 7Z10, the graphical item 713 is configured to receive input to play the recorded video or voice conversation, and in response to the selection of the graphical item 713, the patient monitoring platform 101 plays the recorded conversation. In some embodiments, the patient monitoring platform 101 displays additional information associated with the recorded conversation and graphical items configured to receive input to play the recorded conversation in response to the selection of the graphical item 713. The patient monitoring platform 101 allows users to view their recent conversations in response to the selection of the navigational graphical item 202a, as shown in FIG. 7Z11.

Although described in the context of a user being a patient, the patient monitoring platform 101 provides a user interface for a care provider as well. Referring to FIG. 2B, GUI 200b is an example user interface displaying a login screen to a care provider, such as a doctor, etc. GUI 200b includes graphical items 201e, 201f, 201g, 201h. Similar to graphical items 201a and 201b, the graphical items 201e and 201f are configured to receive a username of the care provider and a password associated with the username, respectively. The graphical items 201g and 201h are similarly configured as the graphical items 201c and 201d, and, upon selection of the graphical items 201g or 201h, the patient monitoring platform 101 determines whether the username and password are accurate and associated with a care provider registered with the patient monitoring platform 101, or register a care provider with the patient monitoring platform 101, respectively.

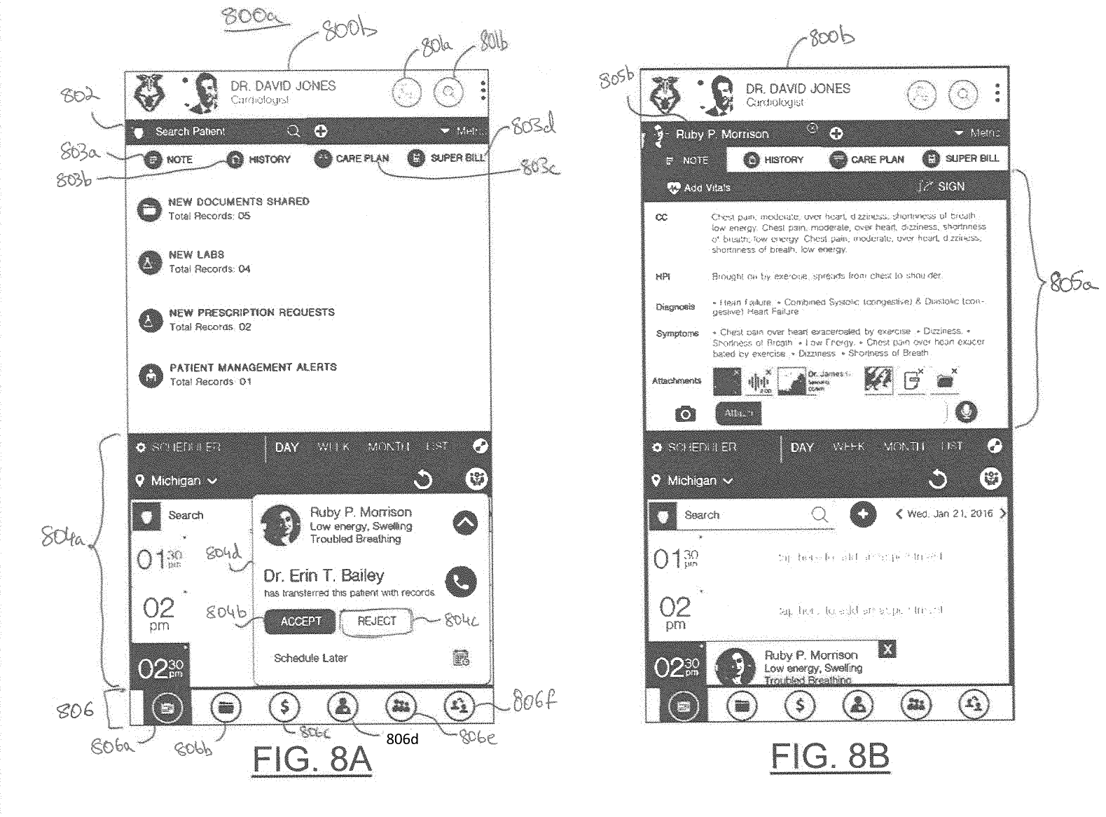

Turning now to FIG. 8A, there is shown an application screen 800a displaying GUI 800b. The GUI 800b displays an example GUI for a care provider that successfully logs into the patient monitoring platform 101. The GUI 800b includes navigational graphical items 801a, 801b, 803a, 803b, 803c, 803d, graphical items 802, 804a, 804b, 804c, 804d, navigational graphical items 806a, 806b, 806c, 806d, 806e, 806f, collectively navigational graphical items 806. The graphical item 801a is associated with similar functionality of the patient monitoring platform 101 as the graphical item 202a and in response to the selection of the graphical item 801a, the patient monitoring platform 101 displays the users of the patient monitoring platform 101 that are part of the network of the care provider.

The graphical item 801b is associated with the same functionality of the patient monitoring platform 101 as the graphical item 202b and, as described above, in response to the selection of the graphical item 202c, the patient monitoring platform 101 displays the user interface for the search functionality associated with the patient monitoring platform 101. The graphical item 802 is configured to receive text and the patient monitoring platform 101 searches for a patient matching the text received by the patient monitoring platform 101.

The navigational graphical item 803a is associated with notes entered into patient monitoring platform 101 for a particular patient by the care provider and in response to the selection of the navigational graphical item 803a, the patient monitoring platform 101 displays the notes associated with the patient for whom the care provider searched. Graphical item 805a, shown in FIG. 8B, is an example of the patient monitoring platform 101 displaying the notes entered by the care provider for the patient displayed in the graphical item 805b. The patient monitoring platform 101 includes all files transmitted to the care provider from the patient as part of the notes the care provider entered into the patient monitoring platform 101, as displayed in the graphical item 805b.

Turning back to FIG. 8A, the patient monitoring platform 101 displays the history of visits between a patient and a care provider in response to the selection of the navigation graphical item 803b. The patient monitoring platform 101 displays one or more care plans created or saved by the care provider in response to the selection of the graphical item 803c. Additionally, the patient monitoring platform 101 displays a graphical item (not shown) associated with initiation of the process of creating a care plan and the patient monitoring platform 101 initiates the process of creating the care plan in response to the selection of the graphical item. The patient monitoring platform 101, in response to the selection of the graphical item 803d, displays one or more bills for patients generated by the care provider. In some embodiments, the patient monitoring platform 101 displays graphical items (not shown) that cause the patient monitoring platform 101 to initiate the process of generating a bill to the patient or the patient's health insurance provider.

The graphical item 804a displays a calendar application associated with the care provider's profile in the patient monitoring platform 101 and includes graphical items 804b, 804c, 804d. The graphical item 804a is associated with the similar functionality as the GUI 703a in FIG. 7D. As described above, the patient monitoring platform 101 is operably coupled with medical facility administrative systems including systems that schedule appointments with the care provider. The patient monitoring platform 101 automatically populates the calendar entries within the calendar application associated with the care provider using the care provider's scheduling data from the operably coupled medical facility administrative systems. The graphical item 804d displays a request from a patient for an appointment with the care provider. The graphical item 804d includes the graphical items 804b, 804c. In response to the selection of the graphical item 804c, the patient monitoring platform 101 removes the graphical item 804d from being displayed by the patient monitoring platform. In response to the selection of the graphical item 804b, the patient monitoring platform 101 saves the appointment in a data storage unit and displays a link to the appointment in the calendar application. Additionally, the patient monitoring platform 101 removes the times included in the accepted appointment in order to display time periods at which the care provider is not available.

The graphical item 806a is configured to receive input to display the calendar application associated with the care provider's profile and in response to the selection of the graphical item 806a, the patient monitoring platform 101 displays the associated calendar application in a graphical item, such as the graphical item 804a. The graphical item 806b is configured to receive input to display folders associated with the care provider's profile and in response to the selection of the graphical item 806b, the patient monitoring platform 101 displays the folders associated with the care provider's profile in a GUI (not shown) including a graphical item (not shown). The graphical item 806c is configured to receive an input to display transactional information of the care provider including, but not limited to, summary of revenues and expenses, bills for the patients that are unpaid, and bills for the patients that are paid, and in response to the selection of the graphical item 806c, the patient monitoring platform 101 displays corresponding information in a GUI (not shown).

The graphical item 806d is configured to receive input to display alerts from patients of the care provider or alerts related to the patients of the care provider, and in response to the selection of the graphical item 806d, the patient monitoring platform 101 displays a GUI displaying the alerts, as shown in FIG. 11A. The graphical item 806e is configured to receive an input to display users that are part of the care provider's network and in response to selection of the graphical item 806e, the patient monitoring platform displays a GUI presenting users registered with the patient monitoring platform 101 that are associated with the profile of the care provider as part of the care provider's network. The graphical item 806f is configured to receive input to display contacts of the care provider and, in response to the selection of the graphical item 806f, the patient monitoring platform 101 displays a GUI (not shown) displaying contacts of the care provider and multiple graphical items (not shown) per contact, each configured to receive input for a mode of communication with the contacts. The patient monitoring platform 101 initiates a mode of communication with the contacts based on the selection of the graphical items configured to receive input for communication with the contacts.

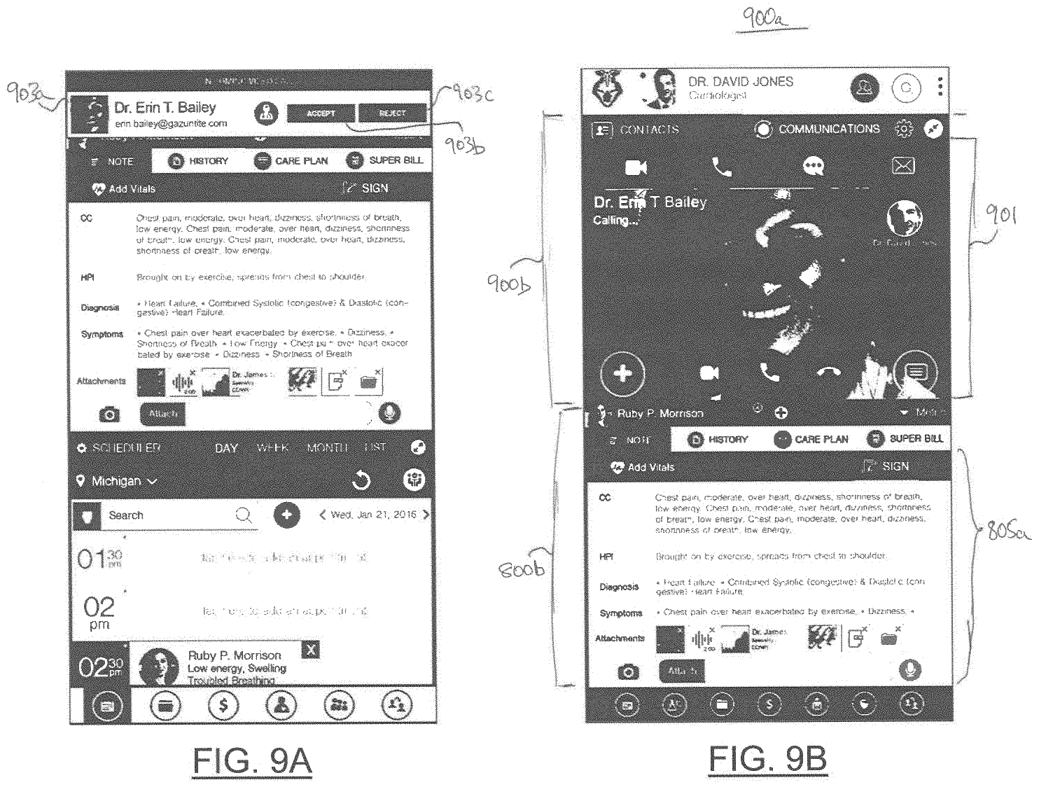

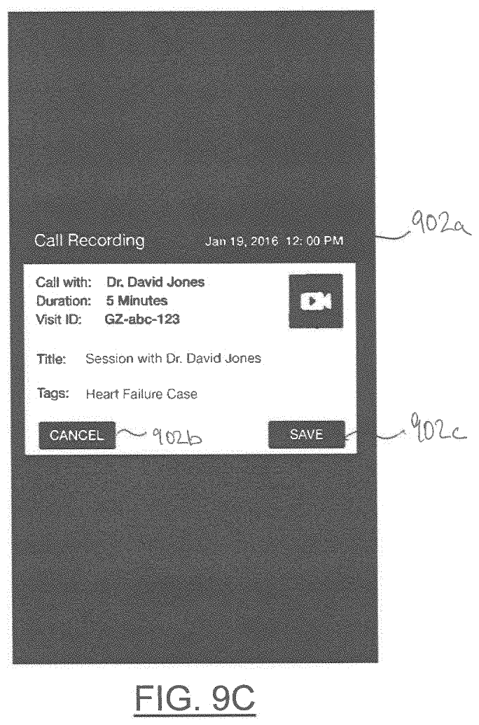

In response to the selection of the graphical item 801a or 806f, the patient monitoring platform 101 displays one or more graphical items (not shown) to receive input for searching for other care providers and displays contact details of the care providers in one or more graphical items. For example, if a patient sought the medical care from a first care provider, such as cardiologist, and is now visiting a second care provider, such as the patient's regular physician or family doctor, the second care provider may wish to consult with the first care provider regarding the care plan provided to the patient and using the patient monitoring platform 101, the second care provider may search for the first care provider and initiate a communication with the first care provider. If the second care provider communicates with the first care provider by calling the first care provider, then the first care provider will face challenges in being able generate a bill with sufficient evidence to prove that the first care provider spent time diagnosing and providing services to the patient now visiting the second care provider. The patient monitoring platform 101 resolves this issue as described below in the context of FIG. 9A, FIG. 9B and FIG. 9C.

Turning now to FIG. 9A, there is shown a care provider receiving call from another care provider displayed by the patient monitoring platform 101 in a graphical item 903a. The graphical item 903a includes graphical items 903b, 903c, and is configured similarly as the graphical item 709a, shown in FIG. 7Z. In response to the selection of the graphical item 903c, the patient monitoring platform 101 terminates the incoming call. The patient monitoring platform 101 accepts the call in response to the selection of the graphical item 903b. If the incoming call is a video call, then in response to the selection of the graphical item 903b, the patient monitoring platform 101 initiates a video call with the caller and displays the video of the caller, as shown in FIG. 9B. Turning now to FIG. 9B, there is shown application screen 900a displaying GUI 900b displaying a video conversation between two users, such as two care providers, and GUI 800b in split screen. The GUI 900b includes graphical item 901 and the graphical item 901 displays information associated with the other user, such as the care provider. After the termination of the call, as described above, the patient monitoring platform 101 displays graphical item 902a, as shown in FIG. 9C, to the care provider and another similarly configured graphical item (not shown) to the other care provider in the conversation to save the recording from the video conversation. The graphical item 902a is associated with the same functionality as the graphical item 710a described in the context of FIG. 7I, and includes the graphical items 902b, 902c. In response to selection of the graphical items 902b by one of the care providers, the patient monitoring platform 101 closes the graphical item 902a for that care provider and prevents the other care provider from saving the recording. In response to the selection of the graphical item 902c by one of the care providers, the patient monitoring platform 101 displays a graphical item (not shown) to the other care provider requesting approval to save the recording by the first care provider. If the approval is granted, then the patient monitoring platform 101 saves the recording for both the care providers and prompts both care providers if they wish to transmit a bill to the health insurance provider of the patient including the saved recording as evidence that the two care providers rendered services to the patient even though at least one of the care providers is remote to the location of the patient.

Turning now to FIG. 10A, there is shown application screen 1000a presenting GUI 1000b for creation of a care plan by the care provider. The GUI 1000b includes the navigational graphical items 801a, 801b, 803a, 803b, 803c, 803d, graphical item 805b, graphical item 1001a. The patient monitoring platform 101 displays the patient for whom the care plan is being created via graphical item 805. The graphical item 1001a includes graphical items 1001b, 1001c, 1001d, 1001e, 1001f, 1001g, 1001h, 1001i, 1001j, 1001k, 1001l, 1001m. The graphical items 1001b, 1001c, 1001d, 1001e, 1001f are associated with vitals, wellness, exercise, diet, and medication, respectively. In some embodiments the patient monitoring platform 101 provides visual indication of the number of tasks selected within their associated portion of the care plan via graphical items (not shown).

The graphical item 1001g is configured to receive text from the care provider and the patient monitoring platform 101 uses the text to create a title for the care plan when the care plan is saved. The graphical item 1001h is configured to receive a date from the care provider, and in response to the receiving a date, the patient monitoring platform 101 associates the received date as the start date of the care plan. The patient monitoring platform 101 calculates the end date or the duration of the care plan based on the number of days entered into the graphical item 1001i and associates the end date and duration of the care plan as the calculated end date or duration of the care plan. The care provider specifies one or more secondary alerts for the care plan using the graphical item 1001j and the patient monitoring platform 101 initiates communication with the patient using the one or more secondary alerts specified in the graphical item 1001j.

In addition to transmitting the care plan to only the patient, the patient monitoring platform 101 allows the care provider to include one or more persons from the patient's network or care team. Using the graphical item 1001k, the care provider may select or remove persons that should receive the patient's care plan. In some embodiments, the patient monitoring platform 101 identifies the persons from the network or support team of the patient and displays the persons in a drop down box, thereby allowing the care provider to select the persons from the drop down list. In some embodiments, the patient monitoring transmits alerts to the care team in response to one or more secondary alerts being sent to the patient. The graphical item 1001l is configured to receive input to allow the care provider to provide to the patient monitoring platform 101 information related to communication with the care provider including, but not limited to, modes of communication for a patient to contact the care provider, contact details for each mode, etc. In response to the selection of the graphical item 1001l, the patient monitoring platform 101 displays GUIs and graphical items to allow the care provider to provide the corresponding information. The graphical item 1001m is configured to receive input to allow the care provider to transmit the care plan to the patient and in response to the selection of the graphical item 1001m, the patient monitoring platform 101 transmits the care plan to the patient. In some embodiments, the patient monitoring platform 101 displays additional graphical items to allow a care provider to export the care plan from the patient monitoring platform 101 to an external application, device, or system.

The care provider selects one or more tasks associated with vitals, wellness, exercise, diet, medications by selecting graphical items 1001b, 1001c, 1001d, 1001e, 1001e, respectively. For example, in response to the selection of the graphical item 1001b, the patient monitoring platform 101 displays graphical item 1001n, shown in FIG. 10B. The graphical item 1001n includes additional graphical items for each of vitals of the patient, as shown in FIG. 10B. In response to the selection of the graphical item of a vital of the patient, the patient monitoring platform 101 displays additional graphical items that allow the care provider to enter information related to the tasks of the vitals. For example, if the care provider selects the graphical item for heart rate (displayed as HR in FIG. 10B), then the patient monitoring platform 101 displays graphical item 1002, as shown in FIG. 10C.

Turning now to FIG. 10C, the graphical item 1002a is associated with creating a task for the patient to monitor his/her vitals. The care provider using the graphical items 1002b adds the vitals that he/she desires the patient to measure, as shown in FIG. 10D. Using graphical item 1002c, the care provider specifies the frequency per day at which the patient should measure his/her vitals. Similarly, the care provider specifies the times at which these tasks should be performed by the patient, which as shown in FIG. 10H is 8:30 AM, 2:30 PM, and 8:30 PM. Using the graphical item 1002e, the care provider identifies the offset from the specified times at which the patient monitoring platform 101 must remind the user or alert the user with a graphical time.

The patient monitoring platform 101 displays graphical items that are configured to receive inputs related to the various threshold ranges within which should be the patient's measurements. Examples of such graphical items are shown in FIGS. 10E, 10F, 10G. The care provider is also able to provide the upper and lower bound threshold values for a healthy heart rate, blood pressure, respiratory, as shown in FIGS. 10E, 10F, 10G, respectively. The patient monitoring platform 101, displays the inputs received by the graphical items shown in FIGS. 10E, 10F, 10G, in the graphical item 1002a, as shown in FIG. 10H. In response to the selection of the graphical item 1002f, the patient monitoring platform 101 displays additional graphical items configured to receive inputs related to whom the alerts should be sent if one of the vitals is outside a threshold range, such as the heart rate being above the upper bound threshold value or below the lower bound threshold value, as shown in FIGS. 10I, 10J, 10K. As shown in FIG. 10K, the care provider may also specify, via the additional graphical items displayed in response to the selection of the graphical item 1002f, a summarized message that the patient monitoring platform 101 should display when the patient monitoring platform 101 transmits the alert to the specified persons.

The graphical item 1002a includes the graphical items 1002g, 1002h. In response to the selection of the graphical items 1002g, patient monitoring platform 101 closes the graphical item 1002a. The patient monitoring platform 101 saves the task information provided using the graphical items 1002a in a data storage unit operably coupled to the patient monitoring platform 101 and closes the graphical item 1002a after a successfully saving the information. The patient monitoring platform 101 displays certain graphical items to indicate to the user, such as the care provider, that one or more tasks for the care plan have been created by the user, such as the care provider. For example, as shown in FIG. 10L, the graphical item 1001n includes graphical items, different from the graphical items in FIG. 10B, to indicate to the care provider that for vitals with the different graphical item, one or more tasks have been created. The patient monitoring platform 101 displays graphical item 1003 displaying summary of the task and associated alerts in response to the selection of the additional graphical item displayed in FIG. 10L, as shown in FIG. 10M. Similar to the graphical items displayed for entering vitals data, the patient monitoring platform 101 displays graphical items for receiving data related to wellness, exercise, diet, medications, as shown FIGS. 10N, 10O, 10P, 10Q, 10R, 10S. The patient monitoring the platform 101 provides to the care provider graphical items to save the care plan. The patient monitoring platform 101 displays a graphical item to the care provider requesting whether the care provider wishes to transmit the care plan to the patient and in response to receiving an input indicating that the care provider approves the transmission of the care plan to the patient, the patient monitoring platform 101 transmits the care plan to the patient.

As described above, the patient monitoring platform 101 displays alerts to a care provider from patients of the care provider. Examples of such alerts are shown in FIG. 11A. Turning now to FIG. 11A, there is shown an application screen 1100a displaying a GUI 1100b presenting alerts for the patient management functionality of the patient monitoring platform 101. The GUI 1100b includes graphical items 1101a, 1101b, 1101c, 1101d. The graphical items 1101a, 1101b, 1101c display alerts received by the care provider. The patient monitoring platform 101 displays information related to the alerts in the graphical items 1101a, 1101b, 1101c, as shown in FIG. 11A. For each of the graphical items 1101a, 1101b, 1101c, the patient monitoring platform 101 displays graphical items that are configured to receive input to communicate with the patient related to the alert. An example of such graphical items is graphical item 1101d. The graphical item 1101d is configured to receive input to initiate a video call with the patient related to the alert displayed by the graphical item 1101a. In response to the selection of the graphical item 1101d, the patient monitoring platform 101 initiates the video call with the patient and displays the GUI 900b, as shown in FIG. 11B.

As described above, the GUI 900b displays video conversation between two users, which in FIG. 11B are a care provider and the patient. While the care provider is discussing the alert with the patient, the care provider may retrieve the care plan associated with the patient to view the patient's data related to the care plan. The patient monitoring platform 101 retrieves and displays the patient's data related to the care plan associated with the patient in response to the selection of graphical items (not shown separately in FIG. 11B) configured to receive input from the care provider for retrieving and displaying the patient's data related to the care plan associated with the patient. FIGS. 11C, 11D, 11E, 11F, 11G display patient's data related to vitals, wellness, exercise, diet, and medication, respectively, that a care provider may view to further diagnose the alert. While communicating with and diagnosing the patient, the care provider may further add to the notes related to the patient using the patient monitoring platform 101, as shown in FIG. 11H. Based on the communication with the patient, examination of the patient's data related to the care plan, and/or diagnosis of the alert, the care provider may modify certain parts of the care plan associated with the patient, an example of which is shown in FIG. 11I. In FIG. 11I, the patient monitoring platform 101 displays modifications a care provider is making to the medication for the patient by updating the medications portion of the care plan.

In some embodiments, the patient monitoring platform 101 displays a navigational graphical item (not shown) associated with the functionality of retrieving data associated with all of the tasks and alerts of a care plan, and data related to a patient's progress through the care plan. In response to the selection of the navigational graphical item, the patient monitoring platform 101 displays a GUI (not shown) configured to present all of the tasks and alerts associated with a care plan, including a patient's progress through the tasks and patient's management of the alerts, on a single application screen. The GUI includes one or more graphical items for each of the tasks of the care plan and in response to the selection of a graphical item, the patient monitoring platform 101 displays additional graphical items that present data related to the task and data related to the patient's performance of the task.

The patient monitoring platform 101 may display the data related to a patient's performance of the task in multiple ways including, but not limited to, graphs, tabular form, etc. In some embodiments, the patient monitoring platform 101 presents each display of data related to patient's performance of a task in graphical items configured to receive inputs to change parameters associated with the data, such as time period, etc., of the data, and the patient monitoring platform 101, in response to receiving the inputs, updates the graphical item with data associated with the new or updated parameter. By displaying data related to all of the tasks, alerts, and a patient's progress through a care plan on a single GUI, the patient monitoring platform 101 provides a clear and uncluttered view of data associated with the care plan, which assists a care provider to easily determine the effectiveness of the care plan, including how well the patient managed to adhere to the care plan.

Typically, an alert may be sent to the care provider based on patient's data related to vitals, wellness, exercise, diet, medication, and follow up encounters. However, such alerts do not provide the care provider with actionable information due to their simplistic nature resulting in patient's overall health being affected. Thus, the patient monitoring platform 101 is further configured to improve patient health through remote monitoring and response thereby improving care provider's situational awareness of the patient's health status e.g., the patient's health along a homeostasis-decompensation continuum, and vice versa. Data sources, such as, for example, (1) the generated care plan, (2) adherence of the care plan, (3) baseline health (objective and subjective), and (4) medical history are incorporated in the patient monitoring platform 101 from which a clinical decision support generates care provider suggestions. The generated care plan, as described above, includes tasks different for each patient, and each task (e.g., medication, vitals, diet, fitness, wellness and follow-up related) generally includes a duration from a current time and baseline health to an end time and updated health.

Baseline health is a combination of objective physiological health measures and subjective health measures at the time of first encounter coupled with the patient's medical history. Objective health includes, for example, heart rate, blood pressure, weight, blood sugar, and blood oxygen rate. Subjective health includes, for example, breathing quality, swelling, wound healing, sleep quality, and sodium intake, which may be based on a scale from Very Low (1) to Very High (5). These lists for the objective and subjective health measures are not exhaustive but may include other health measures as needed

During the duration of the task of the generated care plan, the generated care plan receives input by or gathers data from other sources such as, for example, other care providers, electronic medical records, or medical devices to provide a baseline metrics with respect to: (A) patient health (homeostasis-decompensation continuum), (B) patient adherence to plan, and (C) medical treatments (e.g., utilization of off-site medical facilities, such as, for example hospitals or emergency room). The patient monitoring platform 101 enables providers to establish preferred measures and set in the care plan triggers/alerts against those preferred measures. In some embodiments, the preferred measures are a preferred range. If the patient operates outside the preferred range of the care provider the patient monitoring platform 101 sends alerts to inform care providers that the patient is operating outside established preferred ranges.

In some embodiments, to determine (A) the level of patient health, (B) the level of patient adherence, and (C) number of off-site medical treatment, and the provider responses to triggers/alerts as set in a care plan may be determined based on one or more predetermined criteria set by the care provider, or combination thereof.

For example, a baseline patient information is recorded at first encounter with (A) health: objective: heart rate 63 bpm, blood pressure: 140/90, weight: 190, glucose of 250 mg/dl and blood oxygen of 93% and subjective: breathing quality of 3, swelling of 4, wound heal of 2, sleep quality of 2, and diet sodium intake of 4; (B) adherence baseline of 36% of tasks to be completed (reported electronically to care provider of task completions) in the within the past 30, 60, or 90 day period,(e.g. the care plan having 100 vitals, heart rate, blood pressure, glucose, blood oxygen reporting tasks in which only 36 of the reporting task were completed); and (C) medical treatment: five (5) ER visits within the past 12 months with the discharging diagnosis of Heart Failure, Hypertension, COPD and Diabetes. This baseline patient information, including (A) patient health, (B) patient adherence, and (C) medical treatment is gathered from multiple sources (e.g., electronic medical records and devices) and manually and/or automatically inputted, as a result generating a set of tasks for customization and approval by the care provider. The set of tasks is customized and approved by the care provider for the patient to perform in the form of the care plan. In some embodiments, other data may be integrated into the generated care plan, such as, for example the date, time, and geo-location of the patient. The generated care plan will set a goal for (A) patient health, (B) patient adherence, and (C) medical treatment, and in response to these goals the patient monitoring platform 101 (1) reminds the patient of the task, (2) collects and interprets patient responses, (3) generates a set of clinical decision support suggestions and education material, and (4) distributes the set of clinical decision support suggestions and education materials to care providers, a care team of care providers, and/or patients. For example, the patient's heart rate, blood pressure, glucose, and breathing are consistently out of the care provider's preferred range and/or missed tasks over a consecutive three (3) day period (e.g., patient adherence trigger) culminating in reduced activity levels (e.g., patient adherence trigger or patient health trigger) showing a trend associated with a patients location on those given days the provider will be informed of the circumstances contributing to alert and be presented with clinical decision and lifestyle decision support suggestions.

The predetermined criteria to trigger an alert may include, for example, a total or a consecutive number of missed tasks, which support clinical decisions and approach in addressing the care provider's assessment and plan for the patient. The patient monitoring platform 101 is configured to set a number of task that is allowed to be missed before an alert is sent to the care provider. For example, if the task is to take medication twice a day for thirty days, the patient monitoring platform 101 creates a total of 60 time and date specific medication-related tasks as a part of the care plan. The care provider may set a total number of missed medication-related tasks, e.g., five missed medication-related tasks in that 30 day period, which triggers an alert. Thus, at any point during the 30 days, if medication-related tasks are missed five times, an alert is sent to the care provider. In addition to monitoring completed and incomplete total tasks and resulting measurements, the patient monitoring platform 101 further correlates with additional patient health data and medical treatment data to determine if there is a trend towards patient homeostasis or decompensation.

In embodiments, the care provider may be less concerned about a total number of missed medications than consecutive number of missed medication task. In this case, the predetermined criteria may include a consecutive number of missed tasks, which are allowed to be missed in a row or consecutively before an alert is sent to the care provider. For example, as stated above if the task is to take medication, the care provider sets a total number of consecutively missed medication-related tasks, e.g., five in a row or consecutively. Thus, at any point during the 30 days, when medication-related tasks are consecutively missed five times, an alert is sent to the care provider. In addition to monitoring completed and incomplete total tasks and resulting measurements, the patient monitoring platform 101 further correlates with additional patient health data and medical treatment data to determine if there is a trend towards patient homeostasis or decompensation.

In embodiments, the predetermined criteria to trigger an alert, may further include when the measurement of the task of the generated care plan is outside the preferred objective and/or subjective ranges of the care plan as set by the care provider in the care plan. In some embodiments, the predetermined criteria to trigger an alert may further include any medical treatment data. In the instances of the predetermined criteria to trigger an alert based on the number of missed task, measurement of the task being outside the preferred range as set by the care provider in the care plan, any medical treatment, or any combination thereof, an alert is sent to the care provider. In some embodiments, in response to the alert sent to the care provider, the patient needs to perform additional follow up responsibilities, such as, immediately booking an appointment, contacting the care provider, and/or answering a questionnaire specific to the patient's condition. The patient monitoring platform 101 utilizes the data from the encounter and/or questionnaire to refine situational awareness which support clinical decisions and lifestyle coaching in addressing the care provider's remote treatment of the patient. Additionally based on the data from the encounter and/or questionnaire, the patient monitoring platform 101 produces a (1) self-generated care plan(s) or (2) tasks the patient to book an appointment with a care team member or contact the care provider immediately. The patient monitoring platform 101, captures the care provider's care plan modifications from either (1) or (2) and monitors the patient results. Depending on the monitored patient results and outcomes, the results and outcome is utilized by the patient monitoring platform 101 to contribute to the clinical and lifestyle decision support refinement and adjusted to create evidence based decision support.

The patient monitoring platform 101 may be further configured to generate and provide analytic reports based on (A) patient health outcomes, (B) percentage of total and individual care plan task compliance/adherence, and (C) medical treatment outcomes. The generated reports may be used by the care providers for both population health management and personalized/individual care management. The patient monitoring platform 101 generates and provides reports of trend analysis predictive of apparent or non-apparent clinical decompensation or clinical stability, patterns of non-adherence to medical treatment, patterns of physiologic deterioration or homeostasis. Patient monitoring platform 101 generates and provides reports of correlated variables set by the provider and extracted from data sources in (A), (B) and/or (C). Correlated variable reports show the extent to which the selected data sources in (A), (B) and/or (C) fluctuate in relation to one another and are customizable by the care provider. For example, a care provider may wish to generate a report based on a patient's population of a certain age, diagnosis, location and socio economic status and their adherence to care plan tasks set against their health status (decompensating or homeostasis) and their health status as it relates to medical treatments (e.g. ER visits). Based on the results of this report the provider can then customize patient's care based on that set population.

The patient monitoring platform 101 may additionally be configured to enrich interactions between the patient and the care provider by generating pre-call questions for the patient to answer prior to the upcoming appointment and the patient to provide a multimedia response to the care provider's appointment in the form of sharing images in the appointment form. At the time of the appointment, the patient monitoring platform 101 enables contact of the patient, rescheduling, creating notes, and canceling appointments. All appointments are automatically transferred to all encounter notes.

The patient monitoring platform 101 may additionally be configured to enhance team based care coordination by creating care teams with delegated roles and responsibilities to whom targeted alerts are delivered to the responsible party. The patient monitoring platform 101 automatically generates and provides clinical decision support and related educational materials to assist the care provider in their care plan alert response, as discussed above. Care team coordination enables note creation and sharing, tracking time of all care team interactions, and interoperability with the electronic medical records.

The patient monitoring platform 101 is further configured to provide inter-professional care coordination between care providers. During inter-professional care coordination between care providers, the referring care provider initiates a reimbursement consult with a consulting care provider. In initiating the reimbursement consult, the referring care provider locates a consulting care provider within a network of the patient's providers where the referring care provider can establish communication via the communication systems 110. The referring care provider shares the consulting care provider with the referenced patient's electronic medical records. Any communication between the referring care provider and the consulting care provider is auto-tracked to provide accurate and detailed accounting of their interactions. The consulting care provider, in addition to receiving the electronic medical records of the referenced patient, the consulting care provider may initiate communication with the referring care provider and provide an assessment and plan, before, during, and after the communication with the referring care provider. The assessment and plan generated by the consulting care provider may be made part of the patient's electronic medical records to be shared with any other care provider or necessary parties. The patient monitoring platform 101 captures all interactions time, encounter type across a heterogeneous and homogenous system and generates and provides a report complete with time spent, encounter type, encounter notes, designates treating and consulting care provider and referred patient.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an example,” “in examples,” “in some examples,” “in an embodiment,” “in embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

The systems described herein may also utilize one or more controllers to receive various information and transform the received information to generate an output. The controller may include any type of computing device, computational circuit, or any type of processor or processing circuit described herein. The controller may include multiple processors and/or multicore central processing units (CPUs) and may include any type of processor described herein. The controller may also include a memory to store data and/or instructions that, when executed by the one or more processors, causes the one or more processors to perform one or more methods and/or algorithms.

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

Any of the herein described methods, programs, algorithms, or codes may be contained on one or more machine-readable media or memory described herein. Code or instructions contained thereon can be represented by carrier wave signals, infrared signals, digital signals, and by other like signals.

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A method of presenting information related to a patient record to a user, the method comprising:
displaying a first graphical user interface on a display, the first graphical user interface associated with a first functionality, wherein the first graphical user interface is of a first size;
receiving an input for the selection of a second functionality associated with a second graphical user interface, wherein the second graphical user interface is different from the first graphical user interface;
in response to receiving the input, resizing the first graphical user interface to a second size; and
displaying the second graphical user interface on the display with the resized first graphical user interface, wherein the second graphical user interface is of the second size.
